(19) United States
(12) Patent Application Publication
(10) Pub. No.: US 2021/0215714 A1
(43) Pub. Date: Jul. 15, 2021

(54) COMPLEMENT ALTERNATIVE PATHWAY-ASSOCIATED NEPHROPATHY BIOMARKERS

(71) Applicant: Achillion Pharmaceuticals, Inc., Boston, MA (US)

(72) Inventors: Mingjun HUANG, Potomac, MD (US); Steven D. PODOS, New Haven, CT (US); Wengang YANG, Guilford, CT (US); Yongsen ZHAO, New Haven, CT (US)

(21) Appl. No.: 17/058,547

(22) PCT Filed: May 28, 2019
(86) PCT No.: PCT/US2019/034210

§ 371 Date: Nov. 24, 2020

Related U.S. Application Data
(60) Provisional application No. 62/676,858, filed on May 25, 2018.
(60) Provisional application No. 62/750,048, filed on Oct. 24, 2018.

(51) Int. Cl. G01N 33/68; A61K 31/506; A61K 31/517

(52) U.S. Cl. CPC G01N 33/6893; A61K 31/506; G01N 2800/52; G01N 2333/4716; G01N 2800/347; A61K 31/517

(57) ABSTRACT

Provided herein are methods for using determinative urinary biomarkers for identifying a human subject suffering from an alternative pathway (AP)-associated nephropathy. Also provided herein is a method for using determinative urinary biomarkers to determine whether a human subject suffering from a complement mediated nephropathy is likely to respond to an inhibitor of the alternative complement pathway (“AP”) in treating the complement mediated nephropathy. Further provided herein is a method for using urinary biomarkers to assess the therapeutic response of a human subject suffering from an AP-associated nephropathy receiving inhibitors of the complement alternative pathway (AP inhibitors).

### STATEMENT OF RELATED APPLICATIONS

This application claims the benefit of U.S. Provisional Application No. 62/676,858, filed on May 25, 2018, and U.S. Provisional Application No. 62/750,048, filed Oct. 24, 2018. The entirety of each of these applications is incorporated herein by reference for all purposes.

### FIELD OF THE INVENTION

This invention describes non-invasive urinary biomarkers for identifying human subjects having a complement alternative pathway (AP)-associated nephropathy, for example a component 3 glomerulopathy (C3G) disorder, for example C3 glomerulonephritis (C3GN) or dense deposit disease (DDD), or a membranoproliferative glomerulonephritis (MPGN) disorder, for example immune-complex membranoproliferative glomerulonephritis (IC-MPGN).

### BACKGROUND OF THE INVENTION

An immune disorder occurs when the immune system is not performing in a normal manner. A wide variety of medical disorders are caused by detrimental immune or inflammatory responses, or the inability of a cell to respond to a normal immune or inflammatory process.

The complement system is a part of the innate immune system which does not adapt to changes over the course of the host's life, but is recruited and used by the adaptive immune system. For example, it assists, or complements, the ability of antibodies and phagocytic cells to clear pathogens. This sophisticated regulatory pathway allows rapid reaction to pathogenic organisms while protecting host cells from destruction. Over thirty proteins and protein fragments make up the complement system. These proteins act through opsonization (enhancing phagocytosis of antigens), chemotaxis (attracting macrophages and neutrophils), cell lysis (rupturing membranes of foreign cells) and agglutination (clustering and binding of pathogens together).

The dysfunction of, or excessive activation of, complement has been linked to certain autoimmune, inflammatory, and neurodegenerative diseases, as well as ischemia-reperfusion injury and cancer. Importantly, kidneys are particularly susceptible to excessive complement activation, with many kidney disorders particularly associated with activation of the alternative pathway of the complement system. One exemplary group of such disorders are the complement component 3 glomerulopathies (C3G). C3G is a recently defined entity comprising of dense deposit disease (DDD) and C3 glomerulonephritis (C3GN) which encompasses a population of chronic kidney diseases wherein elevated activity of the alternative complement pathway and terminal complement pathway results in glomerular deposits made solely of complement C3 and no immunoglobulin (Ig). Other exemplary disorders of the kidneys that are associated with complement alternative pathway activation include membranoproliferative glomerulonephritis (MPGN) such as immune complex MPGN (IC-MPGN), atypical or typical hemolytic uremic syndrome (HUS), lupus nephritis, IgA nephropathy, anti-neutrophilic cytoplasmic autoantibody (ANCA) glomerulonephritis, scleroderma renal crisis, post-infectious glomerulonephritis, renal failure resulting from pre-eclampsia or eclampsia, and complications of kidney transplantation such as delayed graft function or risk of thrombus formation.

The key step in the complement cascade is the cleavage of C3 to C3a and C3b leading to the formation of a C3 convertase via the classic, lectin, or alternative complement pathways (CP, LP, or AP, respectively) (see review by Fervenza et al., “Circulating Complement Levels and C3 Glomerulopathy,” Clin J Am Soc Nephrol. 9(11): 1829-1831, 2014). Whereas antigen-antibody complexes and carbohydrate moieties found primarily on the surface of microbial pathogens are needed to activate the CP or LP, respectively, the AP is capable of autoactivation by a mechanism called “tick-over” of C3. This mechanism occurs spontaneously at a low rate, generating a conformationally changed C3 capable of binding complement factor B (fB), resulting in the cleavage of fB by factor D, and generating Ba and Bb. This leads to the formation of the AP C3 convertase (C3bBb).

In light of their early and central role in activation of the alternative pathway of the complement cascade, complement factor D (fD), complement factor B (fB), complement factor H (fH), component 3 (C3), component 3b (C3b), and the C3 convertase are all attractive targets for inhibition or regulation of the complement cascade, and have been contemplated for the treatment of complement alternative pathway (AP)-associated nephropathies.

Unfortunately, definitive diagnosis of AP-associated nephropathies requires invasive and painful kidney biopsies. Nonetheless, a number of potential blood or plasma biomarkers for use in identifying these disorders have been examined (see Zhang et al., Defining the Complement Biomarker Profile of C3 Glomerulopathy, Clin J Am Soc Nephrol. 2014 Nov. 7; 9(11): 1876-1882; Caliskan et al., Novel Biomarkers in Glomerular Disease, Adv Chronic Kidney Dis. 2014 March; 21(2): 205-216). For example, alternative pathway functional assay and assays for circulating complement breakdown products such as C3a, C5a, and soluble form of membrane-attack complex (sMAC or sC5b-9) have been examined as serum biomarkers for C3G and/or MPGN disease and treatment monitoring (Fakhouri et al., C3 glomerulopathy: a new classification, Nat Rev Nephrol. 2010 August; 6(8):494-9; Sethi et al., Membranoproliferative glomerulonephritis—a new look at an old entity, N Engl J Med. 2012 Mar. 22; 366(12):1119-31). Blood based levels of circulating complement components for AP-associated nephropathies, however, may not always be indicative of disease state or responsiveness to treatment, making the monitoring of treatment efficacy and the indication that therapeutic dosage may need to be adjusted both difficult and uncertain. In addition, definitive urinary biomarkers in patients with AP-associated nephropathies have not been identified (Caliskan et al., Novel Biomarkers in Glomerular Disease, Adv Chronic Kidney Dis. 2014 March; 21(2): 205-216).

Accordingly, it would be advantageous to provide definitive biomarkers that are indicative of the level of responsiveness to treatment with alternative complement factor inhibitors in a human subject suffering from an alternative pathway-associated nephropathy.

### SUMMARY OF THE INVENTION

Provided herein are methods for using determinative urinary biomarkers for identifying a human subject suffering from an alternative pathway (AP)-associated nephropathy. Also provided herein is a method for using determinative urinary biomarkers to determine whether a human subject suffering from a complement mediated nephropathy is likely to respond to an inhibitor of the alternative complement pathway (“AP”) in treating the complement mediated nephropathy. Further provided herein is a method for using urinary biomarkers to assess or monitor the therapeutic response of a human subject suffering from an AP-associated nephropathy receiving inhibitors of the complement alternative pathway (AP inhibitors). It has been discovered that monitoring certain complement components and/or breakdown products in the urine of subjects suffering from an AP-associated nephropathy provides a superior method, as compared to for example serum, for determining a diagnosis of an AP-associated nephropathy, determining the stage and severity of the disease, determining patient appropriateness for and responsiveness to treatment with an alternative pathway inhibitor or a combination of AP inhibitors (see for example, Examples 3 and 4 and FIGS. 4A-4F and 5A-5F). In particular, it has been discovered that the urine levels of the complement breakdown products Ba (Ba), and/or C3c (C3c), and/or the complement component soluble C5b-9 (sC5b-9), or a combination of two or more thereof, provide a predictive assessment of both disease state and treatment response. (See for example, Examples 4, 5, and 6 and FIGS. 5A-5F, 6A-6D, and 7A-7C). Accordingly, by measuring these complement components and/or breakdown products in the urine from a subject suspected of having an AP-associated nephropathy, the probable effectiveness of AP inhibitor therapy for the subject can be assessed, allowing for the targeted selection of subjects and the institution of targeted treatment regimens resulting in improved responsiveness to AP inhibition. Likewise, by measuring these complement components and/or breakdown products in the urine in a subject currently receiving an AP inhibitor, the effectiveness of the administered therapeutic regimen can be assessed and, if necessary, adjusted. Additionally, by monitoring urine levels of Ba, sC5b-9, and/or C3c, subjects having chronic kidney disorders can be assessed for their risk of developing end stage renal disease (ESRD) and treated accordingly. In some embodiments, the AP-mediated disorder is a complement component 3 glomerulopathy (C3G). In some embodiments, the AP-mediated disorder is dense deposit disease (DDD) or C3 glomerulonephritis (C3GN). In some embodiments, the AP-mediated disorder is membranoproliferative glomerulonephritis (MPGN) such as immune complex MPGN (IC-MPGN).

By assessing the levels of one or more of the complement components and/or breakdown products Ba, sC5b-9, and/or C3c in the urine of a human subject suspected of having an AP-associated nephropathy, the proper therapeutic protocol for the subject can be prescribed. For example, if the urine levels of Ba, C3c, and/or sC5b-9 are indicative that the subject is suffering from an AP-associated nephropathy, the subject can be administered an appropriate medication targeting AP, for example an AP inhibitor. AP-associated nephropathies that may be discerned through the assessment of the described urinary biomarkers includes, but are not limited to, C3G; C3GN; dense deposit disease (DDD); IC-MPGN; atypical or typical hemolytic uremic syndrome (HUS); lupus nephritis resulting from systemic lupus erythematous (SLE); IgA nephropathy; anti-neutrophilic cytoplasmic autoantibody (ANCA) glomerulonephritis; scleroderma renal crisis; post-infectious glomerulonephritis; glomerulonephritis and vasculitis resulting from Henoch-Schonlein purpura (HSP); anti-glomerular basement membrane (GBM) or Goodpasture's disease; light chain deposition disease; contrast-induced nephropathy (CIN); membranous glomerulonephritis; cryoglobulinemia; pre-eclampsia and eclampsia; and minimal change disease. Examples of AP inhibitors that may be administered to a subject determined to have an AP-mediated disorder through the assessment of the described urinary biomarkers include, but are not limited to, C3 inhibitors, C3b inhibitors, factor H (fH) inhibitors, factor D (fD) inhibitors, factor B (fB) inhibitors, C3 convertase inhibitors, or Bb inhibitors. In particular embodiments, the AP inhibitor administered is a fB inhibitor and/or fD inhibitor. In particular embodiments, the AP inhibitor is selected from Formula I, Formula II, and Compounds 1-25. In some embodiments, the fD inhibitor is Compound 1. In some embodiments, the AP-associated nephropathy is a C3 glomerulopathy (C3G), for example dense deposit disorder (DDD) or C3 glomerulonephritis (C3GN). In some embodiments, the AP-associated nephropathy is immune-complex membranoproliferative glomerulonephritis (IC-MPGN).

Accordingly, in a first aspect of the present invention, methods are provided for the targeted selection and treatment of a human subject suffering from a suspected AP-associated nephropathy that include:
i. analyzing the biomarker levels in the urine of the subject, selected from Ba, sC5b-9, and C3c, alone or in combination;
ii. comparing the subject's biomarker urine levels to a range of the same biomarker urine levels derived from individuals that do not have an AP-associated nephropathy (“normal range”); and
iii. if the subject's urine biomarker levels are greater than the normal range, administering to the subject an AP inhibitor.

In some embodiments, the urine biomarker analyzed is Ba. In some embodiments, the urine biomarker analyzed is sC5b-9. In some embodiments, the urine biomarker analyzed is C3c. In some embodiments, the biomarkers analyzed are at least two of Ba, C3c, and sC5b-9. In some embodiments, the urine biomarkers analyzed are Ba and sC5b-9. In some embodiments, the urine biomarkers analyzed are Ba and C3c. In some embodiments, the urine biomarkers analyzed are sC5b-9 and C3c. In some embodiments, the urine biomarkers analyzed are Ba, sC5b-9, and C3c. In some embodiments, the subject is administered an AP inhibitor if the subject's urine biomarker levels are greater than the upper limit of the normal (ULN) range. In some embodiments, the subject's urine biomarker levels are at least 2×, 3×, 4×, 5×, 6×, 7×, 8×, 9×, 10×, or >10× higher than the upper limit of normal. In some embodiments, the subject's urine biomarker levels are at least 100×, 200×, 300×, 400×, >500× higher than the upper limit of normal. In some embodiments, one or more of the measured biomarkers are greater than the normal range. In some embodiments, two or more the measured biomarkers are greater than the normal range. In some embodiments, each of the measured biomarkers are greater than the normal range. Normal ranges of the urinary biomarkers are readily determinable as described below. Exemplary normal ranges of the biomarkers using the described assays are provided further below. In some embodiments, the AP inhibitor administered is selected from Formula I, Formula II, and Compounds 1-25.

In another aspect of the present invention, methods are provided for the targeted selection and treatment of a human subject suffering from a suspected AP-associated nephropathy that include:
i. analyzing the level of biomarkers in the urine of the subject, selected from Ba, sC5b-9, and C3c, alone or in combination;
ii. comparing the subject's biomarker urine levels to a range of biomarker urine levels derived from individuals that have an AP-associated nephropathy (“abnormal range”); and
iii. if the subject's biomarker urine levels fall within the abnormal range, administering to the subject an AP inhibitor.

In some embodiments, the urine biomarker analyzed is Ba. In some embodiments, the urine biomarker analyzed is sC5b-9. In some embodiments, the urine biomarker analyzed is C3c. In some embodiments, the biomarkers analyzed are at least two of Ba, C3c, and sC5b-9. In some embodiments, the urine biomarkers analyzed are Ba and sC5b-9. In some embodiments, the urine biomarkers analyzed are Ba and C3c. In some embodiments, the urine biomarkers analyzed are sC5b-9 and C3c. In some embodiments, the urine biomarkers analyzed are Ba, sC5b-9, and C3c. In some embodiments, one or more of the measured biomarkers are within the abnormal range. In some embodiments, two or more the measured biomarkers are within the abnormal range. In some embodiments, each of the measured biomarkers are within the abnormal range. In some embodiments, the AP inhibitor administered is selected from Formula I, Formula II, and Compounds 1-25.

As provided herein, subject's with elevated urine biomarker levels may be suitable or susceptible to treatment with an AP inhibitor described herein, and readily identified with a simple urine screen and administered appropriate treatment quickly. In some embodiments, the AP inhibitor is a fD inhibitor. In some embodiments, the AP inhibitor is a fB inhibitor. In some embodiments, a fD inhibitor is combined with another AP inhibitor. In some embodiments, a fD inhibitor is combined with a fB inhibitor. In some embodiments, the fD inhibitor is the AP inhibitor selected from Formula I, Formula II, and Compounds 1-25. In some embodiments, the fD inhibitor is Compound 1. In some embodiments, the AP-associated nephropathy is a C3 glomerulopathy (C3G), for example dense deposit disorder (DDD) or C3 glomerulonephritis (C3GN). In some embodiments, the AP-associated nephropathy is immune-complex membranoproliferative glomerulonephritis (IC-MPGN).

In a further aspect of the present invention, provided herein is a method of monitoring the efficacy of an AP inhibitor treatment regimen in a human subject having an AP-associated nephropathy and receiving an AP inhibitor that includes:
i. analyzing biomarker levels in a first urine sample from the subject, selected from Ba, sC5b-9, and C3c, alone or in combination;
ii. analyzing the same biomarker levels in a subsequent urine sample from the subject, wherein the first urine sample is collected prior to the collection of the subsequent urine sample;
iii. comparing biomarker levels in the first urine sample to biomarker levels in a subsequent urine sample from the subject; and
iv. if the biomarker levels in the subsequent urine sample are equal to or greater than the biomarker levels in the first urine sample, then increasing the dose of the AP inhibitor regimen administered to the subject.

In some embodiments, the first urine sample is collected at baseline, that is, prior to the administration of an AP inhibitor therapeutic regimen. In some embodiments, the first urine sample is collected after the administration of an AP inhibitor therapeutic regimen. In some embodiments, the urine biomarker analyzed is Ba. In some embodiments, the urine biomarker analyzed is sC5b-9. In some embodiments, the urine biomarker analyzed is C3c. In some embodiments, the biomarkers analyzed are at least two of Ba, C3c, and sC5b-9. In some embodiments, the urine biomarkers analyzed are Ba and sC5b-9. In some embodiments, the urine biomarkers analyzed are Ba and C3c. In some embodiments, the urine biomarkers analyzed are sC5b-9 and C3c. In some embodiments, the urine biomarkers analyzed are Ba, sC5b-9, and C3c. In some embodiments, one or more of the measured biomarkers in the subsequent sample are equal to or greater than the levels of the measured biomarkers in the first sample. In some embodiments, two or more of the measured biomarkers in the subsequent sample are equal to or greater than the levels of the measured biomarkers in the first sample. In some embodiments, each of the measured biomarkers in the subsequent sample are equal to or greater than the levels of the measured biomarkers in the first sample. In some embodiments, the AP inhibitor is a fD inhibitor. In some embodiments, the AP inhibitor is a fB inhibitor. In some embodiments, a fD inhibitor is combined with another AP inhibitor. In some embodiments, a fD inhibitor is combined with a fB inhibitor. In some embodiments, the fD inhibitor is selected from Formula I, Formula II, and Compounds 1-25. In some embodiments, the fD inhibitor is Compound 1. In some embodiments, the AP-associated nephropathy is a C3 glomerulopathy (C3G), for example dense deposit disorder (DDD) or C3 glomerulonephritis (C3GN). In some embodiments, the AP-associated nephropathy is immune-complex membranoproliferative glomerulonephritis (IC-MPGN).

In another aspect of the present invention, provided herein is a method of monitoring the efficacy of an AP inhibitor treatment regimen in a human subject having an AP-associated nephropathy and receiving an AP inhibitor that includes:
i. analyzing biomarker levels in a first urine sample from the subject, selected from Ba, sC5b-9, and C3c, alone or in combination;
ii. analyzing the same biomarker levels in a subsequent urine sample from the subject, wherein the first urine sample is collected prior to the collection of subsequent urine samples;
iii. comparing biomarker levels in the first urine sample to biomarker levels in subsequent urine samples; and
iv. if the biomarker levels in the subsequent urine samples have insufficiently decreased compared to the biomarker levels in the first urine sample, then increasing the dose of the AP inhibitor administered to the subject.

In some embodiments, the first urine sample is collected at baseline, that is, prior to the administration of an AP inhibitor therapeutic regimen. In some embodiments, the first urine sample is collected after the administration of an AP inhibitor therapeutic regimen. In some embodiments, the urine biomarker analyzed is Ba. In some embodiments, the urine biomarker analyzed is sC5b-9. In some embodiments, the urine biomarker analyzed is C3c. In some embodiments, the biomarkers analyzed are at least two of Ba, C3c, and sC5b-9. In some embodiments, the urine biomarkers analyzed are Ba and sC5b-9. In some embodiments, the urine biomarkers analyzed are Ba and C3c. In some embodiments, the urine biomarkers analyzed are sC5b-9 and C3c. In some embodiments, the urine biomarkers analyzed are Ba, sC5b-9, and C3c. In some embodiments the urine biomarkers analyzed have decreased by less than about 10%, 20%, 30%, 40%, 50%, or 60%. In some embodiments, one or more of the measured biomarkers in the subsequent sample have insufficiently decreased compared to the levels of the measured biomarkers in the first sample. In some embodiments, two or more the measured biomarkers in the subsequent sample have insufficiently decreased compared to the levels of the measured biomarkers in the first sample. In some embodiments, each of the measured biomarkers in the subsequent sample have insufficiently decreased compared to the levels of the measured biomarkers in the first sample. In some embodiments, the insufficient decrease is a biomarker level above the normal range. In some embodiments, the AP inhibitor is a fD inhibitor. In some embodiments, the AP inhibitor is a fB inhibitor. In some embodiments, a fD inhibitor is combined with another AP inhibitor. In some embodiments, a fD inhibitor is combined with a fB inhibitor. In some embodiments, the AP inhibitor is selected from Formula I, Formula II, and Compounds 1-25. In some embodiments, the fD inhibitor is Compound 1. In some embodiments, the AP-associated nephropathy is a C3 glomerulopathy (C3G), for example dense deposit disorder (DDD) or C3 glomerulonephritis (C3GN). In some embodiments, the AP-associated nephropathy is immune-complex membranoproliferative glomerulonephritis (IC-MPGN).

Importantly, as provided herein, the subject's urine biomarker levels can be monitored during an AP inhibitor treatment regimen and compared to previous or baseline measurements to determine if the subject is responding to therapy. If the subject is sufficiently responding to AP inhibitor therapy, the subject's biomarker levels will show a significant decrease compared to baseline or previous levels, for example, 25%, 35%, 50%, or greater, and the subject can be maintained on the current dosing regimen. If the subject is not responding or responding insufficiently, however, the subject can be administered an increased dosage of an AP inhibitor, or extended on the current dosage for an increased period of time. For example, if the subject is receiving Compound 1 at a dosage of 100 mg three times daily, and the subject's biomarker levels do not show a significant or sufficient decrease compared to baseline or previous levels, the subject's dosage of Compound 1 can be increased to, for example, 150 mg or 200 mg three times daily.

In another aspect of the present invention, methods are provided for diagnosing a human subject suffering from a suspected AP-associated nephropathy that include:
i. analyzing the biomarker levels in the urine of the subject, selected from Ba, sC5b-9, and C3c, alone or in combination;
ii. comparing the subject's biomarker urine levels to a range of the same biomarker urine levels derived from individuals that do not have an AP-associated nephropathy (“normal range”); and
iii. diagnosing an AP-associated nephropathy if the subject's urine biomarker levels are greater than the normal range.

In some embodiments, the urine biomarker analyzed is Ba. In some embodiments, the urine biomarker analyzed is sC5b-9. In some embodiments, the urine biomarker analyzed is C3c. In some embodiments, the biomarkers analyzed are at least two of Ba, C3c, and sC5b-9. In some embodiments, the urine biomarkers analyzed are Ba and sC5b-9. In some embodiments, the urine biomarkers analyzed are Ba and C3c. In some embodiments, the urine biomarkers analyzed are sC5b-9 and C3c. In some embodiments, the urine biomarkers analyzed are Ba, sC5b-9, and C3c. In some embodiments, the subject's urine biomarker levels are at least 2×, 3×, 4×, 5×, 6×, 7×, 8×, 9×, 10×, or >10× higher than the upper limit of normal. In some embodiments, the subject's urine biomarker levels are at least 100×, 200×, 300×, 400×, >500× higher than the upper limit of normal. In some embodiments, one or more of the measured biomarkers are greater than the normal range. In some embodiments, two or more the measured biomarkers are greater than the normal range. In some embodiments, each of the measured biomarkers are greater than the normal range. In some embodiments, the AP-associated nephropathy is a C3 glomerulopathy (C3G), for example dense deposit disorder (DDD) or C3 glomerulonephritis (C3GN). In some embodiments, the AP-associated nephropathy is immune-complex membranoproliferative glomerulonephritis (IC-MPGN).

In another aspect of the present invention, methods are provided for the diagnosis of a human subject suffering from a suspected AP-associated nephropathy that include:
i. analyzing the biomarker levels in the urine of the subject, selected from Ba, sC5b-9, and C3c, alone or in combination;
ii. comparing the subject's biomarker urine levels to a range of the same biomarker urine levels derived from individuals that do have an AP-associated nephropathy (“abnormal range”); and
iii. diagnosing an AP-associated nephropathy if the subject's urine biomarker levels are within the abnormal range.

In some embodiments, the urine biomarker analyzed is Ba. In some embodiments, the urine biomarker analyzed is sC5b-9. In some embodiments, the urine biomarker analyzed is C3c. In some embodiments, the biomarkers analyzed are at least two of Ba, C3c, and sC5b-9. In some embodiments, the urine biomarkers analyzed are Ba and sC5b-9. In some embodiments, the urine biomarkers analyzed are Ba and C3c. In some embodiments, the urine biomarkers analyzed are sC5b-9 and C3c. In some embodiments, the urine biomarkers analyzed are Ba, sC5b-9, and C3c. In some embodiments, one or more of the measured biomarkers are within the abnormal range. In some embodiments, two or more the measured biomarkers are within the abnormal range. In some embodiments, each of the measured biomarkers are within the abnormal range. In some embodiments, the AP-associated nephropathy is a C3 glomerulopathy (C3G), for example dense deposit disorder (DDD) or C3 glomerulonephritis (C3GN). In some embodiments, the AP-associated nephropathy is immune-complex membranoproliferative glomerulonephritis (IC-MPGN).

In another aspect of the present invention, methods are provided for determining the risk of a subject having an AP-associated nephropathy of progressing to end stage renal disease that include:
i. analyzing the human subject's urine biomarker levels, selected from Ba, sC5b-9, and C3c, alone or in combination;
ii. comparing the subject's biomarker urine levels to a range of the same biomarker urine levels derived from individuals that do not have an AP-associated nephropathy (“normal range”); and
iii. if the subject's biomarker urine levels are greater than the normal range, identifying the subject as at risk for developing end stage renal disease.

In some embodiments, the urine biomarker analyzed is Ba. In some embodiments, the urine biomarker analyzed is sC5b-9. In some embodiments, the urine biomarker analyzed is C3c. In some embodiments, the biomarkers analyzed are at least two of Ba, C3c, and sC5b-9. In some embodiments, the urine biomarkers analyzed are Ba and sC5b-9. In some embodiments, the urine biomarkers analyzed are Ba and C3c. In some embodiments, the urine biomarkers analyzed are sC5b-9 and C3c. In some embodiments, the urine biomarkers analyzed are Ba, sC5b-9, and C3c. In some embodiments, the subject's urine biomarker levels are at least 2×, 3×, 4×, 5×, 6×, 7×, 8×, 9×, 10×, or >10× higher than the upper limit of normal. In some embodiments, the subject's urine biomarker levels are at least 100×, 200×, 300×, 400×, >500× higher than the upper limit of normal. In some embodiments, one or more of the measured biomarkers are greater than the normal range. In some embodiments, two or more the measured biomarkers are greater than the normal range. In some embodiments, each of the measured biomarkers are greater than the normal range. In some embodiments, if the subject is determined to be at risk of developing end stage renal disease, administering to the subject an AP-inhibitor. In some embodiments, the AP inhibitor is a fD inhibitor. In some embodiments, the AP inhibitor is a fB inhibitor. In some embodiments, a fD inhibitor is combined with another AP inhibitor. In some embodiments, a fD inhibitor is combined with a fB inhibitor. In some embodiments, the fD inhibitor is selected from Formula I, Formula II, and Compounds 1-25. In some embodiments, the fD inhibitor is Compound 1. In some embodiments, the AP-associated nephropathy is a C3 glomerulopathy (C3G), for example dense deposit disorder (DDD) or C3 glomerulonephritis (C3GN). In some embodiments, the AP-associated nephropathy is immune-complex membranoproliferative glomerulonephritis (IC-MPGN).

In one aspect, provided herein is a method of treating a subject with an alternative pathway-associated nephropathy comprising administering to the subject a fD inhibitor; wherein the subject at the time of administration of the fD inhibitor has been, or is currently, receiving a therapeutic regimen comprising the administration of a C5 inhibitor; and wherein the Ba level in the subject's urine is greater than the range of Ba urine levels derived from individuals that do not have an alternative pathway-associated nephropathy. In some embodiments, the subject's urine biomarker levels are at least 2×, 3×, 4×, 5×, 6×, 7×, 8×, 9×, 10×, or >10× higher than the upper limit of normal. In some embodiments, the subject's urine biomarker levels are at least 100×, 200×, 300×, 400×, >500× higher than the upper limit of normal. In particular embodiments, the complement AP inhibitor is selected from Formula I, Formula II, and Compounds 1-25. In some embodiments, the fD inhibitor is Compound 1. The ability to administer a fD inhibitor described herein upon the measurement of abnormal levels of Ba in the urine while receiving a C5 inhibitor provides an effective therapeutic agent switching strategy, extending the effectiveness of the C5 inhibitor and providing additional therapeutic options for subjects with AP-associated nephropathies showing suboptimal response to the C5 inhibitor. In some embodiments, the AP-associated nephropathy is a C3 glomerulopathy (C3G), for example dense deposit disorder (DDD) or C3 glomerulonephritis (C3GN). In some embodiments, the AP-associated nephropathy is immune-complex membranoproliferative glomerulonephritis (IC-MPGN).

As water reabsorption in kidneys affect urinary solute concentrations, urinary biomarker concentrations are frequently reported as a ratio to urinary creatinine (uCr). Accordingly, in any of the above aspects, in embodiments thereof, any of a subject's urine Ba, sC5b-9, or C3c levels can be analyzed, either individually or in various combinations, including analyzing at least two of the three or all three biomarkers. In some embodiments, the subject's urine sC5b-9 level is analyzed. In alternative embodiments, the subject's urine Ba level is analyzed. In other alternative embodiments, the subject's urine C3c level is analyzed. In some embodiments, the biomarkers analyzed or at least two of Ba, C3c, and sC5b-9. Alternatively, the subject's urine sC5b-9 and urine Ba levels are analyzed. Alternatively, the subject's urine sC5b-9 and urine C3c level is analyzed. Alternatively, the subject's urine Ba and urine C3c level is analyzed. In still other alternative embodiments, the subject's urine sC5b-9, urine Ba, and urine C3c levels are analyzed. In certain embodiments, the subject's urine Ba, urine sC5b-9, and/or urine C3c levels can be normalized. In some embodiments, the subject's urine Ba, urine sC5b-9, and/or urine C3c levels are normalized to urine creatinine. In some embodiments, the subject's urine Ba, urine sC5b-9, and/or urine C3c levels are normalized to urine albumin. In some embodiments, the subject's urine Ba, urine sC5b-9, and/or urine C3c levels are normalized to both urine creatinine and urine albumin.

As provided herein, the methods recited herein can be utilized to treat an AP-associated nephropathy, including both C3-associated and C4-associated nephropathies as described further below. In certain embodiments of any of the aspects described above, the AP-associated nephropathy is a C3G or MPGN disorder. In some embodiments, the C3G disorder is C3 glomerulonephritis (C3GN). In some embodiments, the C3G disorder is Dense Deposit Disease (DDD). In some embodiments, the MPGN disorder is IC-MPGN. In some embodiments, the AP-associated nephropathy is chronic kidney disease.

The methods recited herein can be utilized with all types of AP inhibitors, or combinations thereof. In addition, to the extent a subject is currently receiving a complement inhibitor, for example, a C3 or C5 inhibitor, for example the C5 inhibitor eculizumab, the subject's urine Ba levels, C3c levels, and/or sC5b-9 levels, or a combination thereof, can be monitored to determine whether the subject would benefit from the addition of, or conversion to, an AP inhibitor. For example, if the subject's urine Ba biomarker level begins to increase, or fails to decrease, while receiving the C5 or C3 inhibitor, the subject may be further administered an AP inhibitor. In some embodiments, the AP inhibitor is a fD inhibitor. In some embodiments, the AP inhibitor is a fB inhibitor. In particular embodiments, the fD inhibitor is selected from Formula I, Formula II, Compounds 1-25, an fD inhibitor from BioCryst Pharmaceuticals as described below, an fD inhibitor from Novartis as described below, an fD inhibitor from Bristol-Myers Squibb as described below, an fD inhibitor from Japan Tobacco Inc. as described below, FCFD4515S, nafomostat, SOMAmers for fD (SomaLogic), lampalizumab, aptamers to fD (Vitrisa Therapeutics), an fD inhibitor from Ra Pharmaceutical as described below, an fD inhibitor from Alexion Pharmaceuticals as described below, and an fD inhibitor from Achillion Pharmaceuticals as described below. In some embodiments, the fD inhibitor is selected from Formula I, Formula II, and Compounds 1-25. In some embodiments, the fD inhibitor is Compound 1.

In particular embodiments, the fB inhibitor from anti-FB siRNA, TA106, LNP106, LNP023, complin, and Ionis-FB-LRx. In some embodiments, the fB inhibitor is

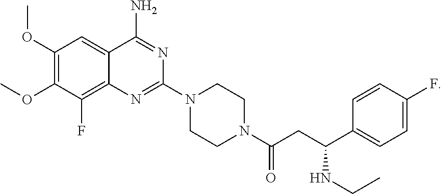 [SMILES: CCN[C@H](CC(=O)N1CCN(c2nc(N)c3cc(OC)c(OC)c(F)c3n2)CC1)c1ccc(F)cc1]

### BRIEF DESCRIPTION OF THE FIGURES

FIG. 1 is a schematic of the generation of the complement pathways. AP activation, directly or in amplification of the classical and lectin pathways, initiates a series of proteolytic events that result in the fD-dependent formation of fluid and membrane-bound C3 convertases, C3(H₂O) Bb and C3bBb. The C3 convertases catalyze C3 cleavage and promote downstream events which lead to C3 fragment deposition, inflammatory activation, and assembly of lytic terminal complement complexes (C5b-9). AP activity is tightly regulated by soluble and membrane regulatory proteins that protect against complement-mediated tissue injury. C3GN is associated with hereditary or acquired AP dysregulation, the latter mediated by stabilizing or inactivating autoantibodies against complement components and regulators. Compound 1 inhibits fD proteolytic activity, prevents fB cleavage at multiple steps of the cascade, prevents AP convertase formation, and thereby effectively blocks AP activity.

FIG. 2 is a schematic of the Phase 2a Proof-of-mechanism study to determine the effect of Compound 1 on C3 levels in patients with C3 glomerulonephritis (C3GN) or Immune complex mediated membranoproliferative glomerulonephritis (IC-MPGN).

FIG. 3A is a plot showing the reduction of albumin-to-creatinine ratio (ACR) with 14-day Compound 1 treatment in Patient A at defined timepoints of the Phase 2a study including screening, treatment, taper period and follow up. The x-axis is ACR measured in ng/mmol and the y-axis is measured in days.

FIG. 3B is a plot showing the reduction of albumin-to-creatinine ratio (ACR) with 14-day Compound 1 treatment in Patient B at defined timepoints of the Phase 2a study including screening, treatment, taper period and follow up. The x-axis is ACR measured in ng/mmol and the y-axis is measured in days.

FIG. 3C is a plot showing the reduction of albumin-to-creatinine ratio (ACR) with 14-day Compound 1 treatment in Patient C at defined timepoints of the Phase 2a study including screening, treatment, taper period and follow up. The x-axis is ACR measured in ng/mmol and the y-axis is measured in days.

FIG. 3D is a plot showing the reduction of albumin-to-creatinine ratio (ACR) with 14-day Compound 1 treatment in Patient D at defined timepoints of the Phase 2a study including screening, treatment, taper period and follow up. It also includes urinary proteins measured on days 14 and 17. The left x-axis is ACR measured in ng/mmol, the right x-axis is 24 hr. urinary proteins measured in g/day and the y-axis is measured in days.

FIG. 4A is a plot of the mean (n=4) ex vivo Ba serum production levels with 14-day Compound 1 treatment at defined timepoints of the Phase 2a study including screening, treatment, taper period and follow up. The x-axis is Ba serum levels measured in μg/ml and the y-axis is measured in days.

FIG. 4B is a plot of the mean (n=4) Bb plasma levels with 14-day Compound 1 treatment at defined timepoints of the Phase 2a study including screening, treatment, taper period and follow up. The x-axis is Bb plasma levels measured in μg/ml and the y-axis is measured in days.

FIG. 4C is a plot of the mean (n=4) % Fragment C3 levels with 14-day Compound 1 treatment at defined timepoints of the Phase 2a study including screening, treatment, taper period and follow up. The x-axis is C3b/iC3b levels measured in percent and the y-axis is measured in days.

FIG. 4D is a plot of the mean (n=4) C3 serum levels with 14-day Compound 1 treatment at defined timepoints of the Phase 2a study including screening, treatment, taper period and follow up. The x-axis is C3 serum levels measured in mg/mL and the y-axis is measured in days.

FIG. 4E is a plot of the mean (n=4) fD serum levels with 14-day Compound 1 treatment at defined timepoints of the Phase 2a study including screening, treatment, taper period and follow up. The x-axis is fD serum levels measured in μg/mL and the y-axis is measured in days.

FIG. 4F is a plot of the mean (n=4) C4 serum levels with 14-day Compound 1 treatment at defined timepoints of the Phase 2a study including screening, treatment, taper period and follow up. The x-axis is C4 serum levels measured in mg/dL and the y-axis is measured in days.

FIG. 5A is a plot of the mean non-normalized (n=6) Ba urinary levels with 14-day Compound 1 treatment at defined timepoints of the Phase 2a study including screening, treatment, taper period and follow up. The x-axis is Ba urinary levels measured in ng/mL and the y-axis is measured in days.

FIG. 5B is a plot of the mean (n=6) Ba urinary levels normalized to creatinine with 14-day Compound 1 treatment at defined timepoints of the Phase 2a study including screening, treatment, taper period and follow up. The x-axis is Ba/Cr urinary levels measured in μg/mmol and the y-axis is measured in days.

FIG. 5C is a plot of the mean (n=6) Ba urinary levels normalized to albumin with 14-day Compound 1 treatment at defined timepoints of the Phase 2a study including screening, treatment, taper period and follow up. The x-axis is Ba/Albumin urinary levels measured in ng/mg and the y-axis is measured in days.

FIG. 5D is a plot of the mean non-normalized (n=6) sC5b-9 urinary levels with 14-day Compound 1 treatment at defined timepoints of the Phase 2a study including screening, treatment, taper period and follow up. The x-axis is sC5b-9 urinary levels measured in ng/mL and the y-axis is measured in days.

FIG. 5E is a plot of the mean (n=6) sC5b-9 urinary levels normalized to creatinine with 14-day Compound 1 treatment at defined timepoints of the Phase 2a study including screening, treatment, taper period and follow up. The x-axis is sC5b-9/Cr urinary levels measured in μg/mmol and the y-axis is measured in days.

FIG. 5F is a plot of the mean (n=6) sC5b-9 urinary levels normalized to albumin with 14-day Compound 1 treatment at defined timepoints of the Phase 2a study including screening, treatment, taper period and follow up. The x-axis is sC5b-9/Albumin urinary levels measured in ng/mg and the y-axis is measured in days.

FIG. 6A is a plot of the highest levels of the non-normalized complement activation products (Ba and sC5b-9) in urine of Patient B with 14-day Compound 1 treatment at defined timepoints of the Phase 2a study including screening, treatment, taper period and follow up. The left x-axis is Ba urinary levels measured in ng/mL, the right x-axis sC5b-9 urinary levels measured in ng/mL and the y-axis is measured in days.

FIG. 6B is a plot of the highest levels of the complement activation products (Ba and sC5b-9) normalized to creatinine in urine of Patient B with 14-day Compound 1 treatment at defined timepoints of the Phase 2a study including screening, treatment, taper period and follow up. The left x-axis is Ba/Cr urinary levels measured in μg/mmol, the right x-axis sC5b-9/Cr urinary levels measured in μg/mmol and the y-axis is measured in days.

FIG. 6C is a plot of the lowest levels of the non-normalized complement activation products (Ba and sC5b-9) in urine of Patient C with 14-day Compound 1 treatment at defined timepoints of the Phase 2a study including screening, treatment, taper period and follow up. The left x-axis is Ba urinary levels measured in ng/mL, the right x-axis sC5b-9 urinary levels measured in ng/mL and the y-axis is measured in days.

FIG. 6D is a plot of the highest levels of the complement activation products (Ba and sC5b-9) normalized to creatinine in urine of Patient C with 14-day Compound 1 treatment at defined timepoints of the Phase 2a study including screening, treatment, taper period and follow up. The left x-axis is Ba/Cr urinary levels measured in μg/mmol, the right x-axis sC5b-9/Cr urinary levels measured in μg/mmol and the y-axis is measured in days.

FIG. 7A is a plot of the mean non-normalized (n=6) C3c urinary levels with 14-day Compound 1 treatment at defined timepoints of the Phase 2a study including screening, treatment, taper period and follow up. The x-axis is C3c urinary levels measured in ng/mL and the y-axis is measured in days.

FIG. 7B is a plot of the mean (n=6) C3c urinary levels normalized to creatinine with 14-day Compound 1 treatment at defined timepoints of the Phase 2a study including screening, treatment, taper period and follow up. The x-axis is C3c/Cr urinary levels measured in μg/mmol and the y-axis is measured in days.

FIG. 7C is a plot of the mean (n=6) C3c urinary levels normalized to albumin with 14-day Compound 1 treatment at defined timepoints of the Phase 2a study including screening, treatment, taper period and follow up. The x-axis is C3c/Albumin urinary levels measured in ng/mg and the y-axis is measured in days.

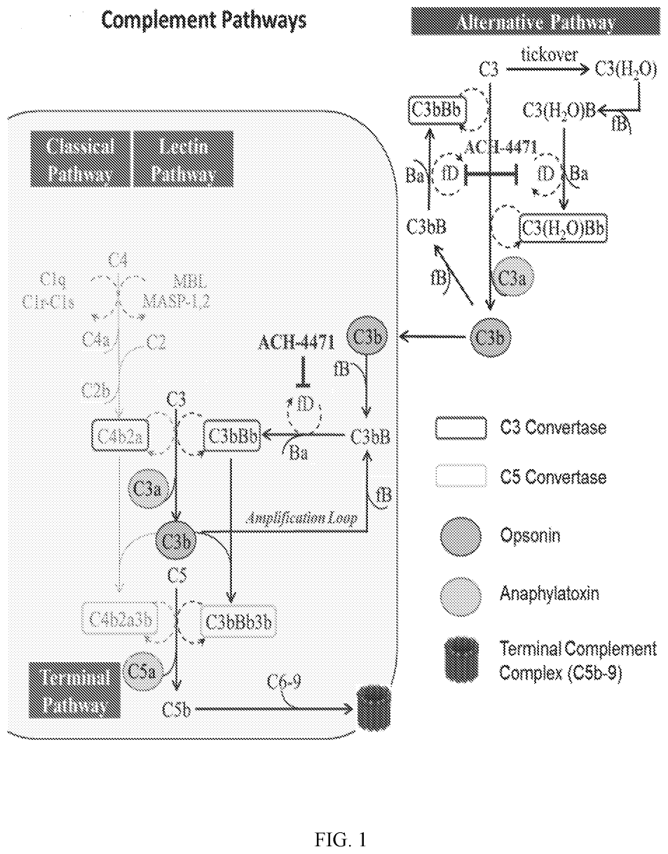

### DETAILED DESCRIPTION OF THE INVENTION

### Definitions

The terms “a” and “an” do not denote a limitation of quantity, but rather denote the presence of at least one of the referenced items. The term “or” means “and/or”. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of examples, or exemplary language (e.g., “such as”), is intended merely to better illustrate the invention and does not pose a limitation on the scope of the invention unless otherwise claimed. Unless defined otherwise, technical and scientific terms used herein have the same meaning as is commonly understood by one of skill in the art to which this invention belongs.

The “subject” treated is typically a human subject, although it is to be understood the methods described herein are effective with respect to other animals, such as mammals and vertebrate species. More particularly, the term “subject” can include animals used in assays such as those used in preclinical testing including but not limited to mice, rats, monkeys, dogs, pigs and rabbits; as well as domesticated swine (pigs and hogs), ruminants, equine, poultry, felines, bovines, murines, canines, and the like.

The term “pharmaceutically acceptable salt” as used herein refers to those salts which are, within the scope of sound medical judgment, suitable for use in contact with subjects (e.g., human subjects) without undue toxicity, irritation, allergic response, and the like, commensurate with a reasonable benefit/risk ratio, and effective for their intended use, as well as the zwitterionic forms, where possible, of the compounds of the presently disclosed subject matter.

Thus, the term “salt” refers to the relatively non-toxic, inorganic and organic acid addition salts of compounds of the presently disclosed subject matter. These salts can be prepared in situ during the final isolation and purification of the compounds or by separately reacting the purified Compound in its free base form with a suitable organic or inorganic acid and isolating the salt thus formed. Pharmaceutically acceptable base addition salts may be formed with metals or amines, such as alkali and alkaline earth metal hydroxides, or of organic amines. Examples of metals used as cations, include, but are not limited to, sodium, potassium, magnesium, calcium, and the like. Examples of suitable amines include, but are not limited to, N, N-dibenzylethylenediamine, chloroprocaine, choline, diethanolamine, ethylenediamine, N-methylglucamine, and procaine.

Salts can be prepared from inorganic acids sulfate, pyrosulfate, bisulfate, sulfite, bisulfite, nitrate, phosphate, monohydrogenphosphate, dihydrogenphosphate, metaphosphate, pyrophosphate, chloride, bromide, iodide such as hydrochloric, nitric, phosphoric, sulfuric, hydrobromic, hydriodic, phosphorus, and the like. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, laurylsulphonate and isethionate salts, and the like. Salts can also be prepared from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. and the like. Representative salts include acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Pharmaceutically acceptable salts can include cations based on the alkali and alkaline earth metals, such as sodium, lithium, potassium, calcium, magnesium and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. Also contemplated are the salts of amino acids such as arginate, gluconate, galacturonate, and the like. See, for example, Berge et al., J. Pharm. Sci., 1977, 66, 1-19, which is incorporated herein by reference.

“Pharmaceutical compositions” are compositions comprising at least one active agent, and at least one other substance, such as a pharmaceutically acceptable carrier or pharmaceutically acceptable excipient.

A “pharmaceutically acceptable excipient” means an excipient that is useful in preparing a pharmaceutical composition that is generally safe, non-toxic and neither biologically nor otherwise inappropriate for administration to a host, typically a human. In some embodiments, an excipient is used that is acceptable for veterinary use.

As used herein, the term “prodrug” means a compound which when administered to a host in vivo is converted into the parent drug. As used herein, the term “parent drug” means any of the presently described chemical compounds that are useful to treat any of the disorders described herein, or to control or improve the underlying cause or symptoms associated with any physiological or pathological disorder described herein in a host, typically a human. Prodrugs can be used to achieve any desired effect, including to enhance properties of the parent drug or to improve the pharmaceutic or pharmacokinetic properties of the parent. Prodrug strategies exist which provide choices in modulating the conditions for in vivo generation of the parent drug, all of which are deemed included herein. Nonlimiting examples of prodrug strategies include covalent attachment of removable groups, or removable portions of groups, for example, but not limited to acylation, phosphorylation, phosphonylation, phosphoramidate derivatives, amidation, reduction, oxidation, esterification, alkylation, other carboxy derivatives, sulfoxy or sulfone derivatives, carbonylation or anhydride, among others.

Throughout the specification and claims, a given chemical formula or name shall encompass all optical and stereoisomers, as well as racemic mixtures where such isomers and mixtures exist, unless otherwise noted.

In some embodiments, the compounds of Formula I or Formula II, or Compounds 1-25 include desired isotopic substitutions of atoms, at amounts above the natural abundance of the isotope, i.e., enriched. Isotopes are atoms having the same atomic number but different mass numbers, i.e., the same number of protons but a different number of neutrons. By way of general example and without limitation, isotopes of hydrogen, for example, deuterium (²H) and tritium (³H) may be used anywhere in described structures. Alternatively, or in addition, isotopes of carbon, e.g., ¹³C and ¹⁴C, may be used. A preferred isotopic substitution is deuterium for hydrogen at one or more locations on the molecule to improve the performance of the drug. The deuterium can be bound in a location of bond breakage during metabolism (an α-deuterium kinetic isotope effect) or next to or near the site of bond breakage (a (3-deuterium kinetic isotope effect).

Substitution with isotopes such as deuterium can afford certain therapeutic advantages resulting from greater metabolic stability, such as, for example, increased in vivo half-life or reduced dosage requirements. Substitution of deuterium for hydrogen at a site of metabolic break down can reduce the rate of, or eliminate, the metabolism at that bond. At any position of the compound that a hydrogen atom may be present, the hydrogen atom can be any isotope of hydrogen, including protium (¹H), deuterium (²H) and tritium (³H). Thus, reference herein to a compound encompasses all potential isotopic forms unless the context clearly dictates otherwise.

The term “isotopically-labeled” analog refers to an analog that is a “deuterated analog”, a “¹³C-labeled analog,” or a “deuterated/¹³C-labeled analog.” The term “deuterated analog” means a compound described herein, whereby a H-isotope, i.e., hydrogen/protium (¹H), is substituted by a H-isotope, i.e., deuterium (²H). Deuterium substitution can be partial or complete. Partial deuterium substitution means that at least one hydrogen is substituted by at least one deuterium. In certain embodiments, the isotope is 90%, 95%, or 99% or more enriched in an isotope at any location of interest. In some embodiments, it is deuterium that is 90%, 95%, or 99% enriched at a desired location.

In the description above, below, and herein generally, whenever any of the terms referring to Formula I, Formula II, or a specific compound, for example Compounds 1-25, are used, it should be understood that pharmaceutically acceptable salts, prodrugs, or compositions are considered included, unless otherwise stated or inconsistent with the text.

As contemplated herein and for purposes of the disclosed ranges herein, all ranges described herein include any and all numerical values occurring within the identified ranges. For example, a range of 1 to 10, or between 1 and 10, as contemplated herein, would include the numerical values 1, 2, 3, 4, 5, 6, 7, 8, 9, 10, as well as fractions thereof.

### Complement Alternative Pathway-Associated Nephropathies

The techniques and methods recited herein are used to treat a renal disorder involving activation of the complement alternative pathway. Examples of such disorders include: C3G; C3GN; dense deposit disease (DDD); IC-MPGN; atypical or typical hemolytic uremic syndrome (HUS); lupus nephritis resulting from systemic lupus erythematous (SLE); IgA nephropathy; anti-neutrophilic cytoplasmic autoantibody (ANCA) glomerulonephritis; scleroderma renal crisis; post-infectious glomerulonephritis; glomerulonephritis and vasculitis resulting from Henoch-Schonlein purpura (HSP); anti-glomerular basement membrane (GBM) or Goodpasture's disease; light chain deposition disease; contrast-induced nephropathy (CIN); membranous glomerulonephritis; cryoglobulinemia; pre-eclampsia and eclampsia; and minimal change disease.

In an alternative embodiment, the techniques and methods recited herein are used to treat a disorder of kidney transplantation such as delayed graft function (DGF), antibody-mediated rejection (AMR), and graft-versus-host disease (GvHD). Additionally, the techniques and methods recited herein may be used during kidney transplantation for patients at high risk of thrombus formation. In GvHD, donor T-cell activation leads to specific tissue damage. Interactions between the complement system and lymphocytes have been shown to regulate alloreactive T-cell and antigen-presenting cell (APC) function in the setting of allograft rejection. C3a and/or C5a may modulate T-cell-induced GvHD, and therefore targeting C3aR/C5aR signaling may be therapeutically efficacious. The complement pathway is also involved in the activation of donor APCs in host rejection.

Membranoproliferative glomerulonephritis (MPGN) is a disease that affects the glomeruli, or filters, of the kidneys. Until recently, MPGN was clinically classified as either primary, idiopathic MPGN or as secondary MPGN when an underlying etiology was identifiable. Primary MPGN was further classified into three types—type I, type II, and type III—based principally on the ultrastructural appearance and location of electron-dense deposits. Both the clinical and histopathologic schemes presented problems, however, as neither was based on disease pathogenesis. An improved understanding of the role of complement in the pathogenesis of MPGN has led to a proposed reclassification into immunoglobulin-mediated disease (driven by the classical complement pathway) and non-immunoglobulin-mediated disease (driven by the alternative complement pathway). This reclassification has led to improved diagnostic clinical algorithms and the emergence of a new grouping of diseases known as the C3 glomerulopathies, best represented by dense deposit disease and C3 glomerulonephritis.

C3 glomerulopathy is a group of related conditions that cause the kidneys to malfunction. The major features of C3 glomerulopathy include high levels of protein in the urine (proteinuria), blood in the urine (hematuria), reduced amounts of urine, low levels of protein in the blood, and swelling in many areas of the body. Affected individuals may have particularly low levels of complement component 3 (or C3) in the blood. The kidney problems associated with C3 glomerulopathy tend to worsen over time. About half of affected individuals develop end-stage renal disease (ESRD) within 10 years after their diagnosis. ESRD is a life-threatening condition that prevents the kidneys from filtering fluids and waste products from the body effectively.

Two major forms of C3 glomerulopathy have been identified: dense deposit disease (DDD) and C3 glomerulonephritis (C3GN). Although the two disorders cause similar kidney problems, the features of dense deposit disease tend to appear earlier than those of C3 glomerulonephritis, usually in adolescence. However, the signs and symptoms of either disease may not begin until adulthood.

Dense deposit disease can also be associated with other conditions unrelated to kidney function. For example, people with dense deposit disease may have acquired partial lipodystrophy, a condition characterized by a lack of fatty (adipose) tissue under the skin in the upper part of the body. Additionally, some people with dense deposit disease develop a buildup of yellowish deposits called drusen in the light-sensitive tissue at the back of the eye (the retina). These deposits usually appear in childhood or adolescence and can cause vision problems later in life.

Immune-complex membranoproliferative glomerulonephritis (IC-MPGN) is a renal disease which shares many clinical, pathologic, genetic and laboratory features with C3GN. Up to 40% of patients with IC-MPGN have no identifiable underlying etiology and are considered to have idiopathic IC-MPGN. Subjects with idiopathic IC-MPGN can have low C3 and normal C4 levels, similar to those observed in C3GN, as well as many of the same genetic or acquired factors that are associated with abnormal alternative pathway activity. Those subjects with low C3 and normal C4 levels are likely to have significant over-activity of the alternative pathway. The presence of C3 Nephrotic Factor (C3Nef) is identified as frequently in patients with MPGN type 1 as those with C3GN. Mutations in genes encoding the alternative pathway proteins including fH and fI are found in IC-MPGN patients. Despite immunopositive fluorescence staining for IgG, IgM, and C1q (in a fraction of cases) in kidney biopsy, approximately 46% of the IC-MPGN cases exhibit reduced C3 levels and normal C4. These data demonstrate that the alternative pathway is dysregulated in IC-MPGN.

Scleroderma is an autoimmune disorder that may result in changes to the skin, blood vessels, muscles, and internal organs and is typically characterized by areas of thickened skin, stiffness, feeling tired, and poor blood flow to the fingers and toes upon cold exposure. A major complication in patients with systemic sclerosis is scleroderma renal crisis (SRC), characterized by the presence of malignant hypertension and oliguric or anuric acute renal failure. The classical and alternative complement pathways have both been shown to be active during SRC.

Postinfectious glomerulonephritis (PIGN) results after an infectious by an organism, most commonly a nephritogenic strain of group A beta-hemolytic streptococci. The cause is unknown, but microbial antigens are believed to bind to the glomerular basement membrane and activation of the alternative complement pathway, leading to glomerular damage. Many recover completely from the prior infection, but those with prolonged PIGN often fail to terminate the alternative pathway after the infection has cleared. Mutations in complement regulating proteins or antibodies to the C3 convertase may be the cause. The sequelae are continual glomerular deposition of complement factors with resulting inflammation, leading to development of an ‘atypical’ PIGN.

Henoch-Schonlein purpura is a disease of the skin, mucous membranes, and other organs that causes palpable purpura (small raised areas of bleeding under the skin) combined with joint and abdominal pain. Kidney involvement may occur, leading to small amounts of hematuria and proteinuria, but typically not to a significant degree. In some cases, kidney involvement may progress to chronic kidney disease (glomerulonephritis). IgA aggregates or complexes with complement and is deposited in target organs, resulting in elaboration of inflammatory mediators. This may play a central role in the pathogenesis, as IgA complexes activate the complement alternative pathway. Complement pathway activation may enhance glomerular injury.

Anti-glomerular basement membrane (GBM) disease, also known as Goodpasture syndrome, is a rare autoimmune disease in which antibodies attack the basement membrane in the lungs and kidneys, leading to bleeding from the lungs and kidney failure. All complement pathways, including the alternative pathway, are involved in this disease. The classical and alternative pathways are activated in the kidneys of patients, and the alternative pathway may be particularly involved in the complement-induced damage that occurs.

Contrast-induced nephropathy (CIN) is the third most common cause of hospital-acquired acute renal injury and is a serious complication resulting from the administration of contrast media. The main proposed mechanism for the pathology of CIN involves the exacerbation of medullary hypoxia due to altered hemodynamics, which in the presence of impaired adaptive responses leads to tubular damage, and the direct cytotoxicity of the radiocontrast agents on tubular cells. Activation of the alternative complement pathway has been observed upon direct stimulation of endothelial cells after contrast media administration.

Membranous glomerulonephritis is an immune complex deposition disease that triggers complement pathway activation. Classical pathway activation is required to induce disease, but alternative pathway activation may also occur. Complement activation is essential to induce proteinuria.

Cryoglobulinemia is a medical condition in which the blood contains large amounts of cryoglobulins, proteins that become insoluble at reduced temperature. Cryoglobulinemia may result in a clinical syndrome of systemic inflammation that most commonly affects the kidneys and skin that is caused by cryoglobulin-containing immune complexes. The classical pathway is typically activated, but the alternative pathway may also be involved. Normal or low levels of component 3 (C3) and often undetectable levels of component 4 (C4) are observed.

Pre-eclampsia is a disorder of pregnancy characterized by the onset of high blood pressure and a significant proteinuria. In severe disease, kidney dysfunction may occur. The disorder may result in seizures if left untreated, at which point it is known as eclampsia. Excessive complement and maternal immune system activation in early pregnancy is suspected to be involved in the pathogenesis of pre-eclampsia. Bb levels have been found to be higher in maternal and umbilical venous blood in severe cases. Activation of the alternative complement pathway occurs both in the maternal and fetal compartments.

Minimal change disease is a nephrotic syndrome characterized by the loss of significant amounts of protein in the urine, leading to widespread edema and impaired kidney function. It is believed to be the result of an altered cell-mediated immune response that leads to increased glomerular permeability to serum albumin. Serum levels of Factors I and B were found to be lowered in those with active disease, while the serum level of C3 was found to be high.

### Alternative Pathway (AP) Inhibitors

In one aspect, the methods of the present invention contemplate the use of an inhibitor of the complement alternative pathway (AP). AP inhibitors are known in the art and may include, but are not limited to, complement factor D (fD) inhibitors, complement factor B (fB) inhibitors, complement factor H (fH) inhibitors, complement component 3 (C3) inhibitors, C3 convertase inhibitors, and C3b inhibitors.

In one aspect, the patient is administered a complement factor D (fD) inhibitor. Factor D inhibitors are known in the art, as described below. All patents and publications listed below describing fD inhibitors are hereby incorporated by reference.

In some embodiments, a fD inhibitor may be used as described by BioCryst Pharmaceuticals in U.S. Pat. No. 6,653,340, incorporated herein by reference, title “Compounds useful in the complement, coagulate and kallikrein pathways and methods for their preparation” which described fused bicyclic ring compounds that are potent inhibitors of Factor D.

In some embodiments, a fD inhibitor may be used as described by Novartis in PCT Patent Publication No. WO2012/093101 titled “Indole compounds or analogues thereof useful for the treatment of age-related macular degeneration,” incorporated herein by reference. In another embodiment, a fD inhibitor may be used as described in Novartis PCT Patent Publication Nos. WO2013/164802, WO2013/192345, WO2014/002051, WO2014/002052, WO2014/002053, WO2014/002054, WO2014/002057, WO2014/002058, WO2014/002059, WO2014/005150, WO2014/009833, WO2014/143638, WO2015/009616, WO2015/009977, or WO2015/066241, incorporated herein by reference.

In some embodiments, a fD inhibitor may be used as described by Bristol-Myers Squibb in PCT Patent Publication No. WO2004/045518 titled “Open chain prolyl urea-related modulators of androgen receptor function,” incorporated herein by reference.

In some embodiments, a fD inhibitor may be used as described by Japan Tobacco Inc. in PCT Patent Publication No. WO1999/048492 title “Amide derivatives and nociceptin antagonists,” incorporated herein by reference.

In some embodiments, a fD inhibitor may be used as described by Ferring B. V. and Yamanouchi Pharmaceutical Co. LTD. in PCT Patent Publication No. WO1993/020099 title “CCK and/or gastrin receptor ligands,” incorporated herein by reference.

In some embodiments, the fD inhibitor is the monoclonal antibody FCFD4515S as developed by Genentech/Roche.

In some embodiments, the fD inhibitor is Nafamostat (FUT-175, Futhan) as developed by Toni Pharmaceuticals.

In some embodiments, the fD inhibitor is aptamers (SOMAmers) as developed by SomaLogic.

In some embodiments, the fD inhibitor is the monoclonal antibody lampalizumab as developed by Roche.

In some embodiments, the fD inhibitor is aptamers to Factor D as developed by Vitrisa Therapeutics.

In some embodiments, the fD inhibitor is a fD inhibitor as developed by Ra Pharmaceuticals.

In another embodiment, the fD inhibitor comprises a drug disclosed in PCT/US17/014458.

In some embodiments, a fD inhibitor may be used as described by Alexion Pharmaceuticals in PCT Patent Publication No. WO1995/029697 title “Methods and compositions for the treatment of glomerulonephritis and other inflammatory diseases,” incorporated herein by reference.

In some embodiments, a fD inhibitor may be used as described by Achillion Pharmaceuticals in PCT Patent Application No. PCT/US2015/017523 and U.S. patent application Ser. No. 14/631,090 titled “Alkyne Compounds for Treatment of Complement Mediated Disorders”; PCT Patent Application No. PCT/US2015/017538 and U.S. patent application Ser. No. 14/631,233 title “Amide Compounds for Treatment of Complement Mediated Disorders”; PCT Patent Application No. PCT/US2015/017554 and U.S. patent application Ser. No. 14/631,312 titled “Amino Compounds for Treatment of Complement Mediated Disorders”; PCT Patent Application No. PCT/US2015/017583 and U.S. patent application Ser. No. 14/631,440 titled “Carbamate, Ester, and Ketone Compounds for Treatment of Complement Mediated Disorders”; PCT Patent Application No. PCT/US2015/017593 and U.S. patent application Ser. No. 14/631,625 titled “Aryl, Heteroaryl, and Heterocyclic Compounds for Treatment of Complement Mediated Disorders”; PCT Patent Application No. PCT/US2015/017597 and U.S. patent application Ser. No. 14/631,683 titled “Ether Compounds for Treatment of Complement Mediated Disorders”; PCT Patent Application No. PCT/US2015/017600 and U.S. patent application Ser. No. 14/631,785 titled “Phosphonate Compounds for Treatment of Complement Mediated Disorders”; or PCT Patent Application No. PCT/US2015/017609 and U.S. patent application Ser. No. 14/631,828 titled “Compounds for Treatment of Complement Mediated Disorders, incorporated herein by reference.

In some embodiments, a fD inhibitor of Formula I may be used:

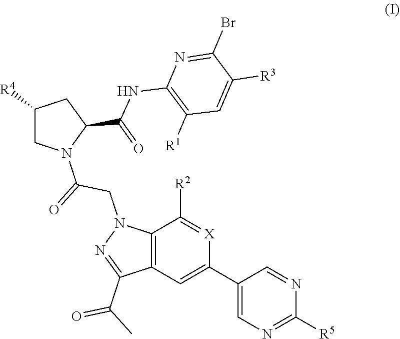

or a pharmaceutically acceptable salt, N-oxide, isotopic derivative, or prodrug thereof, optionally in a pharmaceutically acceptable carrier to form a composition;

wherein:

X is selected from N and CH;

R¹ is selected from hydrogen, C1-C3 alkyl, and halogen;

R² is selected from hydrogen and C1-C3 alkyl;

R³ is selected from hydrogen, C1-C3 alkyl, and halogen;

R⁴ is selected from hydrogen, C1-C3 alkyl, and halogen; and

R⁵ is selected from hydrogen, C1-C3 alkyl, halogen, and cyano.

In another embodiment, a fD inhibitor of Formula II may be used:

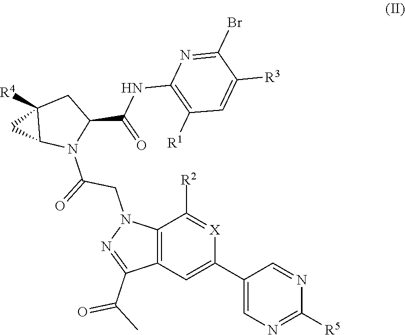

or a pharmaceutically acceptable salt, N-oxide, isotopic derivative, or prodrug thereof, optionally in a pharmaceutically acceptable carrier to form a composition;

wherein:

X is selected from N and CH;

R¹ is selected from hydrogen, C₁-C₃ alkyl, and halogen;

R² is selected from hydrogen and C₁-C₃ alkyl;

R³ is selected from hydrogen, C₁-C₃ alkyl, and halogen;

R¹ is selected from hydrogen, C₁-C₃ alkyl, and halogen; and

R⁵ is selected from hydrogen, C₁-C₃ alkyl, halogen, and cyano.

a. In some embodiments of any one of Formula I or Formula II, le is hydrogen.

b. In some embodiments of any one of Formula I or Formula II, le is C₁-C₃ alkyl.

c. In some embodiments of any one of Formula I or Formula II, le is methyl.

d. In some embodiments of any one of Formula I or Formula II, le is ethyl.

e. In some embodiments of any one of Formula I or Formula II, le is halogen.

f. Any one of embodiments a-e, wherein X is N.

g. Any one of embodiments a-e, wherein X is CH.

h. Any one of embodiments a-g, wherein R² is C₁-C₃ alkyl.

i. Any one of embodiments a-g, wherein R² is hydrogen.

j. Any one of embodiments a-g, wherein R² is methyl.

k. Any one of embodiments a-g, wherein R² is ethyl.

l. Any one of embodiments a-k, wherein R³ is hydrogen.

m. Any one of embodiments a-k, wherein R³ is methyl.

n. Any one of embodiments a-k, wherein R³ is ethyl.

o. Any one of embodiments a-k, wherein R³ is fluorine.

p. Any one of embodiments a-o, wherein R⁴ is C₁-C₃ alkyl.

q. Any one of embodiments a-o, wherein R⁴ is methyl.

r. Any one of embodiments a-o, wherein R⁴ is halogen.

s. Any one of embodiments a-r, wherein R⁵ is cyano.

t. Any one of embodiments a-r, wherein R⁵ is C₁-C₃ alkyl.

u. Any one of embodiments a-r, wherein R⁵ is methyl.

v. Any one of embodiments a-r, wherein R⁵ is halogen.

Compounds of Formula I and II can be synthesized using the methods disclosed in US20150239868 and US20170066783, incorporated herein by reference.

Non-limiting examples of compounds of Formula I and II are provided below in Table 1.

TABLE 1

| Cmpd | | |
|---|---|---|
| # | Structure | IUPAC Name |
| 1 | 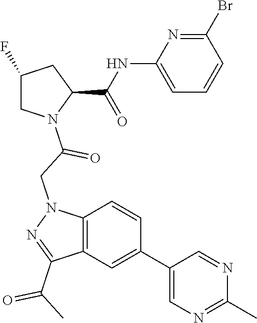 [SMILES: CC(=O)c1nn(CC(=O)N2C[C@H](F)C[C@H]2C(=O)Nc2cccc(Br)n2)c2ccc(-c3cnc(C)nc3)cc12] | (2S,4R)-1-(2-(3-acetyl-5-(2-methylpyrimidin-5- yl)-1H-indazol-1-yl)acetyl)-N-(6-bromopyridin-2- yl)-4-fluoropyrrolidine-2-carboxamide |
| 2 | 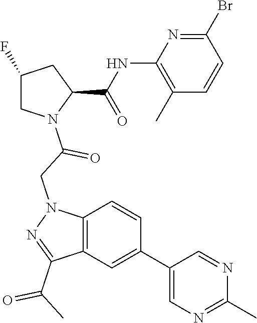 [SMILES: CC(=O)c1nn(CC(=O)N2C[C@H](F)C[C@H]2C(=O)Nc2nc(Br)ccc2C)c2ccc(-c3cnc(C)nc3)cc12] | (2S,4R)-1-(2-(3-acetyl-5-(2-methylpyrimidin-5- yl)-1H-indazol-1-yl)acetyl)-N-(6-bromo-3- methylpyridin-2-yl)-4-fluoropyrrolidine-2- carboxamide |
| 3 | 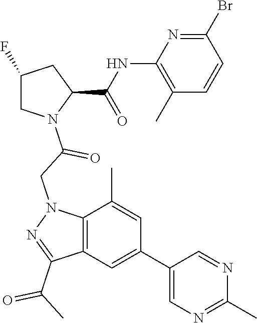 [SMILES: CC(=O)c1nn(CC(=O)N2C[C@H](F)C[C@H]2C(=O)Nc2nc(Br)ccc2C)c2c(C)cc(-c3cnc(C)nc3)cc12] | (2S,4R)-1-(2-(3-acetyl-7-methyl-5-(2- methylpyrimidin-5-yl)-1H-indazol-1-yl)acetyl)-N- (6-bromo-3-methylpyridin-2-yl)-4- fluoropyrrolidine-2-carboxamide |
| 4 | 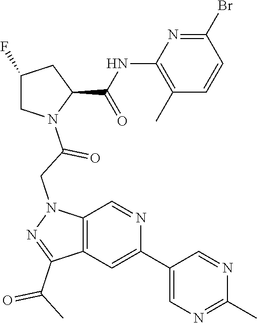 [SMILES: CC(=O)c1nn(CC(=O)N2C[C@H](F)C[C@H]2C(=O)Nc2nc(Br)ccc2C)c2cnc(-c3cnc(C)nc3)cc12] | (2S,4R)-1-(2-(3-acetyl-5-(2-methylpyrimidin-5- yl)-1H-pyrazolo[3,4-c]pyridin-1-yl)acetyl)-N-(6- bromo-3-methylpyridin-2-yl)-4-fluoropyrrolidine- 2-carboxamide |
| 5 | 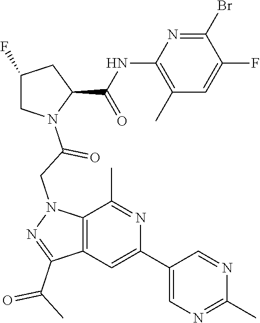 [SMILES: CC(=O)c1nn(CC(=O)N2C[C@H](F)C[C@H]2C(=O)Nc2nc(Br)c(F)cc2C)c2c(C)nc(-c3cnc(C)nc3)cc12] | (2S,4R)-1-(2-(3-acetyl-7-methyl-5-(2- methylpyrimidin-5-yl)-1H-pyrazolo[3,4-c]pyridin- 1-yl)acetyl)-N-(6-bromo-5-fluoro-3- methylpyridin-2-yl)-4-fluoropyrrolidine-2- carboxamide |
| 6 | 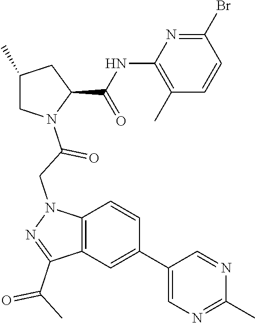 | (2S,4R)-1-(2-(3-acetyl-5-(2-methylpyrimidin-5- yl)-1H-indazol-1-yl)acetyl)-N-(6-bromo-3- methylpyridin-2-yl)-4-methylpyrrolidine-2- carboxamide |
| 7 | 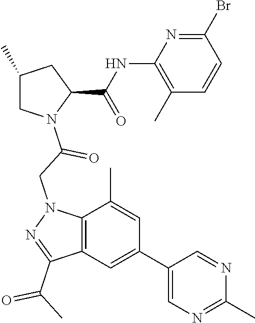 | (2S,4R)-1-(2-(3-acetyl-7-methyl-5-(2- methylpyrimidin-5-yl)-1H-indazol-1-yl)acetyl)-N- (6-bromo-3-methylpyridin-2-yl)-4- methylpyrrolidine-2-carboxamide |
| 8 | 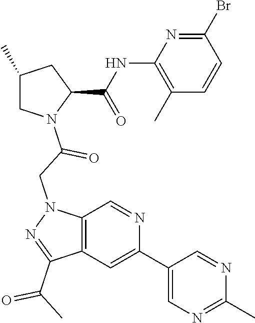 | (2S,4R)-1-(2-(3-acetyl-5-(2-methylpyrimidin-5- yl)-1H-pyrazolo[3,4-c]pyridin-1-yl)acetyl)-N-(6- bromo-3-methylpyridin-2-yl)-4-methylpyrrolidine- 2-carboxamide |
| 9 | 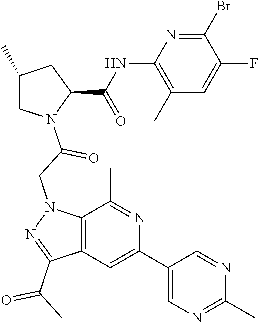 | (2S,4R)-1-(2-(3-acetyl-7-methyl-5-(2- methylpyrimidin-5-yl)-1H-pyrazolo[3,4-c]pyridin- 1-yl)acetyl)-N-(6-bromo-5-fluoro-3- methylpyridin-2-yl)-4-methylpyrrolidine-2- carboxamide |
| 10 | 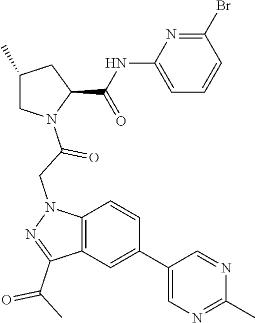 | (2S,4R)-1-(2-(3-acetyl-5-(2-methylpyrimidin-5- yl)-1H-indazol-1-yl)acetyl)-N-(6-bromopyridin-2- yl)-4-methylpyrrolidine-2-carboxamide |
| 11 | 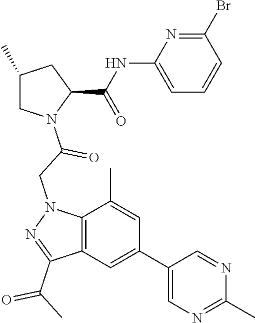 | (2S,4R)-1-(2-(3-acetyl-7-methyl-5-(2- methylpyrimidin-5-yl)-1H-indazol-1-yl)acetyl)-N- (6-bromopyridin-2-yl)-4-methylpyrrolidine-2- carboxamide |
| 12 | 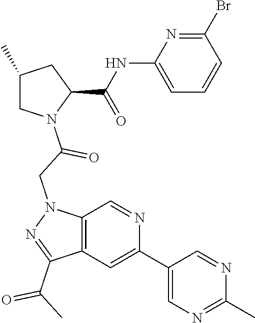 | (2S,4R)-1-(2-(3-acetyl-5-(2-methylpyrimidin-5- yl)-1H-pyrazolo[3,4-c]pyridin-1-yl)acetyl)-N-(6- bromopyridin-2-yl)-4-methylpyrrolidine-2- carboxamide |
| 13 | 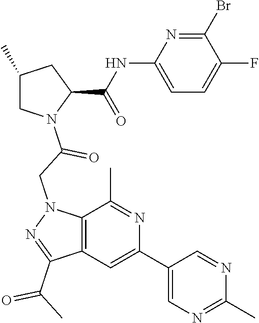 | (2S,4R)-1-(2-(3-acetyl-7-methyl-5-(2- methylpyrimidin-5-yl)-1H-pyrazolo[3,4-c]pyridin- 1-yl)acetyl)-N-(6-bromo-5-fluoropyridin-2-yl)-4- methylpyrrolidine-2-carboxamide |
| 14 | 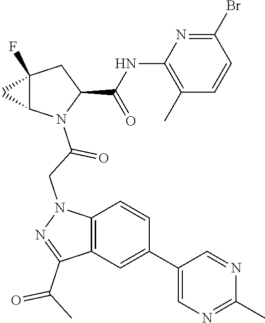 [SMILES: CC(=O)c1nn(CC(=O)N2[C@H](C(=O)Nc3nc(Br)ccc3C)C[C@@]3(F)C[C@@H]23)c2ccc(-c3cnc(C)nc3)cc12] | (1R,3S,5S)-2-(2-(3-acetyl-5-(2-methylpyrimidin- 5-yl)-1H-indazol-1-yl)acetyl)-N-(6-bromo-3- methylpyridin-2-yl)-5-fluoro-2- azabicyclo[3.1.0]hexane-3-carboxamide |
| 15 | 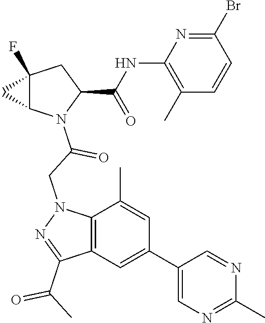 [SMILES: CC(=O)c1nn(CC(=O)N2[C@H](C(=O)Nc3nc(Br)ccc3C)C[C@@]3(F)C[C@@H]23)c2c(C)cc(-c3cnc(C)nc3)cc12] | (1R,3S,5S)-2-(2-(3-acetyl-7-methyl-5-(2- methylpyrimidin-5-yl)-1H-indazol-1-yl)acetyl)-N- (6-bromo-3-methylpyridin-2-yl)-5-fluoro-2- azabicyclo[3.1.0]hexane-3-carboxamide |
| 16 | 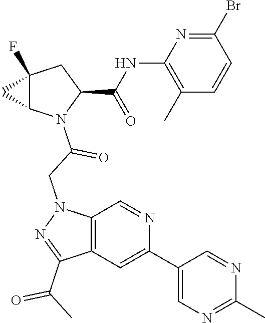 [SMILES: CC(=O)c1nn(CC(=O)N2[C@H](C(=O)Nc3nc(Br)ccc3C)C[C@@]3(F)C[C@@H]23)c2cnc(-c3cnc(C)nc3)cc12] | (1R,3S,5S)-2-(2-(3-acetyl-5-(2-methylpyrimidin- 5-yl)-1H-pyrazolo[3,4-c]pyridin-1-yl)acetyl)-N- (6-bromo-3-methylpyridin-2-yl)-5-fluoro-2- azabicyclo[3.1.0]hexane-3-carboxamide |
| 17 | 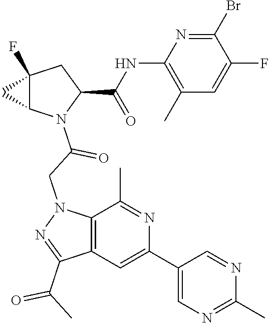 [SMILES: CC(=O)c1nn(CC(=O)N2[C@H](C(=O)Nc3nc(Br)c(F)cc3C)C[C@@]3(F)C[C@@H]23)c2c(C)nc(-c3cnc(C)nc3)cc12] | (1R,3S,5S)-2-(2-(3-acetyl-7-methyl-5-(2- methylpyrimidin-5-yl)-1H-pyrazolo[3,4-c]pyridin- 1-yl)acetyl)-N-(6-bromo-5-fluoro-3- methylpyridin-2-yl)-5-fluoro-2- azabicyclo[3.1.0]hexane-3-carboxamide |
| 18 | 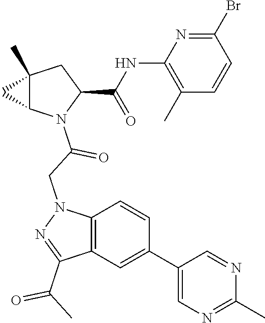 [SMILES: CC(=O)c1nn(CC(=O)N2[C@H](C(=O)Nc3nc(Br)ccc3C)C[C@@]3(C)C[C@@H]23)c2ccc(-c3cnc(C)nc3)cc12] | (1R,3S,5R)-2-(2-(3-acetyl-5-(2-methylpyrimidin- 5-yl)-1H-indazol-1-yl)acetyl)-N-(6-bromo-3- methylpyridin-2-yl)-5-methyl-2- azabicyclo[3.1.0]hexane-3-carboxamide |
| 19 | 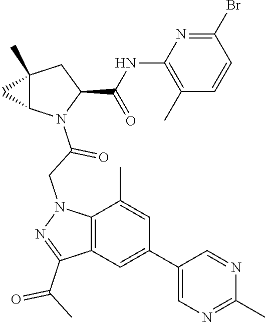 [SMILES: CC(=O)c1nn(CC(=O)N2[C@H](C(=O)Nc3nc(Br)ccc3C)C[C@@]3(C)C[C@@H]23)c2c(C)cc(-c3cnc(C)nc3)cc12] | (1R,3S,5R)-2-(2-(3-acetyl-7-methyl-5-(2- methylpyrimidin-5-yl)-1H-indazol-1-yl)acetyl)-N- (6-bromo-3-methylpyridin-2-yl)-5-methyl-2- azabicyclo[3.1.0]hexane-3-carboxamide |
| 20 | 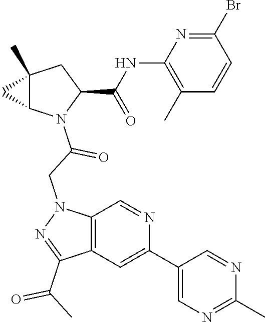 [SMILES: CC(=O)c1nn(CC(=O)N2[C@H](C(=O)Nc3nc(Br)ccc3C)C[C@@]3(C)C[C@@H]23)c2cnc(-c3cnc(C)nc3)cc12] | (1R,3S,5R)-2-(2-(3-acetyl-5-(2-methylpyrimidin- 5-yl)-1H-pyrazolo[3,4-c]pyridin-1-yl)acetyl)-N- (6-bromo-3-methylpyridin-2-yl)-5-methyl-2- azabicyclo[3.1.0]hexane-3-carboxamide |
| 21 | 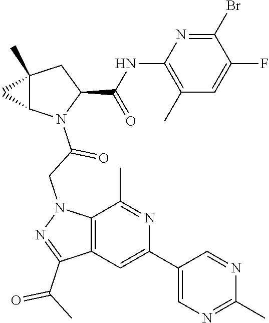 [SMILES: CC(=O)c1nn(CC(=O)N2[C@H](C(=O)Nc3nc(Br)c(F)cc3C)C[C@@]3(C)C[C@@H]23)c2c(C)nc(-c3cnc(C)nc3)cc12] | (1R,3S,5R)-2-(2-(3-acetyl-7-methyl-5-(2- methylpyrimidin-5-yl)-1H-pyrazolo[3,4-c]pyridin- 1-yl)acetyl)-N-(6-bromo-5-fluoro-3- methylpyridin-2-yl)-5-methyl-2- azabicyclo[3.1.0]hexane-3-carboxamide |
| 22 | 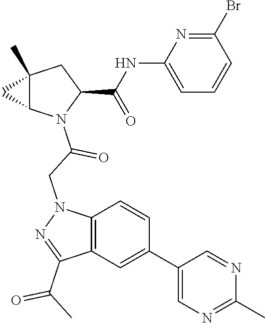 [SMILES: CC(=O)c1nn(CC(=O)N2[C@H](C(=O)Nc3cccc(Br)n3)C[C@@]3(C)C[C@@H]23)c2ccc(-c3cnc(C)nc3)cc12] | (1R,3S,5R)-2-(2-(3-acetyl-5-(2-methylpyrimidin- 5-yl)-1H-indazol-1-yl)acetyl)-N-(6-bromopyridin- 2-yl)-5-methyl-2-azabicyclo[3.1.0]hexane-3- carboxamide |
| 23 | 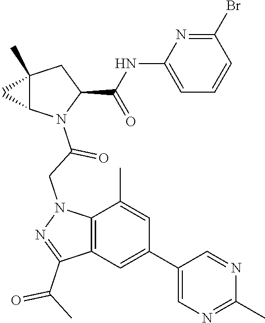 [SMILES: CC(=O)c1nn(CC(=O)N2[C@H](C(=O)Nc3cccc(Br)n3)C[C@@]3(C)C[C@@H]23)c2c(C)cc(-c3cnc(C)nc3)cc12] | (1R,3S,5R)-2-(2-(3-acetyl-7-methyl-5-(2- methylpyrimidin-5-yl)-1H-indazol-1-yl)acetyl)-N- (6-bromopyridin-2-yl)-5-methyl-2- azabicyclo[3.1.0]hexane-3-carboxamide |
| 24 | 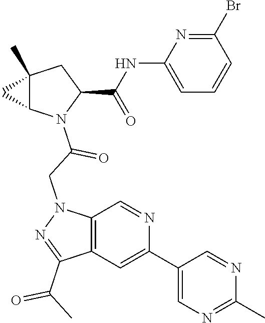 [SMILES: CC(=O)c1nn(CC(=O)N2[C@H](C(=O)Nc3cccc(Br)n3)C[C@@]3(C)C[C@@H]23)c2cnc(-c3cnc(C)nc3)cc12] | (1R,3S,5R)-2-(2-(3-acetyl-5-(2-methylpyrimidin- 5-yl)-1H-pyrazolo[3,4-c]pyridin-1-yl)acetyl)-N- (6-bromopyridin-2-yl)-5-methyl-2- azabicyclo[3.1.0]hexane-3-carboxamide |
| 25 | 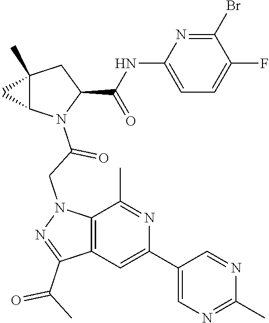 [SMILES: CC(=O)c1nn(CC(=O)N2[C@H](C(=O)Nc3ccc(F)c(Br)n3)C[C@@]3(C)C[C@@H]23)c2c(C)nc(-c3cnc(C)nc3)cc12] | (1R,3S,5R)-2-(2-(3-acetyl-7-methyl-5-(2- methylpyrimidin-5-yl)-1H-pyrazolo[3,4-c]pyridin- 1-yl)acetyl)-N-(6-bromo-5-fluoropyridin-2-yl)-5- methyl-2-azabicyclo[3.1.0]hexane-3-carboxamide |

An exemplary fD inhibitor for use in the present invention is, for example, Compound 1. Compound 1 is a potent Factor D inhibitor, having a binding affinity to human fD of K_{D}=0.54 nM, and an inhibition of catalytic activity of fD against Factor B of IC₅₀=17 nM. It also strongly inhibits AP activity in vitro, showing an IC₅₀ of 27 nM for rabbit erythrocyte hemolysis, 14 nM for PNH erythrocyte hemolysis, and 26 nM by Wieslab assay.

Methods of making Compound 1 are provided below:

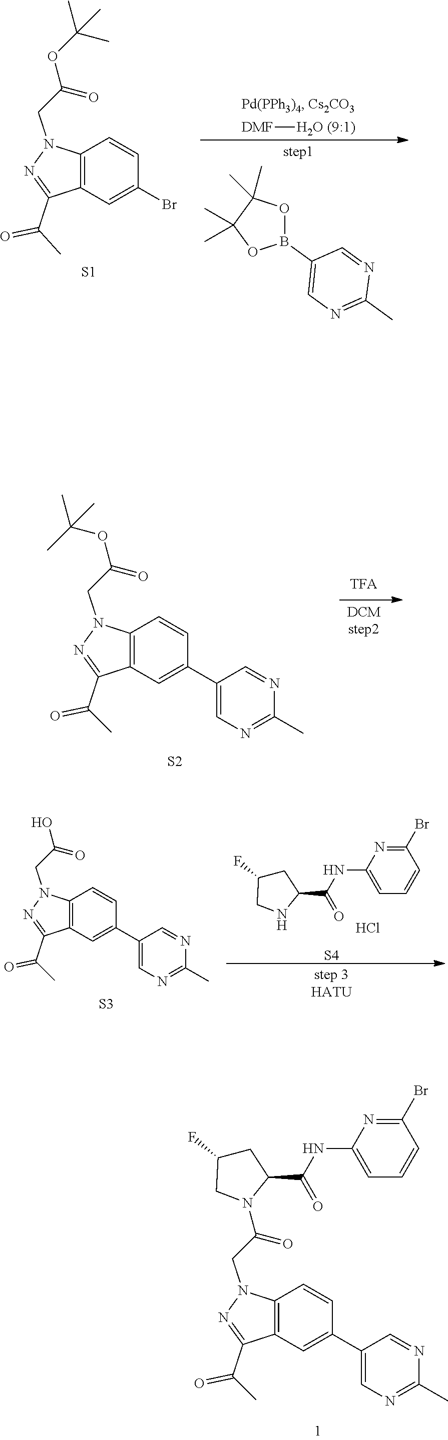

Factor D inhibitor ((2S,4R)-1-(2-(3-Acetyl-5-(2-methylpyrimidin-5-yl)-1H-indazol-1-yl) acetyl)-N-(6-bromopyridin-2-yl)-4-fluoropyrrolidine-2-carboxamide) (Compound 1) has been previously described, see U.S. Patent Appl. Pub. 2015/0239895 and 2017/0066783, incorporated herein by reference. Compound 1 may be synthesized by methods known to those in the art. In step 1, tert-butyl 2-(3-acetyl-5-bromo-1H-indazol-1-yl) acetate (S1) is coupled to 2-methyl-5-(4,4,5,5-tetramethyl-1,3,2-dioxaborolan-2-yl) pyrimidine using tetrakis(triphenylphosphine)palladium (0) in the presence of base to provide tert-butyl 2-(3-acetyl-5-(2-methylpyrimidin-5-yl)-1H-indazol-1-yl) acetate (S2). In step 2, hydrolysis of tert-butyl 2-(3-acetyl-5-(2-methylpyrimidin-5-yl)-1H-indazol-1-yl) acetate (S2) with trifluoroacetic acid provides 2-(3-acetyl-5-(2-methylpyrimidin-5-yl)-1H-indazol-1-yl) acetic acid (S3). In step 3, 2-(3-acetyl-5-(2-methylpyrimidin-5-yl)-1H-indazol-1-yl)acetic acid (S3) and (2S,4R)—N-(6-bromopyridin-2-yl)-4-fluoropyrrolidine-2-carboxamide (S4) are coupled using HATU to provide (2S,4R)-1-(2-(3-Acetyl-5-(2-methylpyrimidin-5-yl)-1H-indazol-1-yl)acetyl)-N-(6-bromopyridin-2-yl)-4-fluoropyrrolidine-2-carboxamide (1).

In another aspect, the patient is administered a Complement Factor B (fB) inhibitor. fB inhibitors are known in the art. Representative examples of fB inhibitors include, but are not limited to: anti-FB SiRNA (Alnylam Pharmaceuticals, Cambridge, Mass.); TA106 (monoclonal antibody, Alexion Pharmaceuticals, New Haven, Conn.); LNP023 (small molecule, Novartis, Basel, Switzerland); SOMAmers (aptamers, SomaLogic, Boulder, Colo.); bikaciomab (Novelmed Therapeutics, Cleveland, Ohio); complin (see, Kadam et al., J. Immunol. 2010, DOI:10.409/jimmunol.10000200); Ionis-FB-L_{Rx} (ligand conjugated antisense drug, Ionis Pharmaceuticals, Carlsbad, Calif.); or a combination thereof. In another embodiment, the fB inhibitor comprises a drug disclosed in PCT/US17/39587. In another embodiment, the fB inhibitor comprises a drug disclosed in U.S. Patent Appl. Pub. No. 2016/0024079 (assigned to Novartis AG). In some embodiments, the fB inhibitor is

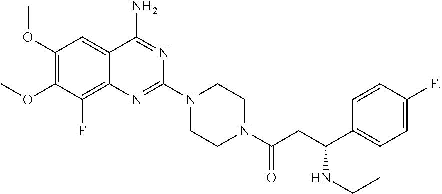 [SMILES: CCN[C@H](CC(=O)N1CCN(c2nc(N)c3cc(OC)c(OC)c(F)c3n2)CC1)c1ccc(F)cc1]

In some embodiments, the fB inhibitor is anti-FB siRNA. Anti-FB siRNA was developed by Alnylam Pharmaceuticals.

In some embodiments, the fB inhibitor is TA106. TA106 is a monoclonal antibody developed by Alexion Pharmaceuticals.

In some embodiments, the fB inhibitor is LNP023. LNP023 is a small molecule inhibitor of fB developed by Novartis. LNP023 and related inhibitors are described in Maibaum et al. Nat. Chem. Biol. 2016, 12:1105-1110.

In some embodiments, the fB inhibitor is complin. Complin is a peptide inhibitor that is described in Kadam et al. J. Immunol. 2010 184(12):7116-24.

In some embodiments, the fB inhibitor is Ionis-FB-L_{Rx}. Ionis-FB-L_{Rx} is a ligand conjugated antisense drug developed by Ionis Pharmaceuticals.

In some embodiments, the patient is administered a complement factor H (fH) inhibitor. fH inhibitors are known in the art. In some embodiments, the fH inhibitor is 5C6/AMY-301 (Amyndas). In some embodiments, the fH inhibitor is 5C6/composorbin (Amyndas).

In another aspect, the patient is administered a complement component 3 (C3) inhibitor. C3 inhibitors are known in the art. In some embodiments, the C3 inhibitor is compstatin and/or a compstatin analog. Compstatin and compstatin analogs are known and are found to be useful inhibitors of C3, see U.S. Pat. Nos. 9,056,076; 8,168,584; 9,421,240; 9,291,622; 8,580,735; 9,371,365; 9,169,307; 8,946,145; 7,989,589; 7,888,323; 6,319,897; and US Patent Appl. Pub. Nos. 2016/0060297; 2016/0015810; 2016/0215022; 2016/0215020; 2016/0194359; 2014/0371133; 2014/0323407; 2014/0050739; 2013/0324482; and 2015/0158915, incorporated herein by reference. In some embodiments, the C3 inhibitor is a compstatin analog. In some embodiments, the compstatin analog is 4(1MeW)/APL-1. In another embodiment, the compstatin analog is Cp40/AMY-101. In yet another embodiment, the compstatin analog is PEG-Cp40. In yet another embodiment, the compstatin analog is 4(1MeW) POT-4. 4(1MeW) POT-4 was developed by Potentia. In yet another embodiment, the compstatin analog is AMY-201. AMY-201 was developed by Amyndas Pharmaceuticals.

In some embodiments, the C3 inhibitor is selected from: H17 (monoclonal antibody, EluSys Therapeutics, Pine Brook, N.J.); mirococept (CR1-based protein); sCR1 (CR1-based protein, Celldex, Hampton, N.J.); TT32 (CR-1 based protein, Alexion Pharmaceuticals, New Haven, Conn.); HC-1496 (recombinant peptide); CB 2782 (enzyme, Catalyst Biosciences, South San Francisco, Calif.); APL-2 (pegylated synthetic cyclic peptide, Apellis Pharmaceuticals, Crestwood, Ky.); or combinations thereof.

In some embodiments, the C3 inhibitor is H17. H17 is a humanized monoclonal antibody in development by EluSys Therapeutics. H17 is described in Paixao-Cavalcante et al. J. Immunol. 2014, 192(10):4844-4851, incorporated herein by reference.

In some embodiments, the C3 inhibitor is mirococept. Mirococept is a CR1-based protein developed by Inflazyme Pharmaceuticals.

In some embodiments, the C3 inhibitor is sCR1. sCR1 is a soluble form of the CR1 protein developed by Celldex.

In some embodiments, the C3 inhibitor is TT32. TT32 is a CR-1 based protein developed by Alexion Pharmaceuticals.

In some embodiments, the C3 inhibitor is HC-1496. HC-1496 is a recombinant peptide developed by InCode.

In some embodiments, the C3 inhibitor is CB 2782. CB 2782 is novel protease derived from human membrane type serine protease 1 (MTSP-1) that was developed by Catalyst Biosciences.

In some embodiments, the C3 inhibitor is APL-2. APL-2 is a pegylated version of APL-1 developed by Apellis Pharmaceuticals.

In another aspect, the patient is administered a C3 convertase inhibitor. C3 convertase inhibitors are known in the art. In some embodiments, the C3 convertase inhibitor is CRIg/CFH. In some embodiments, the C3 convertase inhibitor is Mini-CFH (Amyndas). In some embodiments, the C3 convertase inhibitor is TT30 (CR2/CFH) (Alexion Pharmaceuticals). In some embodiments, the C3 convertase inhibitor is rFH (Optherion).

In another aspect, the patient is administered a C3b inhibitor. C3b inhibitors are known in the art. Some C3 inhibitors are also inhibitors of C3b, for example APL-2 (Apellis), 4(1MeW) POT-4 (Potential), PEG-Cp40 (Amyndas), and H17 (EluSys Therapeutics) described above. In some embodiments, the C3b inhibitor is ALXN1102/ALXN1103 (TT30) (Alexion Pharmaceuticals). In some embodiments, the C3b inhibitor is rFH (Optherion).

In some aspects, the AP inhibitor is a Bb inhibitor. In some embodiments, the Bb inhibitor is a monoclonal antibody to Bb.

### C5 Inhibitors

In some aspects of the present invention, the patient is currently or has previously been administered a C5 inhibitor. C5 inhibitors are known in the art. In some embodiments, the C5 inhibitor is a monoclonal antibody targeting C5. In some embodiments, the C5 inhibitor is eculizumab (Soliris™ Alexion Pharmaceuticals, New Haven, Conn., see, e.g., U.S. Pat. No. 9,352,035, incorporated herein by reference). In some embodiments, the C5 inhibitor is ravulizumab Cwvz (Ultomiris™ Alexion Pharmaceuticals, New Haven, Conn., see, e.g., U.S. Pat. Nos. 9,371,377; 9,079,949; and 9,663,574, incorporated herein by reference).

In some embodiments, the C5 inhibitor may be, but is not limited to: a recombinant human minibody, for example Mubodina® (monoclonal antibody, Adienne Pharma and Biotech, Bergamo, Italy; see U.S. Pat. No. 7,999,081, incorporated herein by reference); coversin (small animal protein, Volution Immuno-pharmaceuticals, Geneva, Switzerland; see e.g. Penabad et al. Lupus, 2012, 23(12):1324-6, incorporated herein by reference); LFG316 (monoclonal antibody, Novartis, Basel, Switzerland, and Morphosys, Planegg, Germany; see U.S. Pat. Nos. 8,241,628 and 8,883,158, each incorporated herein by reference); ARC-1905 (pegylated RNA aptamer, Iveric Bio, Princeton, N.J. and New York, N.Y.; see Keefe et al., Nature Reviews Drug Discovery, 9, 537-550, each of which is incorporated by reference); RA101348 and RA101495 (macrocyclic peptides, Ra Pharmaceuticals, Cambridge, Mass.); SOBI002 (affibody, Swedish Orphan Biovitrum, Stockholm, Sweden); ALN-CC5 (Si-RNA, Alnylam Pharmaceuticals, Cambridge, Mass.); ARC1005 (aptamers, Novo Nordisk, Bagsvaerd, Denmark); SOMAmers (aptamers, SomaLogic, Boulder, Co); SSL7 (bacterial protein toxin, see, e.g. Laursen et al. Proc. Natl. Acad. Sci. U.S.A., 107(8):3681-6, incorporated herein by reference); MEDI7814 (monoclonal antibody, MedImmune, Gaithersburg, Md.); aurin tricarboxylic acid; aurin tricarboxylic acid derivatives (Aurin Biotech, Vancouver, BC, see U.S. Patent Appl. Pub. 2013/003592, incorporated herein by reference); RG6107 (anti-C5 recycling antibody, Roche Pharmaceuticals, Basel, Switzerland); ALXN1210 and ALXN5500 (monoclonal antibodies, Alexion Pharmaceuticals, New Haven, Conn.); TT30 (fusion protein, Alexion Pharmaceuticals, New Haven, Conn.); REGN3918 (monoclonal antibody, Regeneron, Tarrytown, N.Y.); ABP959 (eculizumab biosimilar, Amgen, Thousand Oaks, Calif.); or combinations thereof.

In some embodiments, the C5 inhibitor is a recombinant human minibody, for example Mubodina®. Mubodina® is a fully human recombinant antibody C5 developed by Adienne Pharma and Biotech. Mubodina® is described in U.S. Pat. No. 7,999,081, incorporated herein by reference.

In some embodiments, the C5 inhibitor is coversin. Coversin is a recombinant protein derived from a protein discovered in the saliva of the Ornithodoros moubata tick currently developed as a recombinant protein by Akari Therapeutics. Coversin is described in Penabad et al. Lupus 2012, 23(12):1324-6, incorporated herein by reference.

In some embodiments, the C5 inhibitor is Tesidolumab/LFG316. Tesidolumab is a monoclonal antibody developed by Novartis and Morphosys. Tesidolumab is described in U.S. Pat. Nos. 8,241,628 and 8,883,158, incorporated herein by reference.

In some embodiments, the C5 inhibitor is ARC-1905. ARC-1905 is a pegylated RNA aptamer developed by Iveric Bio. ARC-1905 is described in Keefe et al. Nature Reviews Drug Discovery, 9:537-550, incorporated herein by reference.

In some embodiments, the C5 inhibitor is RA101348. RA101348 is a macrocyclic peptide developed by Ra Pharmaceuticals.

In some embodiments, the C5 inhibitor is RA101495. RA101495 is a macrocyclic peptide developed by Ra Pharmaceuticals.

In some embodiments, the C5 inhibitor is SOBI002. SOBI002 is an affibody developed by the Swedish Orphan Biovitrum.

In some embodiments, the C5 inhibitor is ARC1005. ARC1005 is an aptamer developed by Novo Nordisk.

In some embodiments, the C5 inhibitor is SOMAmers for C5. SOMAmers are aptamers developed by SomaLogic.

In some embodiments, the C5 inhibitor is SSL7. SSL7 is a bacterial protein toxin described in Laursen et al. Proc. Natl. Acad. Sci. U.S.A., 107(8):3681-6, incorporated herein by reference.

In some embodiments, the C5 inhibitor is MEDI7814. MEDI7814 is a monoclonal antibody developed by MedImmune.

In some embodiments, the C5 inhibitor is aurin tricarboxylic acid. In another embodiment, the C5 inhibitor is an aurin tricarboxylic acid derivative. These aurin derivatives were developed by Aurin Biotech and are further described in U.S. Patent Appl. Pub. No. 2013/003592, incorporated herein by reference).

In some embodiments, the C5 inhibitor is RG6107/SKY59. RG6107/SKY59 is an anti-05 recycling antibody developed by Roche Pharmaceuticals.

In some embodiments, the C5 inhibitor is ALXN5500. ALXN5500 is a monoclonal antibody developed by Alexion Pharmaceuticals.

In some embodiments, the C5 inhibitor is TT30. TT30 is a fusion protein developed by Alexion Pharmaceuticals.

In some embodiments, the C5 inhibitor is ABP959. ABP959 is an eculizumab biosimilar monoclonal antibody developed by Amgen.

In some embodiments, the C5 inhibitor is Anti-05 siRNA. Anti-05 siRNA was developed by Alnylam Pharmaceuticals.

In some embodiments, the C5 inhibitor is Erdigna®. Erdigna® is an antibody developed by Adienne Pharma.

In some embodiments, the C5 inhibitor is avacincaptad pegol/Zimura®. Avacincaptad pegol is in aptamer developed by Iveric Bio.

In some embodiments, the C5 inhibitor is SOBI005. SOBI005 is a protein in developed by the Swedish Orphan Biovitrum.

In some embodiments, the C5 inhibitor is ISU305. ISU305 is a monoclonal antibody developed by ISU ABXIS.

In some embodiments, the C5 inhibitor is REGN3918. REGN3918 is a monoclonal antibody developed by Regeneron.

### Methods of Treatment

In some embodiments, a method is provided for the targeted selection and treatment of a human subject suffering from a suspected AP-pathway associated nephropathy comprising: analyzing the level of Ba in the urine of the subject; comparing the subject's Ba urine level to a range of Ba urine levels derived from individuals that do not have a AP-pathway associated nephropathy (“normal Ba range”); and if the subject's Ba urine level is greater than the normal Ba range, administering to the subject an effective amount of a AP-inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from suspected C3GN comprising: analyzing the level of Ba in the urine of the subject; comparing the subject's Ba urine level to a range of Ba urine levels derived from individuals that do not have C3GN (“normal Ba range”); and if the subject's Ba urine level is greater than the normal Ba range, administering to the subject an effective amount of a AP-inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from suspected DDD comprising: analyzing the level of Ba in the urine of the subject; comparing the subject's Ba urine level to a range of Ba urine levels derived from individuals that do not have DDD (“normal Ba range”); and if the subject's Ba urine level is greater than the normal Ba range, administering to the subject an effective amount of an AP-inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from suspected IC-MPGN comprising: analyzing the level of Ba in the urine of the subject; comparing the subject's Ba urine level to a range of Ba urine levels derived from individuals that do not have IC-MPGN (“normal Ba range”); and if the subject's Ba urine level is greater than the normal Ba range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from a suspected AP-pathway associated nephropathy comprising: analyzing the level of sC5b-9 in the urine of the subject; comparing the subject's sC5b-9 urine level to a range of sC5b-9 urine levels derived from individuals that do not have a AP-pathway associated nephropathy (“normal sC5b-9 range”); and if the subject's sC5b-9 urine level is greater than the normal sC5b-9 range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from suspected C3GN comprising: analyzing the level of sC5b-9 in the urine of the subject; comparing the subject's sC5b-9 urine level to a range of sC5b-9 urine levels derived from individuals that do not have C3GN (“normal sC5b-9 range”); and if the subject's sC5b-9 urine level is greater than the normal sC5b-9 range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from suspected DDD comprising: analyzing the level of sC5b-9 in the urine of the subject; comparing the subject's sC5b-9 urine level to a range of sC5b-9 urine levels derived from individuals that do not have DDD (“normal sC5b-9 range”); and if the subject's sC5b-9 urine level is greater than the normal sC5b-9 range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from suspected IC-MPGN comprising: analyzing the level of sC5b-9 in the urine of the subject; comparing the subject's sC5b-9 urine level to a range of sC5b-9 urine levels derived from individuals that do not have IC-MPGN (“normal sC5b-9 range”); and if the subject's sC5b-9 urine level is greater than the normal sC5b-9 range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from a suspected AP-pathway associated nephropathy comprising: analyzing the level of C3c in the urine of the subject; comparing the subject's C3c urine level to a range of C3c urine levels derived from individuals that do not have a AP-pathway associated nephropathy (“normal C3c range”); and if the subject's C3c urine level is greater than the normal C3c range, administering to the subject an effective amount of an AP inhibitor, for example Compound 1, or a pharmaceutically acceptable salt thereof.

In some embodiments, a method is provided for the targeted selection and treatment of a human subject suffering from suspected C3GN comprising: analyzing the level of C3c in the urine of the subject; comparing the subject's C3c urine level to a range of C3c urine levels derived from individuals that do not have C3GN (“normal C3c range”); and if the subject's C3c urine level is greater than the normal C3c range, administering to the subject an effective amount of an AP inhibitor, for example Compound 1, or a pharmaceutically acceptable salt thereof.

In some embodiments, a method is provided for the targeted selection and treatment of a human subject suffering from suspected DDD comprising: analyzing the level of C3c in the urine of the subject; comparing the subject's C3c urine level to a range of C3c urine levels derived from individuals that do not have DDD (“normal C3c range”); and if the subject's C3c urine level is greater than the normal C3c range, administering to the subject an effective amount of an AP inhibitor, for example Compound 1, or a pharmaceutically acceptable salt thereof.

In some embodiments, a method is provided for the targeted selection and treatment of a human subject suffering from suspected IC-MPGN comprising: analyzing the level of C3c in the urine of the subject; comparing the subject's C3c urine level to a range of C3c urine levels derived from individuals that do not have IC-MPGN (“normal C3c range”); and if the subject's C3c urine level is greater than the normal C3c range, administering to the subject an effective amount of an AP inhibitor, for example Compound 1, or a pharmaceutically acceptable salt thereof.

In some embodiments, a method is provided for the targeted selection and treatment of a human subject suffering from a suspected AP-pathway associated nephropathy comprising: analyzing the levels of Ba and sC5b-9 in the urine of the subject; comparing the subject's Ba and sC5b-9 urine level to a range of Ba urine levels derived from individuals that do not have a AP-pathway associated nephropathy (“normal Ba range”) and a range of sC5b-9 urine levels derived from individuals that do not have a AP-pathway associated nephropathy (“normal sC5b-9 range”); and if the subject's Ba urine level is greater than the normal Ba range and/or the subject's SC5b-9 urine level is greater than the normal sC5b-9 range, administering to the subject an effective amount of AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from suspected C3GN comprising: analyzing the levels of Ba and sC5b-9 in the urine of the subject; comparing the subject's Ba and sC5b-9 urine level to a range of Ba urine levels derived from individuals that do not have C3GN (“normal Ba range”) and a range of sC5b-9 urine levels derived from individuals that do not have C3GN (“normal sC5b-9 range”); and if the subject's Ba urine level is greater than the normal Ba range and/or the subject's SC5b-9 urine level is greater than the normal sC5b-9 range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from suspected DDD comprising: analyzing the levels of Ba and sC5b-9 in the urine of the subject; comparing the subject's Ba and sC5b-9 urine level to a range of Ba urine levels derived from individuals that do not have DDD (“normal Ba range”) and a range of sC5b-9 urine levels derived from individuals that do not have DDD (“normal sC5b-9 range”); and if the subject's Ba urine level is greater than the normal Ba range and/or the subject's SC5b-9 urine level is greater than the normal sC5b-9 range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from suspected IC-MPGN comprising: analyzing the levels of Ba and sC5b-9 in the urine of the subject; comparing the subject's Ba and sC5b-9 urine level to a range of Ba urine levels derived from individuals that do not have IC-MPGN (“normal Ba range”) and a range of sC5b-9 urine levels derived from individuals that do not have IC-MPGN (“normal sC5b-9 range”); and if the subject's Ba urine level is greater than the normal Ba range and/or the subject's SC5b-9 urine level is greater than the normal sC5b-9 range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from a suspected AP-pathway associated nephropathy comprising: analyzing the levels of Ba and C3c in the urine of the subject; comparing the subject's Ba and C3c urine level to a range of Ba urine levels derived from individuals that do not have a AP-pathway associated nephropathy (“normal Ba range”) and a range of C3c urine levels derived from individuals that do not have a AP-pathway associated nephropathy (“normal C3c range”); and if the subject's Ba urine level is greater than the normal Ba range and/or the subject's C3c urine level is greater than the normal C3c range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from suspected C3GN comprising: analyzing the levels of Ba and C3c in the urine of the subject; comparing the subject's Ba and C3c urine level to a range of Ba urine levels derived from individuals that do not have C3GN (“normal Ba range”) and a range of C3c urine levels derived from individuals that do not have C3GN (“normal C3c range”); and if the subject's Ba urine level is greater than the normal Ba range and/or the subject's C3c urine level is greater than the normal C3c range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from suspected DDD comprising: analyzing the levels of Ba and C3c in the urine of the subject; comparing the subject's Ba and C3c urine level to a range of Ba urine levels derived from individuals that do not have DDD (“normal Ba range”) and a range of C3c urine levels derived from individuals that do not have DDD (“normal C3c range”); and if the subject's Ba urine level is greater than the normal Ba range and/or the subject's C3c urine level is greater than the normal C3c range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from suspected IC-MPGN comprising: analyzing the levels of Ba and C3c in the urine of the subject; comparing the subject's Ba and C3c urine level to a range of Ba urine levels derived from individuals that do not have IC-MPGN (“normal Ba range”) and a range of C3c urine levels derived from individuals that do not have IC-MPGN (“normal C3c range”); and if the subject's Ba urine level is greater than the normal Ba range and/or the subject's C3c urine level is greater than the normal C3c range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from a suspected AP-pathway associated nephropathy comprising: analyzing the levels of sC5b-9 and C3c in the urine of the subject; comparing the subject's sC5b-9 and C3c urine level to a range of sC5b-9 urine levels derived from individuals that do not have a AP-pathway associated nephropathy (“normal sC5b-9 range”) and a range of C3c urine levels derived from individuals that do not have a AP-pathway associated nephropathy (“normal C3c range”); and if the subject's sC5b-9 urine level is greater than the normal sC5b-9 range and/or the subject's C3c urine level is greater than the normal C3c range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from suspected C3GN comprising: analyzing the levels of sC5b-9 and C3c in the urine of the subject; comparing the subject's sC5b-9 and C3c urine level to a range of sC5b-9 urine levels derived from individuals that do not have C3GN (“normal sC5b-9 range”) and a range of C3c urine levels derived from individuals that do not have C3GN (“normal C3c range”); and if the subject's sC5b-9 urine level is greater than the normal sC5b-9 range and/or the subject's C3c urine level is greater than the normal C3c range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from suspected DDD comprising: analyzing the levels of sC5b-9 and C3c in the urine of the subject; comparing the subject's sC5b-9 and C3c urine level to a range of sC5b-9 urine levels derived from individuals that do not have DDD (“normal sC5b-9 range”) and a range of C3c urine levels derived from individuals that do not have DDD (“normal C3c range”); and if the subject's sC5b-9 urine level is greater than the normal sC5b-9 range and/or the subject's C3c urine level is greater than the normal C3c range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from suspected IC-MPGN comprising: analyzing the levels of sC5b-9 and C3c in the urine of the subject; comparing the subject's sC5b-9 and C3c urine level to a range of sC5b-9 urine levels derived from individuals that do not have IC-MPGN (“normal sC5b-9 range”) and a range of C3c urine levels derived from individuals that do not have IC-MPGN (“normal C3c range”); and if the subject's sC5b-9 urine level is greater than the normal sC5b-9 range and/or the subject's C3c urine level is greater than the normal C3c range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from a suspected AP-pathway associated nephropathy comprising: analyzing the levels of Ba, sC5b-9, and C3c in the urine of the subject; comparing the subject's Ba, sC5b-9, and C3c urine level to a range of Ba urine levels derived from individuals that do not have a AP-pathway associated nephropathy (“normal Ba range”), a range of sC5b-9 urine levels derived from individuals that do not have a AP-pathway associated nephropathy (“normal sC5b-9 range”), and a range of C3c urine levels derived from individuals that do not have a AP-pathway associated nephropathy (“normal C3c range”); and if the subject's Ba urine level is greater than the normal Ba range, the subject's sC5b-9 urine level is greater than the normal sC5b-9 range, and/or the subject's C3c urine level is greater than the normal C3c range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from suspected C3GN comprising: analyzing the levels of Ba, sC5b-9, and C3c in the urine of the subject; comparing the subject's Ba, sC5b-9, and C3c urine level to a range of Ba urine levels derived from individuals that do not have a C3GN (“normal Ba range”), a range of sC5b-9 urine levels derived from individuals that do not have a C3GN (“normal sC5b-9 range”), and a range of C3c urine levels derived from individuals that do not have a C3GN (“normal C3c range”); and if the subject's Ba urine level is greater than the normal Ba range, the subject's sC5b-9 urine level is greater than the normal sC5b-9 range, and/or the subject's C3c urine level is greater than the normal C3c range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from suspected DDD comprising: analyzing the levels of Ba, sC5b-9, and C3c in the urine of the subject; comparing the subject's Ba, sC5b-9, and C3c urine level to a range of Ba urine levels derived from individuals that do not have a DDD (“normal Ba range”), a range of sC5b-9 urine levels derived from individuals that do not have a DDD (“normal sC5b-9 range”), and a range of C3c urine levels derived from individuals that do not have a DDD (“normal C3c range”); and if the subject's Ba urine level is greater than the normal Ba range, the subject's sC5b-9 urine level is greater than the normal sC5b-9 range, and/or the subject's C3c urine level is greater than the normal C3c range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from suspected IC-MPGN comprising: analyzing the levels of Ba, sC5b-9, and C3c in the urine of the subject; comparing the subject's Ba, sC5b-9, and C3c urine level to a range of Ba urine levels derived from individuals that do not have a IC-MPGN (“normal Ba range”), a range of sC5b-9 urine levels derived from individuals that do not have a IC-MPGN (“normal sC5b-9 range”), and a range of C3c urine levels derived from individuals that do not have a IC-MPGN (“normal C3c range”); and if the subject's Ba urine level is greater than the normal Ba range, the subject's sC5b-9 urine level is greater than the normal sC5b-9 range, and/or the subject's C3c urine level is greater than the normal C3c range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In any of the above embodiments, the subject's Ba urine level is at least 2× higher than the upper limit of the normal Ba range. In any of the above embodiments, the subject's Ba urine level is at least 3× higher than the upper limit of the normal Ba range. In any of the above embodiments, the subject's Ba urine level is at least 4× higher than the upper limit of the normal Ba range. In any of the above embodiments, the subject's Ba urine level is at least 5× higher than the upper limit of the normal Ba range. In any of the above embodiments, the subject's Ba urine level is at least 6× higher than the upper limit of the normal Ba range. In any of the above embodiments, the subject's Ba urine level is at least 7× higher than the upper limit of the normal Ba range. In any of the above embodiments, the subject's Ba urine level is at least 8× higher than the upper limit of the normal Ba range. In any of the above embodiments, the subject's Ba urine level is at least 9× higher than the upper limit of the normal Ba range. In any of the above embodiments, the subject's Ba urine level is at least 10× higher than the upper limit of the normal Ba range. In any of the above embodiments, the subject's Ba urine level is at least 100× higher than the upper limit of the normal Ba range. In any of the above embodiments, the subject's Ba urine level is at least 200× higher than the upper limit of the normal Ba range. In any of the above embodiments, the subject's Ba urine level is at least 300× higher than the upper limit of the normal Ba range. In any of the above embodiments, the subject's Ba urine level is at least 400× higher than the upper limit of the normal Ba range. In any of the above embodiments, the subject's Ba urine level is at least 500× higher than the upper limit of the normal Ba range. In any of the above embodiments, the subject's Ba urine level is more than 500× higher than the upper limit of the normal Ba range.

In any of the above embodiments, the subject's sC5b-9 urine level is at least 2× higher than the upper limit of the normal sC5b-9 range. In any of the above embodiments, the subject's sC5b-9 urine level is at least 3× higher than the upper limit of the normal sC5b-9 range. In any of the above embodiments, the subject's sC5b-9 urine level is at least 4× higher than the upper limit of the normal sC5b-9 range. In any of the above embodiments, the subject's sC5b-9 urine level is at least 5× higher than the upper limit of the normal sC5b-9 range. In any of the above embodiments, the subject's sC5b-9 urine level is at least 6× higher than the upper limit of the normal sC5b-9 range. In any of the above embodiments, the subject's sC5b-9 urine level is at least 7× higher than the upper limit of the normal sC5b-9 range. In any of the above embodiments, the subject's sC5b-9 urine level is at least 8× higher than the upper limit of the normal sC5b-9 range. In any of the above embodiments, the subject's sC5b-9 urine level is at least 9× higher than the upper limit of the normal sC5b-9 range. In any of the above embodiments, the subject's sC5b-9 urine level is at least 10× higher than the upper limit of the normal sC5b-9 range. In any of the above embodiments, the subject's sC5b-9 urine level is at least 100× higher than the upper limit of the normal sC5b-9 range. In any of the above embodiments, the subject's sC5b-9 urine level is at least 200× higher than the upper limit of the normal sC5b-9 range. In any of the above embodiments, the subject's sC5b-9 urine level is at least 300× higher than the upper limit of the normal sC5b-9 range. In any of the above embodiments, the subject's sC5b-9 urine level is at least 400× higher than the upper limit of the normal sC5b-9 range. In any of the above embodiments, the subject's sC5b-9 urine level is at least 500× higher than the upper limit of the normal sC5b-9 range. In any of the above embodiments, the subject's sC5b-9 urine level is more than 500× higher than the upper limit of the normal sC5b-9 range.

In any of the above embodiments, the subject's C3c urine level is at least 2× higher than the upper limit of the normal C3c range. In any of the above embodiments, the subject's C3c urine level is at least 3× higher than the upper limit of the normal C3c range. In any of the above embodiments, the subject's C3c urine level is at least 4× higher than the upper limit of the normal C3c range. In any of the above embodiments, the subject's C3c urine level is at least 5× higher than the upper limit of the normal C3c range. In any of the above embodiments, the subject's C3c urine level is at least 6× higher than the upper limit of the normal C3c range. In any of the above embodiments, the subject's C3c urine level is at least 7× higher than the upper limit of the normal C3c range. In any of the above embodiments, the subject's C3c urine level is at least 8× higher than the upper limit of the normal C3c range. In any of the above embodiments, the subject's C3c urine level is at least 9× higher than the upper limit of the normal C3c range. In any of the above embodiments, the subject's C3c urine level is at least 10× higher than the upper limit of the normal C3c range. In any of the above embodiments, the subject's C3c urine level is at least 100× higher than the upper limit of the normal C3c range. In any of the above embodiments, the subject's C3c urine level is at least 200× higher than the upper limit of the normal C3c range. In any of the above embodiments, the subject's C3c urine level is at least 300× higher than the upper limit of the normal C3c range. In any of the above embodiments, the subject's C3c urine level is at least 400× higher than the upper limit of the normal C3c range. In any of the above embodiments, the subject's C3c urine level is at least 500× higher than the upper limit of the normal C3c range. In any of the above embodiments, the subject's C3c urine level is more than 500× higher than the upper limit of the normal C3c range.

In some embodiments, a method is provided for the targeted selection and treatment of a human subject suffering from a suspected AP-associated nephropathy comprising: analyzing the level of Ba in the urine of the subject; comparing the subject's Ba urine levels to a range of Ba urine levels derived from individuals that have an AP-associated nephropathy (“abnormal Ba range”); and if the subject's Ba urine level falls within the abnormal Ba range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from suspected C3GN comprising: analyzing the level of Ba in the urine of the subject; comparing the subject's Ba urine levels to a range of Ba urine levels derived from individuals that have C3GN (“C3GN Ba range”); and if the subject's Ba urine level falls within the C3GN Ba range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from suspected DDD comprising: analyzing the level of Ba in the urine of the subject; comparing the subject's Ba urine levels to a range of Ba urine levels derived from individuals that have DDD (“DDD Ba range”); and if the subject's Ba urine level falls within the DDD Ba range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from suspected IC-MPGN comprising: analyzing the level of Ba in the urine of the subject; comparing the subject's Ba urine levels to a range of Ba urine levels derived from individuals that have IC-MPGN (“IC-MPGN Ba range”); and if the subject's Ba urine level falls within the IC-MPGN Ba range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from a suspected AP-associated nephropathy comprising: analyzing the level of C3c in the urine of the subject; comparing the subject's C3c urine levels to a range of C3c urine levels derived from individuals that have an AP-associated nephropathy (“abnormal C3c range”); and if the subject's C3c urine level falls within the abnormal C3c range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from suspected C3GN comprising: analyzing the level of C3c in the urine of the subject; comparing the subject's C3c urine levels to a range of C3c urine levels derived from individuals that have C3GN (“C3GN C3c range”); and if the subject's C3c urine level falls within the C3GN C3c range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from suspected DDD comprising: analyzing the level of C3c in the urine of the subject; comparing the subject's C3c urine levels to a range of C3c urine levels derived from individuals that have DDD (“DDD C3c range”); and if the subject's C3c urine level falls within the DDD C3c range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from suspected IC-MPGN comprising: analyzing the level of C3c in the urine of the subject; comparing the subject's C3c urine levels to a range of C3c urine levels derived from individuals that have IC-MPGN (“IC-MPGN C3c range”); and if the subject's C3c urine level falls within the IC-MPGN C3c range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from a suspected AP-associated nephropathy comprising: analyzing the levels of Ba and sC5b-9 in the urine of the subject; comparing the subject's Ba urine levels to a range of Ba urine levels derived from individuals that have an AP-associated nephropathy (“abnormal Ba range”) and the subject's sC5b-9 urine levels to a range of sC5b-9 urine levels derived from individuals that have an AP-associated nephropathy (“abnormal sC5b-9 range”); and if the subject's Ba urine level falls within the abnormal Ba range and/or the subject's sC5b-9 urine level falls within the abnormal sC5b-9 range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from suspected C3GN comprising: analyzing the levels of Ba and sC5b-9 in the urine of the subject; comparing the subject's Ba urine levels to a range of Ba urine levels derived from individuals that have C3GN (“C3GN Ba range”) and the subject's sC5b-9 urine levels to a range of sC5b-9 urine levels derived from individuals that have C3GN (“C3GN sC5b-9 range”); and if the subject's Ba urine level falls within the C3GN Ba range and/or the subject's sC5b-9 urine level falls within the C3GN sC5b-9 range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from suspected DDD comprising: analyzing the levels of Ba and sC5b-9 in the urine of the subject; comparing the subject's Ba urine levels to a range of Ba urine levels derived from individuals that have DDD (“DDD Ba range”) and the subject's sC5b-9 urine levels to a range of sC5b-9 urine levels derived from individuals that have DDD (“DDD sC5b-9 range”); and if the subject's Ba urine level falls within the DDD Ba range and/or the subject's sC5b-9 urine level falls within the DDD sC5b-9 range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from suspected IC-MPGN comprising: analyzing the levels of Ba and sC5b-9 in the urine of the subject; comparing the subject's Ba urine levels to a range of Ba urine levels derived from individuals that have IC-MPGN (“IC-MPGN Ba range”) and the subject's sC5b-9 urine levels to a range of sC5b-9 urine levels derived from individuals that have IC-MPGN (“IC-MPGN sC5b-9 range”); and if the subject's Ba urine level falls within the IC-MPGN Ba range and/or the subject's sC5b-9 urine level falls within the IC-MPGN sC5b-9 range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from a suspected AP-associated nephropathy comprising: analyzing the levels of Ba and C3c in the urine of the subject; comparing the subject's Ba urine levels to a range of Ba urine levels derived from individuals that have an AP-associated nephropathy (“abnormal Ba range”) and the subject's C3c urine levels to a range of C3c urine levels derived from individuals that have an AP-associated nephropathy (“abnormal C3c range”); and if the subject's Ba urine level falls within the abnormal Ba range and/or the subject's C3c urine level falls within the abnormal C3c range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from suspected C3GN comprising: analyzing the levels of Ba and C3c in the urine of the subject; comparing the subject's Ba urine levels to a range of Ba urine levels derived from individuals that have C3GN (“C3GN Ba range”) and the subject's C3c urine levels to a range of C3c urine levels derived from individuals that have C3GN (“C3GN C3c range”); and if the subject's Ba urine level falls within the C3GN Ba range and/or the subject's C3c urine level falls within the C3GN C3c range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from suspected DDD comprising: analyzing the levels of Ba and C3c in the urine of the subject; comparing the subject's Ba urine levels to a range of Ba urine levels derived from individuals that have DDD (“DDD Ba range”) and the subject's C3c urine levels to a range of C3c urine levels derived from individuals that have DDD (“DDD C3c range”); and if the subject's Ba urine level falls within the DDD Ba range and/or the subject's C3c urine level falls within the DDD C3c range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from suspected IC-MPGN comprising: analyzing the levels of Ba and C3c in the urine of the subject; comparing the subject's Ba urine levels to a range of Ba urine levels derived from individuals that have IC-MPGN (“IC-MPGN Ba range”) and the subject's C3c urine levels to a range of C3c urine levels derived from individuals that have IC-MPGN (“IC-MPGN C3c range”); and if the subject's Ba urine level falls within the IC-MPGN Ba range and/or the subject's C3c urine level falls within the IC-MPGN C3c range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from a suspected AP-associated nephropathy comprising: analyzing the levels of sC5b-9 and C3c in the urine of the subject; comparing the subject's sC5b-9 urine levels to a range of sC5b-9 urine levels derived from individuals that have an AP-associated nephropathy (“abnormal sC5b-9 range”) and the subject's C3c urine levels to a range of C3c urine levels derived from individuals that have an AP-associated nephropathy (“abnormal C3c range”); and if the subject's sC5b-9 urine level falls within the abnormal sC5b-9 range and/or the subject's C3c urine level falls within the abnormal C3c range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from suspected C3GN comprising: analyzing the levels of sC5b-9 and C3c in the urine of the subject; comparing the subject's sC5b-9 urine levels to a range of sC5b-9 urine levels derived from individuals that have C3GN (“C3GN sC5b-9 range”) and the subject's C3c urine levels to a range of C3c urine levels derived from individuals that have C3GN (“C3GN C3c range”); and if the subject's sC5b-9 urine level falls within the C3GN sC5b-9 range and/or the subject's C3c urine level falls within the C3GN C3c range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from suspected DDD comprising: analyzing the levels of sC5b-9 and C3c in the urine of the subject; comparing the subject's sC5b-9 urine levels to a range of sC5b-9 urine levels derived from individuals that have DDD (“DDD sC5b-9 range”) and the subject's C3c urine levels to a range of C3c urine levels derived from individuals that have DDD (“DDD C3c range”); and if the subject's sC5b-9 urine level falls within the DDD sC5b-9 range and/or the subject's C3c urine level falls within the DDD C3c range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from suspected IC-MPGN comprising: analyzing the levels of sC5b-9 and C3c in the urine of the subject; comparing the subject's sC5b-9 urine levels to a range of sC5b-9 urine levels derived from individuals that have IC-MPGN (“IC-MPGN sC5b-9 range”) and the subject's C3c urine levels to a range of C3c urine levels derived from individuals that have IC-MPGN (“IC-MPGN C3c range”); and if the subject's sC5b-9 urine level falls within the IC-MPGN sC5b-9 range and/or the subject's C3c urine level falls within the IC-MPGN C3c range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from a suspected AP-associated nephropathy comprising: analyzing the levels of Ba, sC5b-9, and C3c in the urine of the subject; comparing the subject's Ba urine levels to a range of Ba urine levels derived from individuals that have an AP-associated nephropathy (“abnormal Ba range”), the subject's sC5b-9 urine levels to a range of sC5b-9 urine levels derived from individuals that have an AP-associated nephropathy (“abnormal sC5b-9 range”), and the subject's C3c urine levels to a range of C3c urine levels derived from individuals that have an AP-associated nephropathy (“abnormal C3c range”); and if the subject's Ba urine level falls within the abnormal Ba range, the subject's sC5b-9 urine level falls within the abnormal sC5b-9 range and/or the subject's C3c urine level falls within the abnormal C3c range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from suspected C3GN comprising: analyzing the levels of Ba, sC5b-9, and C3c in the urine of the subject; comparing the subject's Ba urine levels to a range of Ba urine levels derived from individuals that have C3GN (“C3GN Ba range”), the subject's sC5b-9 urine levels to a range of sC5b-9 urine levels derived from individuals that have C3GN (“C3GN sC5b-9 range”), and the subject's C3c urine levels to a range of C3c urine levels derived from individuals that have C3GN (“C3GN C3c range”); and if the subject's Ba urine level falls within the C3GN Ba range, the subject's sC5b-9 urine level falls within the C3GN sC5b-9 range and/or the subject's C3c urine level falls within the C3GN C3c range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from suspected DDD comprising: analyzing the levels of Ba, sC5b-9, and C3c in the urine of the subject; comparing the subject's Ba urine levels to a range of Ba urine levels derived from individuals that have DDD (“DDD Ba range”), the subject's sC5b-9 urine levels to a range of sC5b-9 urine levels derived from individuals that have DDD (“DDD sC5b-9 range”), and the subject's C3c urine levels to a range of C3c urine levels derived from individuals that have DDD (“DDD C3c range”); and if the subject's Ba urine level falls within the DDD Ba range, the subject's sC5b-9 urine level falls within the DDD sC5b-9 range and/or the subject's C3c urine level falls within the DDD C3c range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method is provided for the targeted selection and treatment of a human subject suffering from suspected IC-MPGN comprising: analyzing the levels of Ba, sC5b-9, and C3c in the urine of the subject; comparing the subject's Ba urine levels to a range of Ba urine levels derived from individuals that have IC-MPGN (“IC-MPGN Ba range”), the subject's sC5b-9 urine levels to a range of sC5b-9 urine levels derived from individuals that have IC-MPGN (“IC-MPGN sC5b-9 range”), and the subject's C3c urine levels to a range of C3c urine levels derived from individuals that have IC-MPGN (“IC-MPGN C3c range”); and if the subject's Ba urine level falls within the IC-MPGN Ba range, the subject's sC5b-9 urine level falls within the IC-MPGN sC5b-9 range and/or the subject's C3c urine level falls within the IC-MPGN C3c range, administering to the subject an effective amount of an AP inhibitor. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method of monitoring the efficacy of a an AP inhibitor treatment regimen in a human subject having an AP-associated nephropathy and receiving an AP inhibitor is provided comprising: analyzing the Ba level in a first urine sample from the subject; analyzing the Ba level in a subsequent urine sample from the subject, wherein the first urine sample is collected prior to the subsequent urine sample; comparing the Ba level in the first urine sample to the Ba level in the subsequent urine sample from the subject; and if the Ba level in the subsequent urine sample is equal to or greater than the Ba level in the first urine sample, then increasing the dose of the AP inhibitor administered to the subject. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method of monitoring the efficacy of an AP inhibitor treatment regimen in a human subject having C3GN and receiving an AP inhibitor is provided comprising: analyzing the Ba level in a first urine sample from the subject; analyzing the Ba level in a subsequent urine sample from the subject, wherein the first urine sample is collected prior to the subsequent urine sample; comparing the Ba level in the first urine sample to the Ba level in the subsequent urine sample from the subject; and if the Ba level in the subsequent urine sample is equal to or greater than the Ba level in the first urine sample, then increasing the dose of the AP inhibitor administered to the subject. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method of monitoring the efficacy of an AP inhibitor treatment regimen in a human subject having DDD and receiving an AP inhibitor is provided comprising: analyzing the Ba level in a first urine sample from the subject; analyzing the Ba level in a subsequent urine sample from the subject, wherein the first urine sample is collected prior to the subsequent urine sample; comparing the Ba level in the first urine sample to the Ba level in the subsequent urine sample from the subject; and if the Ba level in the subsequent urine sample is equal to or greater than the Ba level in the first urine sample, then increasing the dose of the AP inhibitor administered to the subject. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method of monitoring the efficacy of an AP inhibitor treatment regimen in a human subject having IC-MPGN and receiving Compound 1 is provided comprising: analyzing the Ba level in a first urine sample from the subject; analyzing the Ba level in a subsequent urine sample from the subject, wherein the first urine sample is collected prior to the subsequent urine sample; comparing the Ba level in the first urine sample to the Ba level in the subsequent urine sample from the subject; and if the Ba level in the subsequent urine sample is equal to or greater than the Ba level in the first urine sample, then increasing the dose of the AP inhibitor administered to the subject. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method of monitoring the efficacy of an AP inhibitor treatment regimen in a human subject having an AP-associated nephropathy and receiving an AP inhibitor is provided comprising: analyzing the sC5b-9 level in a first urine sample from the subject; analyzing the sC5b-9 level in a subsequent urine sample from the subject, wherein the first urine sample is collected prior to the subsequent urine sample; comparing the sC5b-9 level in the first urine sample to the sC5b-9 level in the subsequent urine sample from the subject; and if the sC5b-9 level in the subsequent urine sample is equal to or greater than the sC5b-9 level in the first urine sample, then increasing the dose of the AP inhibitor administered to the subject. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method of monitoring the efficacy of an AP inhibitor treatment regimen in a human subject having C3GN and receiving an AP inhibitor is provided comprising: analyzing the sC5b-9 level in a first urine sample from the subject; analyzing the sC5b-9 level in a subsequent urine sample from the subject, wherein the first urine sample is collected prior to the subsequent urine sample; comparing the sC5b-9 level in the first urine sample to the sC5b-9 level in the subsequent urine sample from the subject; and if the sC5b-9 level in the subsequent urine sample is equal to or greater than the sC5b-9 level in the first urine sample, then increasing the dose of the AP inhibitor administered to the subject. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method of monitoring the efficacy of an AP inhibitor treatment regimen in a human subject having DDD and receiving an AP inhibitor is provided comprising: analyzing the sC5b-9 level in a first urine sample from the subject; analyzing the sC5b-9 level in a subsequent urine sample from the subject, wherein the first urine sample is collected prior to the subsequent urine sample; comparing the sC5b-9 level in the first urine sample to the sC5b-9 level in the subsequent urine sample from the subject; and if the sC5b-9 level in the subsequent urine sample is equal to or greater than the sC5b-9 level in the first urine sample, then increasing the dose of the AP inhibitor administered to the subject. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method of monitoring the efficacy of an AP inhibitor treatment regimen in a human subject having IC-MPGN and receiving an AP inhibitor is provided comprising: analyzing the sC5b-9 level in a first urine sample from the subject; analyzing the sC5b-9 level in a subsequent urine sample from the subject, wherein the first urine sample is collected prior to the subsequent urine sample; comparing the sC5b-9 level in the first urine sample to the sC5b-9 level in the subsequent urine sample from the subject; and if the sC5b-9 level in the subsequent urine sample is equal to or greater than the sC5b-9 level in the first urine sample, then increasing the dose of the AP inhibitor administered to the subject. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method of monitoring the efficacy of an AP inhibitor treatment regimen in a human subject having an AP-associated nephropathy and receiving an AP inhibitor is provided comprising: analyzing the C3c level in a first urine sample from the subject; analyzing the C3c level in a subsequent urine sample from the subject, wherein the first urine sample is collected prior to the subsequent urine sample; comparing the C3c level in the first urine sample to the C3c level in the subsequent urine sample from the subject; and if the C3c level in the subsequent urine sample is equal to or greater than the C3c level in the first urine sample, then increasing the dose of the AP inhibitor administered to the subject. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method of monitoring the efficacy of an AP inhibitor treatment regimen in a human subject having C3GN and receiving an AP inhibitor is provided comprising: analyzing the C3c level in a first urine sample from the subject; analyzing the C3c level in a subsequent urine sample from the subject, wherein the first urine sample is collected prior to the subsequent urine sample; comparing the C3c level in the first urine sample to the C3c level in the subsequent urine sample from the subject; and if the C3c level in the subsequent urine sample is equal to or greater than the C3c level in the first urine sample, then increasing the dose of the AP inhibitor administered to the subject. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method of monitoring the efficacy of an AP inhibitor treatment regimen in a human subject having DDD and receiving an AP inhibitor is provided comprising: analyzing the C3c level in a first urine sample from the subject; analyzing the C3c level in a subsequent urine sample from the subject, wherein the first urine sample is collected prior to the subsequent urine sample; comparing the C3c level in the first urine sample to the C3c level in the subsequent urine sample from the subject; and if the C3c level in the subsequent urine sample is equal to or greater than the C3c level in the first urine sample, then increasing the dose of the AP inhibitor administered to the subject. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method of monitoring the efficacy of an AP inhibitor treatment regimen in a human subject having IC-MPGN and receiving an AP inhibitor is provided comprising: analyzing the C3c level in a first urine sample from the subject; analyzing the C3c level in a subsequent urine sample from the subject, wherein the first urine sample is collected prior to the subsequent urine sample; comparing the C3c level in the first urine sample to the C3c level in the subsequent urine sample from the subject; and if the C3c level in the subsequent urine sample is equal to or greater than the C3c level in the first urine sample, then increasing the dose of the AP inhibitor administered to the subject. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method of monitoring the efficacy of an AP inhibitor treatment regimen in a human subject having an AP-associated nephropathy and receiving an AP inhibitor is provided comprising: analyzing the Ba level in a first urine sample from the subject; analyzing the Ba level in a subsequent urine sample from the subject, wherein the first urine sample is collected prior to the collection of the subsequent urine sample; comparing the Ba level in the first urine sample to the Ba level in the subsequent urine sample; and if the Ba level in the subsequent urine sample has insufficiently decreased compared to the Ba level in the first urine sample, then increasing the dose of the AP inhibitor administered to the subject. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method of monitoring the efficacy of an AP inhibitor treatment regimen in a human subject having C3GN and receiving an AP inhibitor is provided comprising: analyzing the Ba level in a first urine sample from the subject; analyzing the Ba level in a subsequent urine sample from the subject, wherein the first urine sample is collected prior to the collection of the subsequent urine sample; comparing the Ba level in the first urine sample to the Ba level in the subsequent urine sample; and if the Ba level in the subsequent urine sample has insufficiently decreased compared to the Ba level in the first urine sample, then increasing the dose of the AP inhibitor administered to the subject. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method of monitoring the efficacy of an AP inhibitor treatment regimen in a human subject having DDD and receiving an AP inhibitor is provided comprising: analyzing the Ba level in a first urine sample from the subject; analyzing the Ba level in a subsequent urine sample from the subject, wherein the first urine sample is collected prior to the collection of the subsequent urine sample; comparing the Ba level in the first urine sample to the Ba level in the subsequent urine sample; and if the Ba level in the subsequent urine sample has insufficiently decreased compared to the Ba level in the first urine sample, then increasing the dose of the AP inhibitor administered to the subject. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method of monitoring the efficacy of an AP inhibitor treatment regimen in a human subject having IC-MPGN and receiving an AP inhibitor is provided comprising: analyzing the Ba level in a first urine sample from the subject; analyzing the Ba level in a subsequent urine sample from the subject, wherein the first urine sample is collected prior to the collection of the subsequent urine sample; comparing the Ba level in the first urine sample to the Ba level in the subsequent urine sample; and if the Ba level in the subsequent urine sample has insufficiently decreased compared to the Ba level in the first urine sample, then increasing the dose of the AP inhibitor administered to the subject. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method of monitoring the efficacy of an AP inhibitor treatment regimen in a human subject having an AP-associated nephropathy and receiving an AP inhibitor is provided comprising: analyzing the sC5b-9 level in a first urine sample from the subject; analyzing the sC5b-9 level in a subsequent urine sample from the subject, wherein the first urine sample is collected prior to the collection of the subsequent urine sample; comparing the sC5b-9 level in the first urine sample to the sC5b-9 level in the subsequent urine sample; and if the sC5b-9 level in the subsequent urine sample has insufficiently decreased compared to the sC5b-9 level in the first urine sample, then increasing the dose of the AP inhibitor administered to the subject. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method of monitoring the efficacy of an AP inhibitor treatment regimen in a human subject having C3GN and receiving an AP inhibitor is provided comprising: analyzing the sC5b-9 level in a first urine sample from the subject; analyzing the sC5b-9 level in a subsequent urine sample from the subject, wherein the first urine sample is collected prior to the collection of the subsequent urine sample; comparing the sC5b-9 level in the first urine sample to the sC5b-9 level in the subsequent urine sample; and if the sC5b-9 level in the subsequent urine sample has insufficiently decreased compared to the sC5b-9 level in the first urine sample, then increasing the dose of the AP inhibitor administered to the subject. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method of monitoring the efficacy of an AP inhibitor treatment regimen in a human subject having DDD and receiving an AP inhibitor is provided comprising: analyzing the sC5b-9 level in a first urine sample from the subject; analyzing the sC5b-9 level in a subsequent urine sample from the subject, wherein the first urine sample is collected prior to the collection of the subsequent urine sample; comparing the sC5b-9 level in the first urine sample to the sC5b-9 level in the subsequent urine sample; and if the sC5b-9 level in the subsequent urine sample has insufficiently decreased compared to the sC5b-9 level in the first urine sample, then increasing the dose of the AP inhibitor administered to the subject. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method of monitoring the efficacy of an AP inhibitor treatment regimen in a human subject having IC-MPGN and receiving an AP inhibitor is provided comprising: analyzing the sC5b-9 level in a first urine sample from the subject; analyzing the sC5b-9 level in a subsequent urine sample from the subject, wherein the first urine sample is collected prior to the collection of the subsequent urine sample; comparing the sC5b-9 level in the first urine sample to the sC5b-9 level in the subsequent urine sample; and if the sC5b-9 level in the subsequent urine sample has insufficiently decreased compared to the sC5b-9 level in the first urine sample, then increasing the dose of the AP inhibitor administered to the subject. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method of monitoring the efficacy of an AP inhibitor treatment regimen in a human subject having an AP-associated nephropathy and receiving an AP inhibitor is provided comprising: analyzing the C3c level in a first urine sample from the subject; analyzing the C3c level in a subsequent urine sample from the subject, wherein the first urine sample is collected prior to the collection of the subsequent urine sample; comparing the C3c level in the first urine sample to the C3c level in the subsequent urine sample; and if the C3c level in the subsequent urine sample has insufficiently decreased compared to the C3c level in the first urine sample, then increasing the dose of the AP inhibitor administered to the subject. In some embodiments, the AP-inhibitor is a fD inhibitor. In some embodiments, the fD inhibitor is selected from Formula I, Formula II, and Compound 1-25, or a pharmaceutically acceptable salt thereof In some embodiments, the fD inhibitor is Compound 1, or a pharmaceutically acceptable salt thereof.

In some embodiments, a method of monitoring the efficacy of an AP inhibitor treatment regimen in a human subject having C3GN and receiving an AP inhibitor is provided comprising: analyzing the C3c level in a first urine sample from the subject; analyzing the C3c level in a subsequent urine sample from the subject, wherein the first urine sample is collected prior to the collection of the subsequent urine sample; comparing the C3c level in the first urine sample to the C3c level in the subsequent urine sample; and if the C3c level in the subsequent urine sample has insufficiently decreased compared to the C3c level in the first urine sample, then increasing the dose of the AP inhibitor administered to the subject. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method of monitoring the efficacy of an AP inhibitor treatment regimen in a human subject having DDD and receiving an AP inhibitor is provided comprising: analyzing the C3c level in a first urine sample from the subject; analyzing the C3c level in a subsequent urine sample from the subject, wherein the first urine sample is collected prior to the collection of the subsequent urine sample; comparing the C3c level in the first urine sample to the C3c level in the subsequent urine sample; and if the C3c level in the subsequent urine sample has insufficiently decreased compared to the C3c level in the first urine sample, then increasing the dose of the AP inhibitor administered to the subject. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In some embodiments, a method of monitoring the efficacy of an AP inhibitor treatment regimen in a human subject having IC-MPGN and receiving an AP inhibitor is provided comprising: analyzing the C3c level in a first urine sample from the subject; analyzing the C3c level in a subsequent urine sample from the subject, wherein the first urine sample is collected prior to the collection of the subsequent urine sample; comparing the C3c level in the first urine sample to the C3c level in the subsequent urine sample; and if the C3c level in the subsequent urine sample has insufficiently decreased compared to the C3c level in the first urine sample, then increasing the dose of the AP inhibitor administered to the subject. In some embodiments, the AP-inhibitor is a fD inhibitor. In some embodiments, the fD inhibitor is selected from Formula I, Formula II, and Compound 1-25, or a pharmaceutically acceptable salt thereof. In some embodiments, the fD inhibitor is Compound 1, or a pharmaceutically acceptable salt thereof.

In any of the above embodiments, the Ba level has decreased by less than about 10%. In any of the above embodiments, the Ba level has decreased by less than about 20%. In any of the above embodiments, the Ba level has decreased by less than about 30%. In any of the above embodiments, the Ba level has decreased by less than about 40%. In any of the above embodiments, the Ba level has decreased by less than about 50%. In any of the above embodiments, the Ba level has decreased by less than about 60%. In any of the above embodiments, the Ba level has decreased by less than about 70%. In any of the above embodiments, the Ba level has decreased by less than about 80%. In any of the above embodiments, the Ba level has decreased by less than about 90%.

In any of the above embodiments, the sC5b-9 level has decreased by less than about 10%. In any of the above embodiments, the sC5b-9 level has decreased by less than about 20%. In any of the above embodiments, the sC5b-9 level has decreased by less than about 30%. In any of the above embodiments, the sC5b-9 level has decreased by less than about 40%. In any of the above embodiments, the sC5b-9 level has decreased by less than about 50%. In any of the above embodiments, the sC5b-9 level has decreased by less than about 60%. In any of the above embodiments, the sC5b-9 level has decreased by less than about 70%. In any of the above embodiments, the sC5b-9 level has decreased by less than about 80%. In any of the above embodiments, the sC5b-9 level has decreased by less than about 90%.

In any of the above embodiments, the C3c level has decreased by less than about 10%. In any of the above embodiments, the C3c level has decreased by less than about 20%. In any of the above embodiments, the C3c level has decreased by less than about 30%. In any of the above embodiments, the C3c level has decreased by less than about 40%. In any of the above embodiments, the C3c level has decreased by less than about 50%. In any of the above embodiments, the C3c level has decreased by less than about 60%. In any of the above embodiments, the C3c level has decreased by less than about 70%. In any of the above embodiments, the C3c level has decreased by less than about 80%. In any of the above embodiments, the C3c level has decreased by less than about 90%.

In some embodiments, a method of treating a subject with an AP-pathway associated nephropathy is providing comprising administering to the subject an effective amount of Compound 1, or a pharmaceutically acceptable salt thereof; wherein the subject at the time of administration of Compound 1 has been or is currently receiving a therapeutic regimen comprising the administration of a C5 inhibitor; and wherein the Ba level in the subject's urine is greater than a range of Ba urine levels derived from individuals that do not have an AP-pathway associated nephropathy. In a particular embodiment, the C5 inhibitor is a monoclonal antibody to C5. In some embodiments, the C5 inhibitor is eculizumab. In some embodiments, the C5 inhibitor is ravulizumab-Cwvz. In some embodiments, the C5 inhibitor is a recombinant human minibody, for example Mubodina® (Adienne Pharma and Biotech). In some embodiments, the C5 inhibitor is coversin (Akari Therapeutics). In some embodiments, the C5 inhibitor is Tesidolumab/LFG316 (Novartis/Morphosys). In some embodiments, the C5 inhibitor is ARC-1905 (Iveric Bio). In some embodiments, the C5 inhibitor is RA101348 (Ra Pharmaceuticals). In some embodiments, the C5 inhibitor is RA101495 (Ra Pharmaceuticals). In some embodiments, the C5 inhibitor is SOBI002 (Swedish Orphan Biovitrum). In some embodiments, the C5 inhibitor is ARC1005 (Novo Nordisk). In some embodiments, the C5 inhibitor is a SOMAmer for C5 (SomaLogic). In some embodiments, the C5 inhibitor is SSL7. In some embodiments, the C5 inhibitor is MEDI7814 (MedImmune). In some embodiments, the C5 inhibitor is aurin tricarboxylic acid (Aurin Biotech). In another embodiment, the C5 inhibitor is an aurin tricarboxylic acid derivative (Aurin Biotech). In some embodiments, the C5 inhibitor is RG6107/SKY59 (Roche Pharmaceuticals). In another embodiment, the C5 inhibitor is ALXN5500 (Alexion Pharmaceuticals). In some embodiments, the C5 inhibitor is TT30 (Alexion Pharmaceuticals). In some embodiments, the C5 inhibitor is ABP959 (Amgen). In some embodiments, the C5 inhibitor is Anti-C5 siRNA (Alnylam Pharmaceuticals). In some embodiments, the C5 inhibitor is Erdigna (Adienne Pharma). In some embodiments, the C5 inhibitor is avacincaptad pegol/Zimura® (Iveric Bio). In some embodiments, the C5 inhibitor is SOBI005 (Swedish Orphan Biovitrum). In some embodiments, the C5 inhibitor is ISU305 (ISU ABXIS). In some embodiments, the C5 inhibitor is REGN3918 (Regeneron). In some embodiments, the subject has been on a C5 therapeutic regimen for at least 3-months prior to administration of Compound 1. In some embodiments, 100 mg of Compound 1 is administered three times a day. In some embodiments, 150 mg of Compound 1 is administered three tires a day. In some embodiments, 200 mg of Compound 1 is administered three times a day.

In some embodiments, a method of treating a subject with C3GN is providing comprising administering to the subject an effective amount of Compound 1, or a pharmaceutically acceptable salt thereof; wherein the subject at the time of administration of Compound 1 has been or is currently receiving a therapeutic regimen comprising the administration of a C5 inhibitor; and wherein the Ba level in the subject's urine is greater than a range of Ba urine levels derived from individuals that do not have C3GN. In a particular embodiment, the C5 inhibitor is a monoclonal antibody to C5. In some embodiments, the C5 inhibitor is eculizumab. In some embodiments, the C5 inhibitor is ravulizumab-Cwvz. In some embodiments, the C5 inhibitor is a recombinant human minibody, for example Mubodina® (Adienne Pharma and Biotech). In some embodiments, the C5 inhibitor is coversin (Akari Therapeutics). In some embodiments, the C5 inhibitor is Tesidolumab/LFG316 (Novartis/Morphosys). In some embodiments, the C5 inhibitor is ARC-1905 (Iveric Bio). In some embodiments, the C5 inhibitor is RA101348 (Ra Pharmaceuticals). In some embodiments, the C5 inhibitor is RA101495 (Ra Pharmaceuticals). In some embodiments, the C5 inhibitor is SOBI002 (Swedish Orphan Biovitrum). In some embodiments, the C5 inhibitor is ARC1005 (Novo Nordisk). In some embodiments, the C5 inhibitor is a SOMAmer for C5 (SomaLogic). In some embodiments, the C5 inhibitor is SSL7. In some embodiments, the C5 inhibitor is MEDI7814 (MedImmune). In some embodiments, the C5 inhibitor is aurin tricarboxylic acid (Aurin Biotech). In another embodiment, the C5 inhibitor is an aurin tricarboxylic acid derivative (Aurin Biotech). In some embodiments, the C5 inhibitor is RG6107/SKY59 (Roche Pharmaceuticals). In another embodiment, the C5 inhibitor is ALXN5500 (Alexion Pharmaceuticals). In some embodiments, the C5 inhibitor is TT30 (Alexion Pharmaceuticals). In some embodiments, the C5 inhibitor is ABP959 (Amgen). In some embodiments, the C5 inhibitor is Anti-05 siRNA (Alnylam Pharmaceuticals). In some embodiments, the C5 inhibitor is Erdigna (Adienne Pharma). In some embodiments, the C5 inhibitor is avacincaptad pegol/Zimura® (Iveric Bio). In some embodiments, the C5 inhibitor is SOBI005 (Swedish Orphan Biovitrum). In some embodiments, the C5 inhibitor is ISU305 (ISU ABXIS). In some embodiments, the C5 inhibitor is REGN3918 (Regeneron). In some embodiments, the subject has been on a C5 therapeutic regimen for at least 3-months prior to administration of Compound 1. In some embodiments, 100 mg of Compound 1 is administered three times a day. In some embodiments, 150 mg of Compound 1 is administered three times a day. In some embodiments, 200 mg of Compound 1 is administered three times a day.

In some embodiments, a method of treating a subject with DDD is providing comprising administering to the subject an effective amount of Compound 1, or a pharmaceutically acceptable salt thereof; wherein the subject at the time of administration of Compound 1 has been or is currently receiving a therapeutic regimen comprising the administration of a C5 inhibitor; and wherein the Ba level in the subject's urine is greater than a range of Ba urine levels derived from individuals that do not have DDD. In a particular embodiment, the C5 inhibitor is a monoclonal antibody to C5. In some embodiments, the C5 inhibitor is eculizumab. In some embodiments, the C5 inhibitor is ravulizumab-Cwvz. In some embodiments, the C5 inhibitor is a recombinant human minibody, for example Mubodina® (Adienne Pharma and Biotech). In some embodiments, the C5 inhibitor is coversin (Akari Therapeutics). In some embodiments, the C5 inhibitor is Tesidolumab/LFG316 (Novartis/Morphosys). In some embodiments, the C5 inhibitor is ARC-1905 (Iveric Bio). In some embodiments, the C5 inhibitor is RA101348 (Ra Pharmaceuticals). In some embodiments, the C5 inhibitor is RA101495 (Ra Pharmaceuticals). In some embodiments, the C5 inhibitor is SOBI002 (Swedish Orphan Biovitrum). In some embodiments, the C5 inhibitor is ARC1005 (Novo Nordisk). In some embodiments, the C5 inhibitor is a SOMAmer for C5 (SomaLogic). In some embodiments, the C5 inhibitor is SSL7. In some embodiments, the C5 inhibitor is MEDI7814 (MedImmune). In some embodiments, the C5 inhibitor is aurin tricarboxylic acid (Aurin Biotech). In another embodiment, the C5 inhibitor is an aurin tricarboxylic acid derivative (Aurin Biotech). In some embodiments, the C5 inhibitor is RG6107/SKY59 (Roche Pharmaceuticals). In another embodiment, the C5 inhibitor is ALXN5500 (Alexion Pharmaceuticals). In some embodiments, the C5 inhibitor is TT30 (Alexion Pharmaceuticals). In some embodiments, the C5 inhibitor is ABP959 (Amgen). In some embodiments, the C5 inhibitor is Anti-05 siRNA (Alnylam Pharmaceuticals). In some embodiments, the C5 inhibitor is Erdigna (Adienne Pharma). In some embodiments, the C5 inhibitor is avacincaptad pegol/Zimura® (Iveric Bio). In some embodiments, the C5 inhibitor is SOBI005 (Swedish Orphan Biovitrum). In some embodiments, the C5 inhibitor is ISU305 (ISU ABXIS). In some embodiments, the C5 inhibitor is REGN3918 (Regeneron). In some embodiments, the subject has been on a C5 therapeutic regimen for at least 3-months prior to administration of Compound 1. In some embodiments, 100 mg of Compound 1 is administered three times a day. In some embodiments, 150 mg of Compound 1 is administered three times a day. In some embodiments, 200 mg of Compound 1 is administered three times a day.

In some embodiments, a method of treating a subject with IC-MPGN is providing comprising administering to the subject an effective amount of Compound 1, or a pharmaceutically acceptable salt thereof; wherein the subject at the time of administration of Compound 1 has been or is currently receiving a therapeutic regimen comprising the administration of a C5 inhibitor; and wherein the Ba level in the subject's urine is greater than a range of Ba urine levels derived from individuals that do not have IC-MPGN. In a particular embodiment, the C5 inhibitor is a monoclonal antibody to C5. In some embodiments, the C5 inhibitor is eculizumab. In some embodiments, the C5 inhibitor is ravulizumab-Cwvz. In some embodiments, the C5 inhibitor is a recombinant human minibody, for example Mubodina® (Adienne Pharma and Biotech). In some embodiments, the C5 inhibitor is coversin (Akari Therapeutics). In some embodiments, the C5 inhibitor is Tesidolumab/LFG316 (Novartis/Morphosys). In some embodiments, the C5 inhibitor is ARC-1905 (Iveric Bio). In some embodiments, the C5 inhibitor is RA101348 (Ra Pharmaceuticals). In some embodiments, the C5 inhibitor is RA101495 (Ra Pharmaceuticals). In some embodiments, the C5 inhibitor is SOBI002 (Swedish Orphan Biovitrum). In some embodiments, the C5 inhibitor is ARC1005 (Novo Nordisk). In some embodiments, the C5 inhibitor is a SOMAmer for C5 (SomaLogic). In some embodiments, the C5 inhibitor is SSL7. In some embodiments, the C5 inhibitor is MEDI7814 (MedImmune). In some embodiments, the C5 inhibitor is aurin tricarboxylic acid (Aurin Biotech). In another embodiment, the C5 inhibitor is an aurin tricarboxylic acid derivative (Aurin Biotech). In some embodiments, the C5 inhibitor is RG6107/SKY59 (Roche Pharmaceuticals). In another embodiment, the C5 inhibitor is ALXN5500 (Alexion Pharmaceuticals). In some embodiments, the C5 inhibitor is TT30 (Alexion Pharmaceuticals). In some embodiments, the C5 inhibitor is ABP959 (Amgen). In some embodiments, the C5 inhibitor is Anti-05 siRNA (Alnylam Pharmaceuticals). In some embodiments, the C5 inhibitor is Erdigna (Adienne Pharma). In some embodiments, the C5 inhibitor is avacincaptad pegol/Zimura® (Iveric Bio). In some embodiments, the C5 inhibitor is SOBI005 (Swedish Orphan Biovitrum). In some embodiments, the C5 inhibitor is ISU305 (ISU ABXIS). In some embodiments, the C5 inhibitor is REGN3918 (Regeneron). In some embodiments, the subject has been on a C5 therapeutic regimen for at least 3-months prior to administration of Compound 1. In some embodiments, 100 mg of Compound 1 is administered three times a day. In some embodiments, 150 mg of Compound 1 is administered three times a day. In some embodiments, 200 mg of Compound 1 is administered three times a day.

In any of the above embodiments, the subject's Ba urine level is at least 2× higher than the upper limit of the normal Ba range. In any of the above embodiments, the subject's Ba urine level is at least 3× higher than the upper limit of the normal Ba range. In any of the above embodiments, the subject's Ba urine level is at least 4× higher than the upper limit of the normal Ba range. In any of the above embodiments, the subject's Ba urine level is at least 5× higher than the upper limit of the normal Ba range. In any of the above embodiments, the subject's Ba urine level is at least 6× higher than the upper limit of the normal Ba range. In any of the above embodiments, the subject's Ba urine level is at least 7× higher than the upper limit of the normal Ba range. In any of the above embodiments, the subject's Ba urine level is at least 8× higher than the upper limit of the normal Ba range. In any of the above embodiments, the subject's Ba urine level is at least 9× higher than the upper limit of the normal Ba range. In any of the above embodiments, the subject's Ba urine level is at least 10× higher than the upper limit of the normal Ba range. In any of the above embodiments, the subject's Ba urine level is at least 100× higher than the upper limit of the normal Ba range. In any of the above embodiments, the subject's Ba urine level is at least 200× higher than the upper limit of the normal Ba range. In any of the above embodiments, the subject's Ba urine level is at least 300× higher than the upper limit of the normal Ba range. In any of the above embodiments, the subject's Ba urine level is at least 400× higher than the upper limit of the normal Ba range. In any of the above embodiments, the subject's Ba urine level is at least 500× higher than the upper limit of the normal Ba range. In any of the above embodiments, the subject's Ba urine level is more than 500× higher than the upper limit of the normal Ba range.

In some embodiments, a method of treating a subject with an AP-pathway associated nephropathy is providing comprising administering to the subject an effective amount of Compound 1, or a pharmaceutically acceptable salt thereof; wherein the subject at the time of administration of Compound 1 has been or is currently receiving a therapeutic regimen comprising the administration of a C5 inhibitor; and wherein the Ba level in the subject's urine falls within a range of Ba urine levels derived from individuals that have an AP-pathway associated nephropathy. In a particular embodiment, the C5 inhibitor is a monoclonal antibody to C5. In some embodiments, the C5 inhibitor is eculizumab. In some embodiments, the C5 inhibitor is ravulizumab-Cwvz. In some embodiments, the C5 inhibitor is a recombinant human minibody, for example Mubodina® (Adienne Pharma and Biotech). In some embodiments, the C5 inhibitor is coversin (Akari Therapeutics). In some embodiments, the C5 inhibitor is Tesidolumab/LFG316 (Novartis/Morphosys). In some embodiments, the C5 inhibitor is ARC-1905 (Iveric Bio). In some embodiments, the C5 inhibitor is RA101348 (Ra Pharmaceuticals). In some embodiments, the C5 inhibitor is RA101495 (Ra Pharmaceuticals). In some embodiments, the C5 inhibitor is SOBI002 (Swedish Orphan Biovitrum). In some embodiments, the C5 inhibitor is ARC1005 (Novo Nordisk). In some embodiments, the C5 inhibitor is a SOMAmer for C5 (SomaLogic). In some embodiments, the C5 inhibitor is SSL7. In some embodiments, the C5 inhibitor is MEDI7814 (MedImmune). In some embodiments, the C5 inhibitor is aurin tricarboxylic acid (Aurin Biotech). In another embodiment, the C5 inhibitor is an aurin tricarboxylic acid derivative (Aurin Biotech). In some embodiments, the C5 inhibitor is RG6107/SKY59 (Roche Pharmaceuticals). In another embodiment, the C5 inhibitor is ALXN5500 (Alexion Pharmaceuticals). In some embodiments, the C5 inhibitor is TT30 (Alexion Pharmaceuticals). In some embodiments, the C5 inhibitor is ABP959 (Amgen). In some embodiments, the C5 inhibitor is Anti-C5 siRNA (Alnylam Pharmaceuticals). In some embodiments, the C5 inhibitor is Erdigna (Adienne Pharma). In some embodiments, the C5 inhibitor is avacincaptad pegol/Zimura® (Iveric Bio). In some embodiments, the C5 inhibitor is SOBI005 (Swedish Orphan Biovitrum). In some embodiments, the C5 inhibitor is ISU305 (ISU ABXIS). In some embodiments, the C5 inhibitor is REGN3918 (Regeneron). In some embodiments, the subject has been on a C5 therapeutic regimen for at least 3-months prior to administration of Compound 1. In some embodiments, 100 mg of Compound 1 is administered three times a day. In some embodiments, 150 mg of Compound 1 is administered three times a day. In some embodiments, 200 mg of Compound 1 is administered three times a day.

In some embodiments, a method of treating a subject with C3GN is providing comprising administering to the subject an effective amount of Compound 1, or a pharmaceutically acceptable salt thereof; wherein the subject at the time of administration of Compound 1 has been or is currently receiving a therapeutic regimen comprising the administration of a C5 inhibitor; and wherein the Ba level in the subject's urine falls within a range of Ba urine levels derived from individuals that have C3GN. In a particular embodiment, the C5 inhibitor is a monoclonal antibody to C5. In some embodiments, the C5 inhibitor is eculizumab. In some embodiments, the C5 inhibitor is ravulizumab-Cwvz. In some embodiments, the C5 inhibitor is a recombinant human minibody, for example Mubodina® (Adienne Pharma and Biotech). In some embodiments, the C5 inhibitor is coversin (Akari Therapeutics). In some embodiments, the C5 inhibitor is Tesidolumab/LFG316 (Novartis/Morphosys). In some embodiments, the C5 inhibitor is ARC-1905 (Iveric Bio). In some embodiments, the C5 inhibitor is RA101348 (Ra Pharmaceuticals). In some embodiments, the C5 inhibitor is RA101495 (Ra Pharmaceuticals). In some embodiments, the C5 inhibitor is SOBI002 (Swedish Orphan Biovitrum). In some embodiments, the C5 inhibitor is ARC1005 (Novo Nordisk). In some embodiments, the C5 inhibitor is a SOMAmer for C5 (SomaLogic). In some embodiments, the C5 inhibitor is SSL7. In some embodiments, the C5 inhibitor is MEDI7814 (MedImmune). In some embodiments, the C5 inhibitor is aurin tricarboxylic acid (Aurin Biotech). In another embodiment, the C5 inhibitor is an aurin tricarboxylic acid derivative (Aurin Biotech). In some embodiments, the C5 inhibitor is RG6107/SKY59 (Roche Pharmaceuticals). In another embodiment, the C5 inhibitor is ALXN5500 (Alexion Pharmaceuticals). In some embodiments, the C5 inhibitor is TT30 (Alexion Pharmaceuticals). In some embodiments, the C5 inhibitor is ABP959 (Amgen). In some embodiments, the C5 inhibitor is Anti-05 siRNA (Alnylam Pharmaceuticals). In some embodiments, the C5 inhibitor is Erdigna (Adienne Pharma). In some embodiments, the C5 inhibitor is avacincaptad pegol/Zimura® (Iveric Bio). In some embodiments, the C5 inhibitor is SOBI005 (Swedish Orphan Biovitrum). In some embodiments, the C5 inhibitor is ISU305 (ISU ABXIS). In some embodiments, the C5 inhibitor is REGN3918 (Regeneron). In some embodiments, the subject has been on a C5 therapeutic regimen for at least 3-months prior to administration of Compound 1. In some embodiments, 100 mg of Compound 1 is administered three times a day. In some embodiments, 150 mg of Compound 1 is administered three times a day. In some embodiments, 200 mg of Compound 1 is administered three times a day.

In some embodiments, a method of treating a subject with DDD is providing comprising administering to the subject an effective amount of Compound 1, or a pharmaceutically acceptable salt thereof; wherein the subject at the time of administration of Compound 1 has been or is currently receiving a therapeutic regimen comprising the administration of a C5 inhibitor; and wherein the Ba level in the subject's urine falls within a range of Ba urine levels derived from individuals that have DDD. In a particular embodiment, the C5 inhibitor is a monoclonal antibody to C5. In some embodiments, the C5 inhibitor is eculizumab. In some embodiments, the C5 inhibitor is ravulizumab-Cwvz. In some embodiments, the C5 inhibitor is a recombinant human minibody, for example Mubodina® (Adienne Pharma and Biotech). In some embodiments, the C5 inhibitor is coversin (Akari Therapeutics). In some embodiments, the C5 inhibitor is Tesidolumab/LFG316 (Novartis/Morphosys). In some embodiments, the C5 inhibitor is ARC-1905 (Iveric Bio). In some embodiments, the C5 inhibitor is RA101348 (Ra Pharmaceuticals). In some embodiments, the C5 inhibitor is RA101495 (Ra Pharmaceuticals). In some embodiments, the C5 inhibitor is SOBI002 (Swedish Orphan Biovitrum). In some embodiments, the C5 inhibitor is ARC1005 (Novo Nordisk). In some embodiments, the C5 inhibitor is a SOMAmer for C5 (SomaLogic). In some embodiments, the C5 inhibitor is SSL7. In some embodiments, the C5 inhibitor is MEDI7814 (MedImmune). In some embodiments, the C5 inhibitor is aurin tricarboxylic acid (Aurin Biotech). In another embodiment, the C5 inhibitor is an aurin tricarboxylic acid derivative (Aurin Biotech). In some embodiments, the C5 inhibitor is RG6107/SKY59 (Roche Pharmaceuticals). In another embodiment, the C5 inhibitor is ALXN5500 (Alexion Pharmaceuticals). In some embodiments, the C5 inhibitor is TT30 (Alexion Pharmaceuticals). In some embodiments, the C5 inhibitor is ABP959 (Amgen). In some embodiments, the C5 inhibitor is Anti-05 siRNA (Alnylam Pharmaceuticals). In some embodiments, the C5 inhibitor is Erdigna (Adienne Pharma). In some embodiments, the C5 inhibitor is avacincaptad pegol/Zimura® (Iveric Bio). In some embodiments, the C5 inhibitor is SOBI005 (Swedish Orphan Biovitrum). In some embodiments, the C5 inhibitor is ISU305 (ISU ABXIS). In some embodiments, the C5 inhibitor is REGN3918 (Regeneron). In some embodiments, the subject has been on a C5 therapeutic regimen for at least 3-months prior to administration of Compound 1. In some embodiments, 100 mg of Compound 1 is administered three times a day. In some embodiments, 150 mg of Compound 1 is administered three times a day. In some embodiments, 200 mg of Compound 1 is administered three times a day.

In some embodiments, a method of treating a subject with IC-MPGN is providing comprising administering to the subject an effective amount of Compound 1, or a pharmaceutically acceptable salt thereof; wherein the subject at the time of administration of Compound 1 has been or is currently receiving a therapeutic regimen comprising the administration of a C5 inhibitor; and wherein the Ba level in the subject's urine falls within a range of Ba urine levels derived from individuals that have IC-MPGN. In a particular embodiment, the C5 inhibitor is a monoclonal antibody to C5. In some embodiments, the C5 inhibitor is eculizumab. In some embodiments, the C5 inhibitor is ravulizumab-Cwvz. In some embodiments, the C5 inhibitor is a recombinant human minibody, for example Mubodina® (Adienne Pharma and Biotech). In some embodiments, the C5 inhibitor is coversin (Akari Therapeutics). In some embodiments, the C5 inhibitor is Tesidolumab/LFG316 (Novartis/Morphosys). In some embodiments, the C5 inhibitor is ARC-1905 (Iveric Bio). In some embodiments, the C5 inhibitor is RA101348 (Ra Pharmaceuticals). In some embodiments, the C5 inhibitor is RA101495 (Ra Pharmaceuticals). In some embodiments, the C5 inhibitor is SOBI002 (Swedish Orphan Biovitrum). In some embodiments, the C5 inhibitor is ARC1005 (Novo Nordisk). In some embodiments, the C5 inhibitor is a SOMAmer for C5 (SomaLogic). In some embodiments, the C5 inhibitor is SSL7. In some embodiments, the C5 inhibitor is MEDI7814 (MedImmune). In some embodiments, the C5 inhibitor is aurin tricarboxylic acid (Aurin Biotech). In another embodiment, the C5 inhibitor is an aurin tricarboxylic acid derivative (Aurin Biotech). In some embodiments, the C5 inhibitor is RG6107/SKY59 (Roche Pharmaceuticals). In another embodiment, the C5 inhibitor is ALXN5500 (Alexion Pharmaceuticals). In some embodiments, the C5 inhibitor is TT30 (Alexion Pharmaceuticals). In some embodiments, the C5 inhibitor is ABP959 (Amgen). In some embodiments, the C5 inhibitor is Anti-05 siRNA (Alnylam Pharmaceuticals). In some embodiments, the C5 inhibitor is Erdigna (Adienne Pharma). In some embodiments, the C5 inhibitor is avacincaptad pegol/Zimura® (Iveric Bio). In some embodiments, the C5 inhibitor is SOBI005 (Swedish Orphan Biovitrum). In some embodiments, the C5 inhibitor is ISU305 (ISU ABXIS). In some embodiments, the C5 inhibitor is REGN3918 (Regeneron). In some embodiments, the subject has been on a C5 therapeutic regimen for at least 3-months prior to administration of Compound 1. In some embodiments, 100 mg of Compound 1 is administered three times a day. In some embodiments, 150 mg of Compound 1 is administered three times a day. In some embodiments, 200 mg of Compound 1 is administered three times a day.

### Pharmaceutical Compositions and Dosage Forms

An AP inhibitor, for example a fB or fD inhibitor described herein, or its salt, isotopic analog, or prodrug can be administered in an effective amount to a host to treat any of the disorders described herein using any suitable approach which achieves the desired therapeutic result. The amount and timing of active compound administered will, of course, be dependent on the host being treated, the instructions of the supervising medical specialist, on the time course of the exposure, on the manner of administration, on the pharmacokinetic properties of the particular active compound, and on the judgment of the prescribing physician. Thus, because of host to host variability, the dosages given below are a guideline and the physician can titrate doses of the compound to achieve the treatment that the physician considers appropriate for the host. In considering the degree of treatment desired, the physician can balance a variety of factors such as age and weight of the host, presence of preexisting disease, as well as presence of other diseases.

The pharmaceutical composition may be formulated as any pharmaceutically useful form, e.g., as an aerosol, a cream, a gel, a pill, an injection or infusion solution, a capsule, a tablet, a syrup, a transdermal patch, a subcutaneous patch, a dry powder, an inhalation formulation, in a medical device, suppository, buccal, or sublingual formulation, parenteral formulation, or an ophthalmic solution. Some dosage forms, such as tablets and capsules, are subdivided into suitably sized unit doses containing appropriate quantities of the active components, e.g., an effective amount to achieve the desired purpose.

The therapeutically effective dosage of any active compound described herein will be determined by the health care practitioner depending on the condition, size and age of the subject as well as the route of delivery. In one non-limited embodiment, a dosage from about 0.1 to about 200 mg/kg has therapeutic efficacy, with all weights being calculated based upon the weight of the active compound, including the cases where a salt is employed. In some embodiments, the dosage is at about or greater than 0.1, 0.5, 1, 5, 10, 25, 50, 75, 100, 125, 150, 175, or 200 mg/kg. In some embodiments, the dosage may be the amount of compound needed to provide a serum concentration of the active compound of up to about 10 nM, 50 nM, 100 nM, 200 nM, 300 nM, 400 nM, 500 nM, 600 nM, 700 nM, 800 nM, 900 nM, 1 μM, 5 μM, 10 μM, 20 μM, 30 μM, or 40 μM.

In certain embodiments, the pharmaceutical composition is in a dosage form that contains from about 0.1 mg to about 2000 mg, from about 10 mg to about 1000 mg, from about 100 mg to about 800 mg, or from about 200 mg to about 600 mg of the active compound and optionally from about 0.1 mg to about 2000 mg, from about 10 mg to about 1000 mg, from about 100 mg to about 800 mg, or from about 200 mg to about 600 mg of an additional active agent in a unit dosage form. Examples include dosage forms with at least 5, 10, 15, 20, 25, 50, 100, 200, 250, 300, 400, 500, 600, 700, or 750 mg of active compound, or its salt. The pharmaceutical composition may also include a molar ratio of the active compound and an additional active agent, in a ratio that achieves the desired results.

Compounds disclosed herein or used as described herein may be administered orally, topically, parenterally, by inhalation or spray, sublingually, via implant, including ocular implant, transdermally, via buccal administration, rectally, as an ophthalmic solution, injection, including ocular injection, intravenous, intramuscular, inhalation, intra-aortal, intracranial, subdermal, intraperitoneal, subcutaneous, transnasal, sublingual, or rectal or by other means, in dosage unit formulations containing conventional pharmaceutically acceptable carriers. For ocular delivery, the compound can be administered, as desired, for example, via intravitreal, intrastromal, intracameral, sub-tenon, sub-retinal, retro-bulbar, peribulbar, suprachorodial, conjunctival, subconjunctival, episcleral, periocular, transscleral, retrobulbar, posterior juxtascleral, circumcorneal, or tear duct injections, or through a mucus, mucin, or a mucosal barrier, in an immediate or controlled release fashion or via an ocular device.

In accordance with the presently disclosed methods, an oral administration can be in any desired form such as a solid, gel or liquid, including a solution, suspension, or emulsion. In some embodiments, the compounds or salts are administered by inhalation, intravenously, or intramuscularly as a liposomal suspension. When administered through inhalation the active compound or salt may be in the form of a plurality of solid particles or droplets having any desired particle size, and for example, from about 0.01, 0.1 or 0.5 to about 5, 10, 20 or more microns, and optionally from about 1 to about 2 microns. Compounds as disclosed in the present invention have demonstrated good pharmacokinetic and pharmacodynamics properties, for instance when administered by the oral or intravenous routes.

The pharmaceutical formulations can comprise an active compound described herein or a pharmaceutically acceptable salt thereof, in any pharmaceutically acceptable carrier. If a solution is desired, water may sometimes be the carrier of choice for water-soluble compounds or salts. With respect to the water-soluble compounds or salts, an organic vehicle, such as glycerol, propylene glycol, polyethylene glycol, or mixtures thereof, can be suitable. In the latter instance, the organic vehicle can contain a substantial amount of water. The solution in either instance can then be sterilized in a suitable manner known to those in the art, and for illustration by filtration through a 0.22-micron filter. Subsequent to sterilization, the solution can be dispensed into appropriate receptacles, such as depyrogenated glass vials. The dispensing is optionally done by an aseptic method. Sterilized closures can then be placed on the vials and, if desired, the vial contents can be lyophilized.

Carriers include excipients and diluents and must be of sufficiently high purity and sufficiently low toxicity to render them suitable for administration to the subject being treated. The carrier can be inert or it can possess pharmaceutical benefits of its own. The amount of carrier employed in conjunction with the Compound 1s sufficient to provide a practical quantity of material for administration per unit dose of the compound.

Classes of carriers include, but are not limited to binders, buffering agents, coloring agents, diluents, disintegrants, emulsifiers, flavorants, glidents, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin; talc, and vegetable oils. Optional active agents may be included in a pharmaceutical composition, which do not substantially interfere with the activity of the compound of the present invention.

Additionally, auxiliary substances, such as wetting or emulsifying agents, biological buffering substances, surfactants, and the like, can be present in such vehicles. A biological buffer can be any solution which is pharmacologically acceptable and which provides the formulation with the desired pH, i.e., a pH in the physiologically acceptable range. Examples of buffer solutions include saline, phosphate buffered saline, Tris buffered saline, Hank's buffered saline, and the like.

Depending on the intended mode of administration, the pharmaceutical compositions can be in the form of solid, semi-solid or liquid dosage forms, such as, for example, tablets, suppositories, pills, capsules, powders, liquids, suspensions, creams, ointments, lotions or the like, preferably in unit dosage form suitable for single administration of a precise dosage. The compositions will include an effective amount of the selected drug in combination with a pharmaceutically acceptable carrier and, in addition, can include other pharmaceutical agents, adjuvants, diluents, buffers, and the like.

Thus, the compositions of the disclosure can be administered as pharmaceutical formulations including those suitable for oral (including buccal and sub-lingual), rectal, nasal, topical, pulmonary, vaginal or parenteral (including intramuscular, intra-arterial, intrathecal, subcutaneous and intravenous) administration or in a form suitable for administration by inhalation or insufflation. The preferred manner of administration is intravenous or oral using a convenient daily dosage regimen which can be adjusted according to the degree of affliction.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, and the like, an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered can also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, and the like. Actual methods of preparing such dosage forms are known, or will be apparent, to those skilled in this art; for example, see Remington's Pharmaceutical Sciences, referenced above.

In yet another embodiment is the use of permeation enhancer excipients including polymers such as: polycations (chitosan and its quaternary ammonium derivatives, poly-L-arginine, aminated gelatin); polyanions (N-carboxymethyl chitosan, poly-acrylic acid); and, thiolated polymers (carboxymethyl cellulose-cysteine, polycarbophil-cysteine, chitosan-thiobutylamidine, chitosan-thioglycolic acid, chitosan-glutathione conjugates).

For oral administration, the composition will generally take the form of a tablet, capsule, a soft gel capsule or can be an aqueous or nonaqueous solution, suspension or syrup. Tablets and capsules are preferred oral administration forms. Tablets and capsules for oral use can include one or more commonly used carriers such as lactose and corn starch. Lubricating agents, such as magnesium stearate, are also typically added. Typically, the compositions of the disclosure can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

When liquid suspensions are used, the active agent can be combined with any oral, non-toxic, pharmaceutically acceptable inert carrier such as ethanol, glycerol, water, and the like and with emulsifying and suspending agents. If desired, flavoring, coloring and/or sweetening agents can be added as well. Other optional components for incorporation into an oral formulation herein include, but are not limited to, preservatives, suspending agents, thickening agents, and the like.

Parenteral formulations can be prepared in conventional forms, either as liquid solutions or suspensions, solid forms suitable for solubilization or suspension in liquid prior to injection, or as emulsions. Preferably, sterile injectable suspensions are formulated according to techniques known in the art using suitable carriers, dispersing or wetting agents and suspending agents. The sterile injectable formulation can also be a sterile injectable solution or a suspension in a nontoxic parenterally acceptable diluent or solvent. Among the acceptable vehicles and solvents that can be employed are water, Ringer's solution and isotonic sodium chloride solution. In addition, sterile, fixed oils, fatty esters or polyols are conventionally employed as solvents or suspending media. In addition, parenteral administration can involve the use of a slow release or sustained release system such that a constant level of dosage is maintained.

Parenteral administration includes intraarticular, intravenous, intramuscular, intradermal, intraperitoneal, and subcutaneous routes, and include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Administration via certain parenteral routes can involve introducing the formulations of the disclosure into the body of a subject through a needle or a catheter, propelled by a sterile syringe or some other mechanical device such as a continuous infusion system. A formulation provided by the disclosure can be administered using a syringe, injector, pump, or any other device recognized in the art for parenteral administration.

In addition to the active compounds or their salts, the pharmaceutical formulations can contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the formulations can contain antimicrobial preservatives. Useful antimicrobial preservatives include methylparaben, propylparaben, and benzyl alcohol. An antimicrobial preservative is typically employed when the formulations is placed in a vial designed for multi-dose use. The pharmaceutical formulations described herein can be lyophilized using techniques well known in the art.

For oral administration, a pharmaceutical composition can take the form of a solution suspension, tablet, pill, capsule, powder, and the like. Tablets containing various excipients such as sodium citrate, calcium carbonate and calcium phosphate may be employed along with various disintegrants such as starch (e.g., potato or tapioca starch) and certain complex silicates, together with binding agents such as polyvinylpyrrolidone, sucrose, gelatin and acacia. Additionally, lubricating agents such as magnesium stearate, sodium lauryl sulfate, and talc are often very useful for tableting purposes. Solid compositions of a similar type may be employed as fillers in soft and hard-filled gelatin capsules. Materials in this connection also include lactose or milk sugar as well as high molecular weight polyethylene glycols. When aqueous suspensions and/or elixirs are desired for oral administration, the compounds of the presently disclosed host matter can be combined with various sweetening agents, flavoring agents, coloring agents, emulsifying agents and/or suspending agents, as well as such diluents as water, ethanol, propylene glycol, glycerin and various like combinations thereof.

In yet another embodiment of the host matter described herein, there are provided injectable, stable, sterile formulations comprising an active compound as described herein, or a salt thereof, in a unit dosage form in a sealed container. The compound or salt is provided in the form of a lyophilizate, which is capable of being reconstituted with a suitable pharmaceutically acceptable carrier to form liquid formulation suitable for injection thereof into a host. When the compound or salt is substantially water-insoluble, a sufficient amount of emulsifying agent, which is physiologically acceptable, can be employed in sufficient quantity to emulsify the compound or salt in an aqueous carrier. Particularly useful emulsifying agents include phosphatidyl cholines and lecithin.

Additional embodiments provided herein include liposomal formulations of the active compounds disclosed herein. The technology for forming liposomal suspensions is well known in the art. When the compound for administration is an aqueous-soluble salt, using conventional liposome technology, the same can be incorporated into lipid vesicles. In such an instance, due to the water solubility of the active compound, the active compound can be substantially entrained within the hydrophilic center or core of the liposomes. The lipid layer employed can be of any conventional composition and can either contain cholesterol or can be cholesterol-free. When the active compound of interest is water-insoluble, again employing conventional liposome formation technology, the salt can be substantially entrained within the hydrophobic lipid bilayer that forms the structure of the liposome. In either instance, the liposomes that are produced can be reduced in size, as through the use of standard sonication and homogenization techniques. The liposomal formulations comprising the active compounds disclosed herein can be lyophilized to produce a lyophilizate, which can be reconstituted with a pharmaceutically acceptable carrier, such as water, to regenerate a liposomal suspension.

Pharmaceutical formulations also are provided which are suitable for administration as an aerosol by inhalation. These formulations comprise a solution or suspension of a desired compound described herein or a salt thereof, or a plurality of solid particles of the compound or salt. The desired formulations can be placed in a small chamber and nebulized. Nebulization can be accomplished by compressed air or by ultrasonic energy to form a plurality of liquid droplets or solid particles comprising the compounds or salts. The liquid droplets or solid particles may for example have a particle size in the range of about 0.5 to about 10 microns, and optionally from about 0.5 to about 5 microns. In some embodiments, the solid particles provide for controlled release through the use of a degradable polymer. The solid particles can be obtained by processing the solid compound or a salt thereof, in any appropriate manner known in the art, such as by micronization. Optionally, the size of the solid particles or droplets can be from about 1 to about 2 microns. In this respect, commercial nebulizers are available to achieve this purpose. The compounds can be administered via an aerosol suspension of respirable particles in a manner set forth in U.S. Pat. No. 5,628,984, the disclosure of which is incorporated herein by reference in its entirety.

Pharmaceutical formulations also are provided which provide a controlled release of a compound described herein, including through the use of a degradable polymer, as known in the art.

### Representative Dosing Regimens of Compounds 1 to 25

In some embodiments, the dose of any of Compounds 1-25 administered to the subject is between about 25 mg and about 225 mg. In embodiment, the dose administered to the subject is between about 75 mg and about 125 mg. In some embodiments, the dosage administered is about 25 mg, about 50 mg, about 75 mg, about 100 mg, about 150 mg, about 200 mg, or about 250 mg. In some embodiments, any of Compounds 1-25 is administered in a single dose of about 50 mg. In some embodiments, any of Compounds 1-25 is administered in a single dose of about 75 mg. In some embodiments, any of Compounds 1-25 is administered in a single dose of about 100 mg. In some embodiments, any of Compounds 1-25 is administered in a single dose of about 150 mg. In some embodiments, any of Compounds 1-25 is administered in a single dose of about 200 mg. In some embodiments, any of Compounds 1-25 is administered in a single dose of about 225 mg.

In some embodiments, any of Compounds 1-25 is administered at least twice a day during treatment. In some embodiments, any of Compounds 1-25 is administered twice a day, with each dose spaced approximately 12 hours apart. In some embodiments, any of Compounds 1-25 is administered two times a day, with each dose spaced about 12 hours apart, for 28 days. In some embodiments, any of Compounds 1-25 is administered two times a day for 4 weeks, 5 weeks, 6 weeks, or 12 weeks. In some embodiments, any of Compounds 1-25 is administered two times a day for 3 months, 6 months, or 9 months. In some embodiments, 150 mg of any of Compounds 1-25 is administered twice a day, with each dose spaced approximately 12 hours apart. In some embodiments, 200 mg of any of Compounds 1-25 is administered twice a day, with each dose spaced approximately 12 hours apart. In some embodiments, 225 mg of any of Compounds 1-25 is administered twice a day, with each dose spaced approximately 12 hours apart.

In some embodiments, any of Compounds 1-25 is administered three times a day during treatment. In some embodiments, any of Compounds 1-25 is administered three times a day, with each dose spaced evenly apart. In some embodiments, any of Compounds 1-25 is administered three times a day, with each dose spaced about 8 hours apart. In some embodiments, any of Compounds 1-25 is administered three times a day, with each dose spaced about 8 hours apart, for a total of 28 days. In some embodiments, any of Compounds 1-25 is administered three times a day for 4 weeks, 5 weeks, 6 weeks, or 12 weeks. In some embodiments, any of Compounds 1-25 is administered three times a day for 3 months, 6 months, or 9 months. In some embodiments, any of Compounds 1-25 is administered on a continuous daily basis.

In some embodiments, any of Compounds 1-25 is administered at a dose of about 50 mg, 75 mg, 100 mg, 125 mg, 150 mg, 175 mg, or 200 mg, three times a day. In some embodiments, any of Compounds 1-25 is administered at a dose of about 75 mg, three times a day. In some embodiments, any of Compounds 1-25 is administered at a dose of about 75 mg, three times a day with each dose spaced approximately 8 hours apart. In some embodiments, any of Compounds 1-25 is administered at a dose of about 100 mg, three times a day. In some embodiments, any of Compounds 1-25 is administered at a dose of about 100 mg, three times a day, with each dose spaced approximately 8 hours apart. In some embodiments, any of Compounds 1-25 is administered at a dose of about 125 mg, three times a day. In some embodiments, any of Compounds 1-25 is administered at a dose of about 125 mg, three times a day, with each dose spaced approximately 8 hours apart. In some embodiments, any of Compounds 1-25 is administered at a dose of about 150 mg, three times a day. In some embodiments, any of Compounds 1-25 is administered at a dose of about 150 mg, three times a day, with each dose spaced approximately 8 hours apart. In some embodiments, any of Compounds 1-25 is administered at a dose of about 175 mg, three times a day. In some embodiments, any of Compounds 1-25 is administered at a dose of about 175 mg three, times a day, with each dose spaced approximately 8 hours apart. In some embodiments, any of Compounds 1-25 is administered at a dose of about 200 mg, three time a day. In some embodiments, any of Compounds 1-25 is administered at a dose of about 200 mg, three times a day, with each dose spaced approximately 8 hours apart.

### Albumin-to-Creatinine Ratio (ACR)

The presence of increased amounts of protein (albumin) in urine, or microalbuminuria, is a key indicator of kidney function and is routinely used to monitor the health of patients with kidney diseases. Albumin circulates throughout the body at high concentrations and is normally filtered by the kidney; however, in some disease states the kidney allows the protein to pass and be excreted into urine. The amount of albumin in a urine sample can be easily measured both quantitatively and qualitatively with a number of point-of-care techniques. Urine strips can be used to estimate both total protein and specifically albumin. Quantitative immunoassays are able to give accurate and specific measures of the protein. There are a number of tests available for urine creatinine estimation, the majority of which involve chemical or enzymatic reactions. An ACR measurement is reported in mg albumin/g creatinine and is widely accepted as an indicator of kidney function. Currently the National Kidney Foundation defines an ACR range from 30 to 300 mg/g as above normal, and multiple tests in this range over a three-month period suggest a problem.

Methods of measuring ACR are known to those skilled in the art and is generally measured in clinical reference labs using dipstick immunoassay kits. ACR results and a method to obtain the ACR results are shown in Example 2.

### Quantification of the Ba Fragment of Factor B in Human Urine, Plasma, and Serum.

The alternative complement pathway provides innate protection against microbial agents in the absence of specific antibody. The activation of this complement pathway can be triggered by a variety of substances including microbial polysaccharides or lipids, gram-negative bacterial lipopolysaccharides, and surface determinants present on some viruses, parasites, virally infected mammalian cells, and cancer cells. In autoimmune diseases, the alternative complement pathway may contribute directly to tissue damage.

A centrally important reaction that occurs during alternative pathway activation is the conversion of the 93 Kd molecular weight Factor B zymogen to an active proteolytic enzyme. This is accomplished in a two-step reaction. During the first reaction step the Factor B forms a magnesium-dependent complex with C3(H₂0) or C3b. The C3(H₂0), B complex is formed only in fluid-phase while the C3b, B complex can be formed either in fluid-phase or on a target surface. Factor B, which is present in the C3(H₂0), B or the C3b, B complex, is cleaved into the Ba (33 Kd) and Bb (60 Kd) fragments in the second reaction step by the alternative pathway enzyme, Factor D.

Although alternative pathway activation is thought to occur primarily in the absence of specific antibody, many situations arise in which alternative pathway activation can occur as the result of classical pathway activation. For example, immune complexes that are present in autoimmune disease patients can trigger classical complement pathway activation with resultant production of C3b fragments. As described above, these C3b molecules are capable of binding Factor B and initiating its cleavage into the Ba and Bb fragments. Thus, alternative pathway activation can occur in antibody-mediated autoimmune disease states and may contribute significantly to enhanced complement activation and concomitant tissue destruction.

By assessing Factor B cleavage products in test specimens, one can estimate the extent of alternative pathway utilization occurring at the time of sample collection in the disease state under investigation.

Concentrations of Ba levels can be measured by many methods known in the art including ELISA-based immunoassays, for example, as provided by the Quidel Microvue Ba EIA Kit A033. Ba results and a method to obtain Ba levels in serum and urine are shown in Examples 3 and 4. The reference range for “normal” levels of Ba in urine ranged from less than lower limit of quantitation (0.033 ng/ml) to 3.32 ng/ml. A Ba urine levels greater than a normal reference range may be indicative of an AP-associated nephropathy, for example C3G or IC-MPGN.

### Quantification of the Bb Fragment of Factor B in Human Urine, Plasma, and Serum.

The alternative complement pathway provides innate protection against microbial agents in the absence of specific antibody. The activation of this complement pathway can be triggered by a variety of substances including microbial polysaccharides or lipids, gram-negative bacterial lipopolysaccharides, and surface determinants present on some viruses, parasites, virally infected mammalian cells, and cancer cells. In autoimmune diseases, the alternative complement pathway may contribute directly to tissue damage.

A centrally important reaction that occurs during alternative pathway activation is the conversion of the 93 Kd molecular weight Factor B zymogen to an active proteolytic enzyme. This is accomplished in a two-step reaction. During the first reaction step the Factor B forms a magnesium-dependent complex with C3(H₂0) or C3b. The C3(H₂0), B complex is formed only in fluid-phase while the C3b, B complex can be formed either in fluid-phase or on a target surface. Factor B, which is present in the C3(H₂0), B or the C3b, B complex, is cleaved into the Ba (33 Kd) and Bb (60 Kd) fragments in the second reaction step by the alternative pathway enzyme, Factor D. The resulting C3b, Bb bimolecular complex is the C3 convertase enzyme of the alternative pathway. The Bb subunit is the catalytically active site of the complex that is capable of cleaving C3 to C3a and C3b fragments. The additional C3b fragments produced in this manner may form the C3b, Bb, C3b trimolecular complex that is the C5 convertase enzyme of the alternative pathway. This C5 convertase is capable of cleaving C5 to C5a and C5b fragments.

The C3 and C5 convertases of the alternative pathway can be stabilized by Factor P (also called Properdin), a component of the alternative pathway normally present in human plasma or serum, 1-4 or by C3 nephritic factor, an autoantibody produced in some patients experiencing extensive alternative pathway activation. The C3 and C5 convertases of the alternative pathway can be dissociated, and thereby inactivated, by spontaneous decay dissociation, or by the binding of Factor H or Complement Receptor 1 (CR1). The Bb fragment that is dissociated from either convertase retains some biological activities, e.g., retention of functional hemolytic activity, the ability to induce macrophage-spreading, and plasminogen activation.

Although alternative pathway activation is thought to occur primarily in the absence of specific antibody, many situations arise in which alternative pathway activation can occur as the result of classical pathway activation. For example, immune complexes that are present in autoimmune disease patients can trigger classical complement pathway activation with resultant production of C3b fragments. As described above, these C3b molecules are capable of binding Factor B and initiating its cleavage into the Ba and Bb fragments. Thus, alternative pathway activation can occur in antibody-mediated autoimmune disease states and may contribute significantly to enhanced complement activation and concomitant tissue destruction.

Concentrations of Bb levels can be measured by many methods known in the art including ELISA-based immunoassays. Bb results and a method to obtain Bb levels in plasma are shown in Example 3. The reference range for “normal” levels of Bb in plasma was 0.49-1.42 μg/mL. Bb plasma levels greater than the reference normal range may be indicative of an AP-associated nephropathy.

Quantification of the sC5b-9 Complex in Human Urine

The Terminal Complement Complex (TCC, sC5b-9) is generated by the assembly of C5 through C9 as a consequence of activation of the complement system by either the classical, lectin or alternative pathway. The membrane attack complex (MAC), a form of TCC, is a stable complex that mediates the irreversible target cell membrane damage associated with complement activation. Complexes formed in the absence of a target membrane bind to naturally occurring regulatory serum proteins, e.g. the S protein, at the C5b-7 stage of assembly forming soluble, non-lytic TCC. For purposes of this document, we refer to all forms of stable Terminal Complement Complex interchangeably as TCC and sC5b-9, recognizing that other complement regulatory proteins, like Clusterin, also form these stable complexes and are detectable in sC5b-9 assays.

Concentrations of sC5b-9 can be measured by many methods known in the art including ELISA-based immunoassays thereby giving an indication of the status of the terminal complement pathway in the specimen. An example of an ELISA-based immunoassay includes the A020 Micro Vue sC5b-9 Enzyme Immunoassay for the quantitation of sC5b-9 in human urine, plasma, and serum. sC5b-9 results and a method to obtain sC5b-9 levels in urine are shown in Examples 3 and 4. The reference range for “normal” levels of sC5b-9 levels in urine was below the lower limit of quantitation (8.8 ng/ml). A sC5b-9 urine level greater than 3.09 ng/mL may be indicative of an AP-associated nephropathy, for example, C3G or IC-MPGN.

### Quantification of the C3c Fragment in Human Urine

The complement system mediates a number of essential biological functions that participate in host defense against infection, initiation of the inflammatory reaction, processing and clearance of immune complexes and regulation of the immune response. There are three pathways of complement activation: the classical pathway is initiated by immune complexes, the lectin pathway by surface bound mannan binding lectin and the alternative pathway by all the surfaces that are not specifically protected against it. Each complement pathway generates a C3 convertase, a serine protease that cleaves the central complement protein C3, and generates the major cleavage fragment C3b. C3b is an opsonin and part of one of the main convertases that drives the complement cascade. In the presence of complement regulatory molecules C3b may be further degraded sequentially to iC3b, C3c, C3dg and C3d. The disadvantage of most complement biomarkers is their short half-life, making reliable sample collection and measurements difficult. Unlike other C3 fragments, C3c does not bind to other structures like pathogens, cell surface (receptors) and other plasma proteins. Therefore, C3c is a stable complement biomarker which will appear in the fluid phase only, without interference of other C3 based products. The measurement of C3c provides evidence of (uncontrolled) complement activation and can be used as an indicator of an inflammatory state.

Methods to measure C3c levels are known in the art and include C3c ELISA assays, based on an antibody highly specific for an epitope exclusively on C3c, for example as described in the Human C3c Elisa Kit from Hycult Biotech HK368. C3c results and a method to obtain C3c levels in urine are shown in Example 6. The reference range for “normal” levels of C3 in serum was determined to be <LLOQ (1.6 ng/ml). A C3c urine level greater than the normal reference range may be indicative of an AP-associated nephropathy, for example C3G or IC-MPGN.

### Quantification of Factor D Levels in Serum

The alternative pathway of complement represents an important humoral component of natural defense against microbial attack. The interaction of the proteins C3, factor B, and factor D results in the formation of the alternative C3- and C5-convertases, i.e. C3bBb and C3bBbC3b(n). These multicomponent enzymes assemble on the surface of alternative pathway of complement activators and are stabilized by properdin (P). The participation of the alternative pathway of complement has been implicated in the pathogenesis of a wide variety of human diseases.

Factor D, a 24 kD serine protease of the alternative complement pathway, is synthesized as a precursor single-chain molecule. Factor D is unique among serine proteases because it requires neither enzymatic cleavage for expression of proteolytic activity nor activation by a serpin for its control. Factor D is highly specific and cleaves factor B bound to C3b, generating the C3bBb enzyme. Factor D is the rate-linking C3 convertase enzyme of the alternative pathway. Furthermore, factor D plays a role in fatty tissue distinct from its role as a complement protein. Normal values in human EDTA plasma are 1.05 (±0.27) μg/ml. In healthy individuals factor D is rapidly eliminated via the kidney and neither modified extrarenal catabolism nor changes in synthesis contribute to elevated factor D levels observed in patients with renal failure. The levels in patients with chronic renal failure (CRF) increase up to 4.35 (±3.03) μg/ml and in end-stage renal disease (ERDS) up to 12.1 (±3.53) μg/ml. Measurable quantities of factor D were detected in urine of 85% of healthy individuals (0.62±0.33 ng/ml). The urinary concentrations of factor D in patients with tubular proteinuria are elevated to 230 (±313) ng/ml.

Methods to measure Factor D levels are known in the art and include Factor D ELISA assays, based on an antibody highly specific for human Factor D. Factor D results and a method to obtain Factor D levels in serum are shown in Example 3. The reference range for “normal” levels of Factor D in serum is 1.73-3.24 μg/mL. As shown in Example 3, Factor D levels in serum are within the normal range despite a confirmed diagnosis of an AP-associated nephropathy, which suggests that serum levels of Factor D are not predictive of an AP-associated nephropathy.

### Quantification of C3 Levels in Serum

Complement component 3 (C3) plays a central role in all three complement activation pathways. The C3 precursor contains 1,663 amino acids and has a molecular weight of about 180 kDa. Human C3 has 77% identity to mouse C3 at the amino acid level. C3 is cleaved by C3 convertase into two activated fragments C3a and C3b. The anaphylatoxin C3a is a vasoactive peptide and a mediator of local inflammatory process. The C3b in complex with receptor can bind covalently to pathogen surfaces to promote phagocytosis. Acquired C3 deficiency is associated with severe recurrent meningococci and pneumococci infections. Plasma C3 and C3a levels are elevated in cryptogenic and large-vessel disease subtypes of ischemic stroke.

Methods to measure C3 levels are known in the art and include C3 ELISA assays, based on an antibody highly specific for human C3. C3 results and a method to obtain C3 levels in serum are shown in Example 3. The reference range for “normal” levels of C3 in serum is 0.78-1.82 mg/mL. As shown in Example 3, C3 levels in serum are within the normal range despite a confirmed diagnosis of an AP-associated nephropathy, which suggests that serum levels of C3 are not predictive of an AP-associated nephropathy.

### Quantification of C4 Levels in Serum

Complement component 4 (C4) plays a key role in the activation of the classical complement pathway. C4 is synthesized as a single-chain precursor molecule (200 kDa) but processed to the three-chain disulphide-linked structure with alpha (93 kDa), beta (78 kDa) and gamma (33 kDa) chains prior to secretion. After activation by C1s, C4 is processed to C4a and C4b. C4a anaphylatoxin is a mediator of local inflammation and induces smooth muscle contraction. C4b, the major activation product, is an essential subunit of the C3 and C5 convertases of the classical complement pathway. C4 deficiency is associated with systemic lupus erythematosus. The C4b degradation product C4d is a marker for humoral rejection in allografts.

Methods to measure C4 levels are known in the art and include C4 ELISA assays, based on an antibody highly specific for human C4. C4 results and a method to obtain C4 levels in serum are shown in Example 4. The reference range for “normal” levels of C4 in serum is 10-40 mg/mL. As shown in Example 3, C4 levels in serum are within the normal range despite a confirmed diagnosis of an AP-associated nephropathy, which suggests that serum levels of C4 are not predictive of an AP-associated nephropathy.

### EXPERIMENTAL EXAMPLES

### Example 1. A Phase 2a Proof-of-Mechanism, Open-Label Study to Determine the Effect of Compound 1 on C3 Levels in Patients with Low C3 Levels Due to Either C3 Glomerulopathy (C3G) or Immune-Complex Membranoproliferative Glomerulonephritis (IC-MPGN) (ClinicalTrials.Gov Identifier: NCT03124368)

The primary objective of this study is to determine whether Compound 1 can increase blood C3 levels in participants with low C3 levels due to either C3 Glomerulopathy (C3G) or Immune-Complex Membranoproliferative Glomerulonephritis (IC-MPGN).

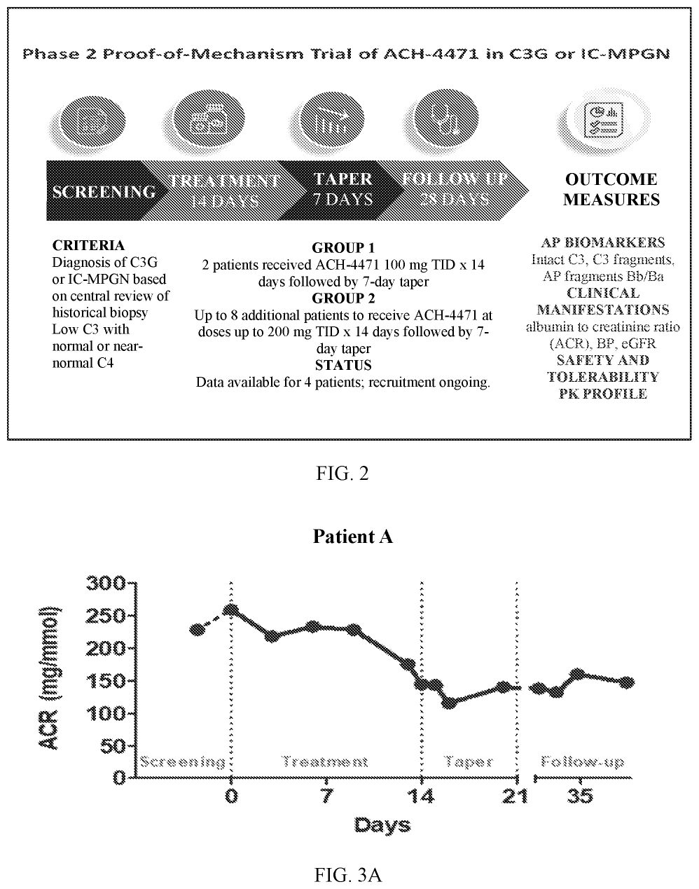

10 patients with either C3 Glomerulopathy (C3G) or Immune-Complex Membranoproliferative Glomerulonephritis (IC-MPGN) are to be enrolled across two dose groups. Group 1 will contain 2 patients who will receive Compound 1 dosed at 100 mg TID×14 days followed by a 7-day taper. Group 2 will contain up to 8 additional patients who will receive Compound 1 dosed at 200 mg TID×14 days followed by a 7-day taper. Inclusion criteria include a clinical diagnosis of C3G (C3 glomerulonephritis [C3GN] or dense deposit disease [DDD], the 2 types of C3G) or idiopathic immune-complex membranoproliferative glomerulonephritis (IC-MPGN) by renal biopsy for at least 3 months prior to dosing, with the pathologic diagnosis verified by review of the renal biopsy by the study central pathologist. Patients must have low C3 levels (<50% of the lower limit of normal) and normal or near-normal C4 levels (>90% of the lower limit of normal). Group 1 has enrolled 2 out of 2 planned patients and Group 2 has enrolled 4 out of 8 planned patients. An overview of the clinical trial is provided in FIG. 2.

Table 1 below provides key demographic information for the 6 subjects enrolled in both Groups 1 and 2.

TABLE 1

| Group | Patient | Renal Biopsy Diagnosis |
|---|---|---|
| 1 | A | C3GN |
| 1 | B | IC-MPGN |
| 2 | C | C3GN |
| 2 | D | C3GN |
| 2 | E | C3 glomerulopathy with a |
| | | mebranoproliferative pattern |
| 2 | F | C3GN |

### Example 2. Reduction in ACR with 14-Day Compound 1 Treatment

Urine samples were collected from patients A, B, C, D, E, and F at protocol-specified timepoints prior to, during, and after dosing with Compound 1. Albumin and creatinine levels were measured in urine samples by a central reference lab using a urine dipstick method. Urine from non-study healthy volunteers served as control samples. An automatic urinary chemical analyzer was used with a dye for the detection of albumin at >8 mg/dL. Also present on the dipstick was a pad for detection of creatinine that uses the peroxidase activity of the copper-creatinine complex. The ACR ratio was automatically calculated by the analyzer based on the light absorbent readings of the albumin and creatinine test pads.

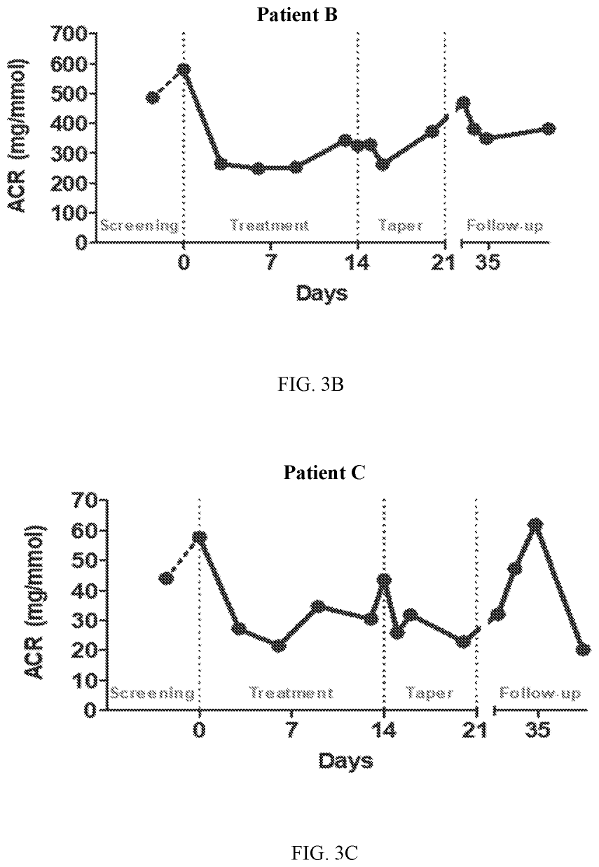

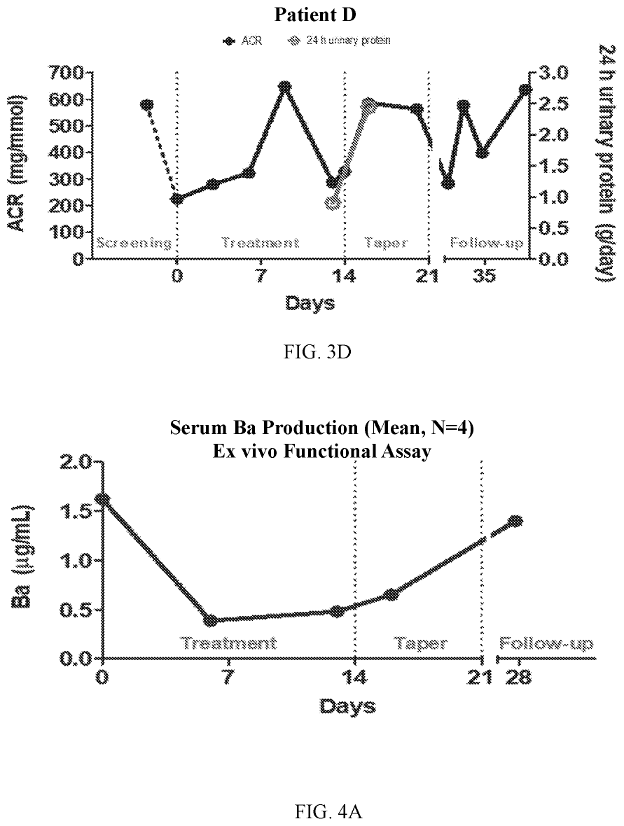

Patients A, B, C, E, and F showed approximately 50% reductions in ACR during a two-week treatment with Compound 1 (See FIGS. 3A-3C regarding Patients A, B, and C). Patient D showed highly variable ACR values; accordingly, 24-hour urinary proteins were collected on days 14 and 17. The body weight of the patient is only 39 kg and the usual variable urinary creatinine levels were observed (See FIG. 3D). The results for all six patients are shown below in Table 2.

TABLE 2

| | ACR reported mg/mmol | | | | | | | | |
|---|---|---|---|---|---|---|---|---|---|
| | A | C | B | D | E | F | MEAN | GEOMEAN | N |
| Screen 1 | ND | 128 | ND | 160 | ND | ND | | | |
| Screen | 228 | 44 | 486 | 579 | ND | 308 | | | |
| Day 1/0 Hour | 259 | 58 | 580 | 224 | 424 | 293 | 306.4 | 249.6 | 6 |
| Day 4 | 218 | 27 | 264 | 279 | 418 | 152 | 226.3 | 174.0 | 6 |
| Day 7/0 Hour | 233 | 22 | 249 | 322 | 533 | 94 | 242.1 | 164.9 | 6 |
| Day 10 | 228 | 35 | 253 | 648 | 625 | ND | | | |
| Day 14 | 175 | 30 | 343 | 286 | 264 | 184 | 213.8 | 171.4 | 6 |
| Day 15 Taper | 144 | 44 | 325 | 328 | 327 | 720 | 314.6 | 232.4 | 6 |
| Day 16 Taper | 143 | 26 | 329 | ND | 277 | 235 | | | |
| Day 17 Taper | 115 | 32 | 263 | 586 | 475 | 475 | 324.2 | 224.2 | 6 |
| Day 21 Taper | 140 | 23 | 373 | 564 | 257 | 348 | 284.0 | 197.9 | 6 |
| Follow-Up Day 24 | 138 | 32 | 469 | 282 | 303 | 279 | 250.6 | 191.6 | 6 |
| Follow-Up Day 28 | 132 | 47 | 381 | 577 | 307 | 253 | 282.6 | 217.5 | 6 |
| Follow-Up Day 35 | 160 | ND | ND | 398 | ND | 168 | | | |
| Follow-Up Day 49 | 147 | 20 | 140 | 636 | 327 | 564 | 305.6 | 191.1 | 6 |

### Example 3. Systemic Complement Proteins: Trends with 14-Day Compound 1 Treatment

Systemic complement proteins were measured in serum and plasma including serum Ba, plasma Bb, serum C3, serum Factor D, serum C4, and % Fragment C3. Serum and plasma samples were collected from patients A, B, C, D, E, and F at protocol-specified timepoints prior to, during, and after dosing with Compound 1.

### Ba Production in Serum

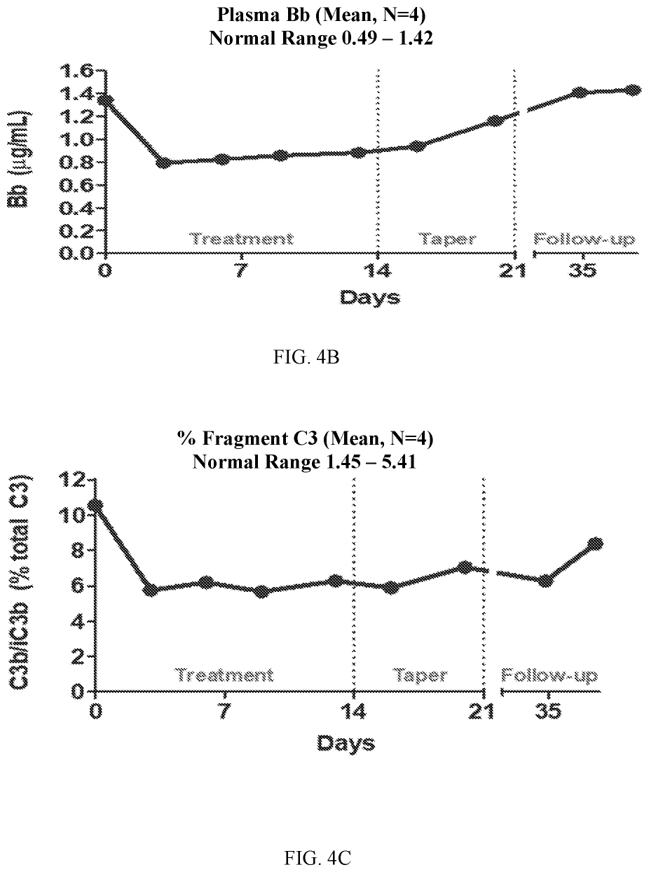

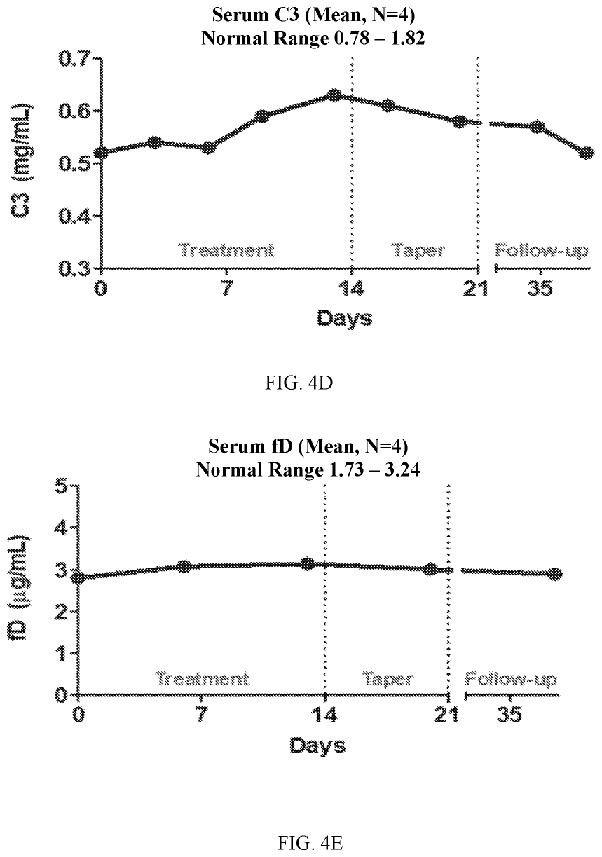

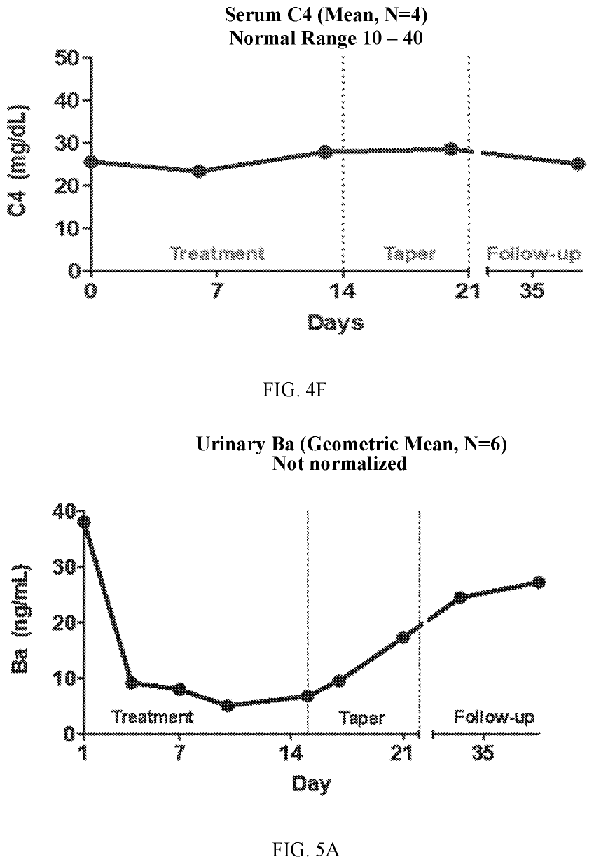

To measure serum Ba production, the serum samples measurements were performed ex vivo after incubation of serum samples in the presence of exogenous C3 at 37° C. for 30 mins. Patient samples were then tested utilizing the MicroVue Ba Plus Enzyme Immunoassay for the quantitation of Ba in serum. This is a three-step procedure that utilizes a micro-assay plate coated with a mouse monoclonal antibody that binds specifically to human Ba, a horseradish peroxidase (HRP)-conjugated polyclonal anti-human Factor B, and a chromogenic substrate. In step 1, standards, controls, and test specimens were added to micro-assay wells pre-coated with a specific anti-Ba monoclonal antibody. Ba, but not Factor B or other complement activation products, present in the Standards, Controls, or specimens bind to the immobilized anti-Ba monoclonal antibody. After incubation, a wash cycle removed unbound material. In step 2, horseradish peroxidase (HRP)-conjugated polyclonal anti-Factor B antibody was added to each test well. The enzyme conjugated anti-Factor B binds to Ba captured in the micro-assay wells. After incubation, a wash cycle removed unbound, excess conjugate. In step 3, a chromogenic enzyme substrate was added to each micro-assay well. The bound HRP-conjugate reacted with the substrate, forming a blue color. After incubation the enzyme reaction was stopped chemically, the color changed to yellow, and the color intensity was measured spectrophotometrically at 450 nm. The color intensity of the reaction mixture is proportional to the concentration of Ba present in the test specimens, Standards, and Controls. The results of patients A, B, C, and D were pooled and are shown in FIG. 4A. Patients E and F (not shown) show similar results. Ex vivo serum Ba production, a functional assay for AP C3 convertase formation, was inhibited with Compound 1 treatment.

### Bb Levels in Plasma

To measure plasma Bb production, patient samples were tested utilizing the MicroVue Bb Enzyme Immunoassay for the quantitation of Bb in plasma. This is a three-step procedure that utilizes a micro-assay plate coated with a mouse monoclonal antibody that binds specifically to human Bb, a horseradish peroxidase (HRP)-conjugated polyclonal anti-human Factor B, and a chromogenic substrate. In step 1, standards, controls, and test specimens were added to micro-assay wells pre-coated with a specific anti-Bb monoclonal antibody. Bb, but not Factor B or other complement activation products, present in the Standards, Controls, or specimens bind to the immobilized anti-Bb monoclonal antibody. After incubation, a wash cycle removed unbound material. In step 2, horseradish peroxidase (HRP)-conjugated polyclonal anti-Factor B antibody was added to each test well. The enzyme conjugated anti-Factor B binds to Bb captured in the micro-assay wells. After incubation, a wash cycle removed unbound, excess conjugate. In step 3, a chromogenic enzyme substrate was added to each micro-assay well. The bound HRP-conjugate reacted with the substrate, forming a blue color. After incubation the enzyme reaction was stopped chemically, the color changed to yellow, and the color intensity was measured spectrophotometrically at 450 nm. The color intensity of the reaction mixture is proportional to the concentration of Bb present in the test specimens, Standards, and Controls. The results for patients A, B, C, and D were pooled and are shown in FIG. 4B. Patients E and F (not shown) show similar results. Plasma Bb was reduced with Compound 1 treatment.

### C3 Levels in Serum

Serum and plasma samples were collected from patients A, B, C, D, E and F at protocol-specified timepoints prior to, during, and after dosing with Compound 1. C3 levels in serum were measured using a quantitative sandwich enzyme immunoassay technique. A monoclonal antibody specific for human C3 was pre-coated onto a micro-assay plate. Standards and samples were pipetted into the wells and any C3 present was bound by the immobilized antibody. After washing away any unbound substances, an enzyme-linked polyclonal antibody specific for human C3 was added to the wells. Following a wash to remove any unbound antibody-enzyme reagent, a substrate solution was added to the wells and color develops in proportion to the amount of C3 bound in the initial step. The color development was stopped and the intensity of the color was measured. Serum C3 was increased with Compound 1 treatment (FIG. 4D, showing pooled patients A, B, C, and D) accompanied with reduced % fragment C3 (FIG. 4C, showing pooled patients A, B, C, and D), indicating lower C3 consumption in vivo. Patients E and F show similar results.

### Factor D Levels in Plasma

Serum and plasma samples were collected from patients A, B, C, D, E, and F at protocol-specified timepoints prior to, during, and after dosing with Compound 1. Factor D levels in serum were measured using a quantitative sandwich enzyme immunoassay technique. A monoclonal antibody specific for human Complement Factor D was pre-coated onto a micro-assay plate. Standards and samples were pipetted into the wells and any Complement Factor D present was bound by the immobilized antibody. After washing away any unbound substances, an enzyme-linked polyclonal antibody specific for human Complement Factor D was added to the wells. Following a wash to remove any unbound antibody-enzyme reagent, a substrate solution was added to the wells and color develops in proportion to the amount of Complement Factor D bound in the initial step. The color development was stopped and the intensity of the color was measured spectrophotometrically at 450 nm. Serum Factor D remained unchanged during Compound 1 treatment as shown in FIG. 4E showing pooled patients A, B, C, and D. Patients E and F show similar results.

### C4 Levels in Serum

Serum and plasma samples were collected from patients A, B, C, and D at protocol-specified timepoints prior to, during, and after dosing with Compound 1. C4 levels in serum were measured using a quantitative sandwich enzyme immunoassay technique. A monoclonal antibody specific for human C4 was pre-coated onto a micro-assay plate. Standards and samples were pipetted into the wells and any C4 present was bound by the immobilized antibody. After washing away any unbound substances, an enzyme-linked polyclonal antibody specific for human C4 was added to the wells. Following a wash to remove any unbound antibody-enzyme reagent, a substrate solution was added to the wells and color develops in proportion to the amount of C4 bound in the initial step. The color development was stopped and the intensity of the color was measured spectrophotometrically at 450 nm. Serum C4 remained unchanged during Compound 1 treatment as shown in FIG. 4F, showing pooled patients A, B, C, and D. Patients E and F show similar results.

### Example 4. Urinary Complement Biomarkers: Trends with 14-Day Compound 1 Treatment

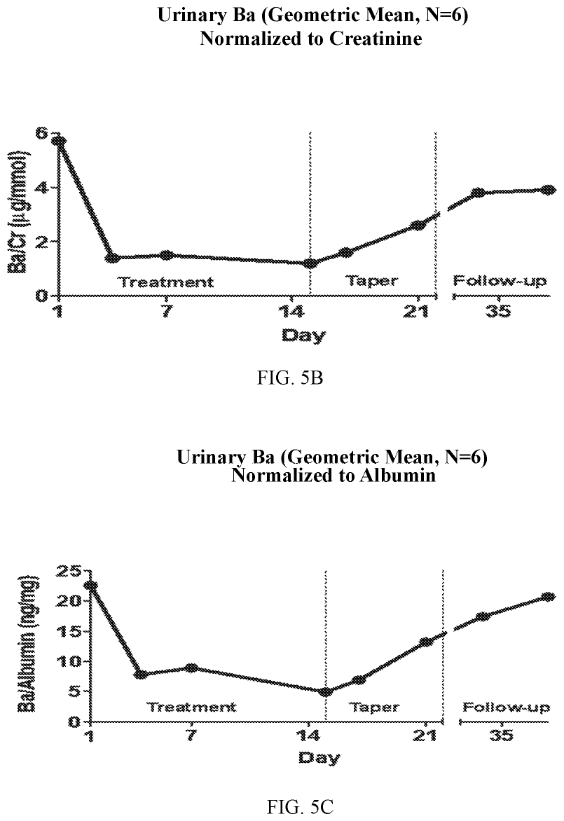

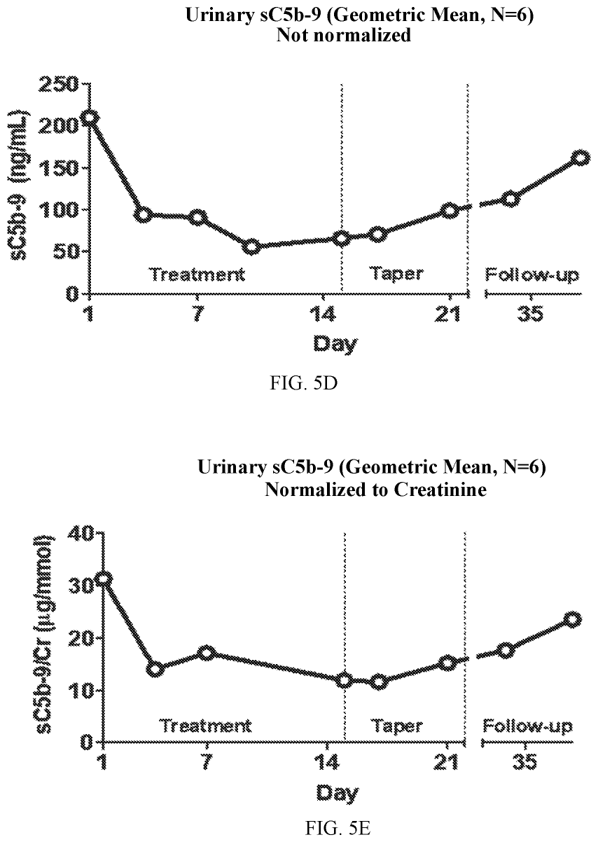

Urine samples were collected from patients A, B, C, D E, and F at protocol-specified timepoints prior to, during, and after dosing with Compound 1. To measure urinary Ba concentration, patient urine samples were tested utilizing the MicroVue Ba Enzyme Immunoassay for the quantitation of Ba in urine. This is a three-step procedure that utilizes a micro-assay plate coated with a mouse monoclonal antibody that binds specifically to human Ba, a horseradish peroxidase (HRP)-conjugated polyclonal anti-human Factor B, and a chromogenic substrate. In step 1, standards, controls, and test specimens were added to micro-assay wells pre-coated with a specific anti-Ba monoclonal antibody. Ba, but not Factor B or other complement activation products, present in the Standards, Controls, or specimens bind to the immobilized anti-Ba monoclonal antibody. After incubation, a wash cycle removed unbound material. In step 2, horseradish peroxidase (HRP)-conjugated polyclonal anti-Factor B antibody was added to each test well. The enzyme conjugated anti-Factor B binds to Ba captured in the micro-assay wells. After incubation, a wash cycle removed unbound, excess conjugate. In step 3, a chromogenic enzyme substrate was added to each micro-assay well. The bound HRP-conjugate reacted with the substrate, forming a blue color. After incubation the enzyme reaction was stopped chemically, the color changed to yellow, and the color intensity was measured spectrophotometrically at 450 nm. The color intensity of the reaction mixture is proportional to the concentration of Ba present in the test specimens, Standards, and Controls. The results of the six patients were pooled and reported as non-normalized (FIG. 5A, Table 3 with respect to each patient individually), normalized to creatinine (FIG. 5B, Table 4 with respect to each patient individually) and normalized to albumin (FIG. 5C, Table 5 with respect to each patient individually). The reference range for “normal” levels of Ba in urine ranged from less than lower limit of quantitation (0.033 ng/ml) to 3.32 ng/ml (Table 6). Measurements of urinary biomarkers are typically normalized to urine creatinine and/or albumin levels to account for variation in biomarker levels due to changes in urinary filtration rate. Urinary Ba concentrations were elevated above normal in patient urine samples collected at baseline. Urinary Ba levels were reduced significantly during a two-week treatment with Compound 1. Urinary Ba levels normalized to urinary creatinine or albumin levels also were reduced significantly during a two-week treatment with Compound 1.

TABLE 3

| | Urine Ba ng/mL | | | | | | | | |
|---|---|---|---|---|---|---|---|---|---|
| | A | C | B | D | E | F | MEAN | GEOMEAN | N |
| Screen 1 | ND | ND | ND | ND | ND | ND | | | |
| Screen | 28.3 | 0.12 | 348 | 84.4 | ND | ND | | | |
| Day 1/0 Hour | 47.1 | 0.30 | 793 | 14.2 | 252 | 74.9 | 197 | 38.1 | 6 |
| Day 4 | 14.7 | 0.08 | 158 | 4.4 | 129 | 5.7 | 52 | 9.1 | 6 |
| Day 7/0 Hour | 11.6 | 0.21 | 149 | 2.4 | 142 | 2.1 | 51 | 7.9 | 6 |
| Day 10 | 8.6 | 0.08 | 53 | 1.8 | 106 | 2.1 | 28 | 5.0 | 6 |
| Day 14 | ND | 0.09 | 238 | 4.1 | 41 | 9.8 | | | |
| Day 15 Taper | 11.9 | 0.15 | 209 | 8.4 | 11 | 2.8 | 41 | 6.8 | 6 |
| Day 16 Taper | 17.4 | 0.15 | 318 | ND | 20 | 2.2 | | | |
| Day 17 Taper | 21.2 | 0.13 | 164 | 5.0 | 47 | 6.9 | 41 | 9.5 | 6 |
| Day 21 Taper | 79.8 | 0.49 | 129 | 18.6 | 42 | 6.6 | 46 | 17.2 | 6 |
| Follow-Up Day 24 | 115.3 | 0.14 | 251 | 28.7 | 156 | 2.2 | 92 | 18.4 | 6 |
| Follow-Up Day 28 | 100.8 | 0.62 | 175 | 30.0 | 234 | 2.8 | 90 | 24.5 | 6 |
| Follow-Up Day 35 | ND | ND | ND | ND | ND | ND | | | |
| Follow-Up Day 49 | 16.5 | 0.16 | 1727 | 9.4 | 207 | 44.4 | 334 | 27.2 | 6 |

TABLE 4

| | Ba/Cr ug/mmol | | | | | | | | |
|---|---|---|---|---|---|---|---|---|---|
| | A | C | B | D | E | F | MEAN | GEOMEAN | N |
| Screen 1 | ND | ND | ND | ND | ND | ND | | | |
| Screen | 3.4 | 0.065 | 47.1 | 11.1 | ND | ND | | | |
| Day 1/0 Hour | 5.7 | 0.168 | 129.9 | 2.5 | 14.2 | 7.6 | 26.7 | 5.7 | 6 |
| Day 4 | 1.8 | 0.040 | 12.4 | 1.0 | 6.2 | 1.2 | 3.8 | 1.4 | 6 |
| Day 7/0 Hour | 1.8 | 0.056 | 10.2 | 2.1 | 9.2 | 0.5 | 4.0 | 1.5 | 6 |
| Day 10 | 1.4 | 0.053 | 7.0 | 1.8 | 8.0 | ND | | | |
| Day 14 | ND | 0.068 | 14.2 | 1.3 | 1.8 | 1.1 | | | |
| Day 15 Taper | 1.6 | 0.099 | 19.0 | 2.0 | 1.2 | 0.5 | 4.1 | 1.2 | 6 |
| Day 16 Taper | 2.3 | 0.078 | 25.2 | ND | 2.0 | 0.6 | | | |
| Day 17 Taper | 2.1 | 0.058 | 14.5 | 1.7 | 6.0 | 0.7 | 4.2 | 1.6 | 6 |
| Day 21 Taper | 9.2 | 0.175 | 15.9 | 4.4 | 5.5 | 0.5 | 6.0 | 2.6 | 6 |
| Follow-Up Day 24 | 11.5 | 0.075 | 41.1 | 4.2 | 12.5 | 0.4 | 11.6 | 3.0 | 6 |
| Follow-Up Day 28 | 10.2 | 0.213 | 24.6 | 6.2 | 14.1 | 0.6 | 9.3 | 3.8 | 6 |
| Follow-Up Day 35 | ND | ND | ND | ND | ND | ND | | | |
| Follow-Up Day 49 | 3.8 | 0.036 | 108.0 | 2.4 | 13.1 | 8.2 | 22.6 | 3.9 | 6 |

TABLE 5

| | Ba/ALB ng/mg | | | | | | | | |
|---|---|---|---|---|---|---|---|---|---|
| | A | C | B | D | E | F | MEAN | GEOMEAN | N |
| Screen 1 | ND | ND | ND | ND | ND | ND | | | |
| Screen | 15.0 | 1.5 | 97 | 19.2 | ND | ND | | | |
| Day 1/0 Hour | 22.1 | 2.9 | 225 | 10.9 | 33.5 | 25.9 | 53.4 | 22.6 | 6 |
| Day 4 | 8.4 | 1.5 | 47 | 3.5 | 14.7 | 7.6 | 13.8 | 7.8 | 6 |
| Day 7/0 Hour | 7.5 | 2.6 | 41 | 6.5 | 17.3 | 5.7 | 13.4 | 8.9 | 6 |
| Day 10 | 6.1 | 1.5 | 28 | 2.8 | 12.8 | ND | | | |
| Day 14 | ND | 2.2 | 54 | 4.6 | 6.9 | 5.8 | | | |
| Day 15 Taper | 11.4 | 2.2 | 58 | 4.3 | 3.6 | 0.6 | 13.4 | 4.9 | 6 |
| Day 16 Taper | 16.3 | 3.0 | 77 | ND | 7.2 | 2.6 | | | |
| Day 17 Taper | 18.6 | 1.8 | 55 | 2.9 | 12.7 | 1.6 | 15.4 | 6.9 | 6 |
| Day 21 Taper | 65.5 | 7.5 | 43 | 7.8 | 21.6 | 1.5 | 24.5 | 13.2 | 6 |
| Follow-Up Day 24 | 83.3 | 2.3 | 87 | 14.9 | 41.2 | 1.5 | 38.5 | 15.7 | 6 |
| Follow-Up Day 28 | 77.1 | 4.5 | 65 | 10.8 | 46.1 | 2.5 | 34.3 | 17.4 | 6 |
| Follow-Up Day 35 | ND | ND | ND | ND | ND | ND | | | |
| Follow-Up Day 49 | 26.5 | 1.7 | 771 | 3.8 | 40.0 | 14.5 | 143.0 | 20.7 | 6 |

TABLE 6

| Health | Sample 1 | Sample 2 | Sample 3 |
|---|---|---|---|
| Control | (uBa (ng/ml)) | (uBa (ng/ml)) | (uBa (ng/ml)) |
| 1 | 2.76 | 3.05 | 3.32 |
| 2 | <LLOQ | <LLOQ | <LLOQ |
| 3 | <LLOQ | <LLOQ | <LLOQ |

To measure urinary sC5b-9 production, patient urine samples were tested utilizing the MicroVue sC5b-9 Enzyme Immunoassay for the quantitation of sC5b-9 in urine. Urine samples were collected from patients A, B, C, D, E, and F at protocol-specified timepoints prior to, during, and after dosing with Compound 1. The samples were tested utilizing the MicroVue sC5b-9 Enzyme Immunoassay for the quantitation of sC5b-9 urine, which is a three-step procedure utilizing a micro-assay plate coated with a mouse monoclonal antibody that binds specifically to the C9 ring of sC5b-9, HRP-conjugated antibodies to antigens of sC5b-9, and a chromogenic substrate. In step 1, standards, controls, and test specimens are added to micro-assay wells pre-coated with an anti-sC5b-9 specific monoclonal antibody. sC5b-9 present in the Standards, Controls, or specimens will bind to the immobilized sC5b-9 monoclonal antibody. After incubation, a wash cycle removes unbound material. Constituent proteins of the terminal complement complex (TCC), including C9, do not bind to this antibody and are washed away during the wash cycle. In step 2, horseradish peroxidase (HRP)-conjugated antibodies to antigens of sC5b-9 are added to each test well. The enzyme conjugated antibodies bind to sC5b-9 that was captured by the monoclonal anti-sC5b-9 bound on the surface of the micro-assay wells. After incubation, a wash cycle removes unbound, excess conjugate. In step 3, a chromogenic enzyme substrate is added to each micro-assay well. The bound HRP-conjugate reacts with the substrate, forming a blue color. After incubation the enzyme reaction is stopped chemically, the color changes to yellow, and the color intensity is measured spectrophotometrically at 450 nm. The color intensity of the reaction mixture is proportional to the concentration of sC5b-9 present in the test specimens, standards, and controls. The results of the six patients were pooled and reported as non-normalized (FIG. 5D, Table 7 with respect to each patient individually), normalized to creatinine (FIG. 5E, Table 8 with respect to each patient individually) and normalized to albumin (FIG. 5F, Table 9 with respect to reach patient individually). The reference range for “normal” levels of sC5b-9 levels in urine was below the lower limit of quantitation (8.8 ng/ml) (Table 10). Urinary sC5b-9 concentrations were elevated above normal in patient urine sample collected at baseline. Measurements of urinary biomarkers are typically normalized to urine creatinine or albumin levels to account for variation in biomarker levels due to changes in urinary filtration rate. Urinary sC5b-9 levels were reduced significantly during a two-week treatment with Compound 1. Urinary sC5b-9 levels normalized to urinary creatinine or albumin levels also were reduced significantly during a two-week treatment with Compound 1.

TABLE 7

| | Urine sC5b-9 ng/mL | | | | | | | | |
|---|---|---|---|---|---|---|---|---|---|
| | A | C | B | D | E | F | MEAN | GEOMEAN | N |
| Screen 1 | ND | 57.4 | ND | ND | ND | ND | | | |
| Screen | 76.3 | 32.1 | 2598 | 148.7 | ND | ND | | | |
| Day 1 predose | 81.2 | 21.5 | 5280 | 28.1 | 959 | 348 | 1120 | 210 | 6 |
| Day 4 | 84.2 | <8.8 | 2139 | 18.5 | 538 | 43 | 472 | 94 | 6 |
| Day 7 | 70.4 | <8.8 | 2266 | 15.8 | 484 | 54 | 483 | 91 | 6 |
| Day 10 | 52.0 | <8.8 | 1073 | 8.9 | 212 | 34 | 231 | 56 | 6 |
| Day 14 | ND | <8.8 | 4010 | 12.0 | 112 | 62 | | | |
| Day 15 Taper | 56.9 | <8.8 | 3334 | 20.3 | 70 | 35 | 588 | 66 | 6 |
| Day 16 Taper | 66.2 | <8.8 | 4784 | ND | 42 | 25 | | | |
| Day 17 Taper | 84.4 | <8.8 | 2693 | 15.3 | 74 | 55 | 488 | 71 | 6 |
| Day 21 Taper | 85.8 | 10.3 | 2469 | 44.0 | 97 | 104 | 468 | 99 | 6 |
| Follow-Up Day 24 | 95.0 | 11.0 | 3958 | 39.9 | 188 | 51 | 724 | 108 | 6 |
| Follow-Up Day 28 | 79.7 | 21.2 | 2376 | 57.8 | 141 | 65 | 457 | 113 | 6 |
| Follow-Up Day 35 | ND | ND | ND | ND | ND | ND | | | |
| Follow-Up Day 49 | 57.5 | 14.0 | 14132 | 40.3 | 217 | 180 | 2440 | 162 | 6 |

TABLE 8

| | sC5b-9/ALB ng/mg | | | | | | | | |
|---|---|---|---|---|---|---|---|---|---|
| | A | C | B | D | E | F | MEAN | GEOMEAN | N |
| Screen 1 | ND | 422 | ND | ND | ND | ND | | | |
| Screen | 40 | 387 | 724 | 34 | ND | ND | | | |
| Day 1/0 Hour | 38 | 203 | 1500 | 22 | 127 | 120 | 335.0 | 125.0 | 6 |
| Day 4 | 48 | 173 | 637 | 15 | 61 | 58 | 165.3 | 80.6 | 6 |
| Day 7/0 Hour | 46 | 110 | 624 | 43 | 59 | 147 | 171.5 | 102.8 | 6 |
| Day 10 | 37 | 160 | 563 | 14 | 26 | ND | | | |
| Day 14 | ND | 220 | 911 | 13 | 19 | 37 | | | |
| Day 15 Taper | 54 | 131 | 929 | 10 | 23 | 8 | 192.6 | 48.2 | 6 |
| Day 16 Taper | 62 | 176 | 1154 | ND | 15 | 30 | | | |
| Day 17 Taper | 74 | 126 | 907 | 9 | 20 | 12 | 191.3 | 51.5 | 6 |
| Day 21 Taper | 70 | 158 | 821 | 18 | 50 | 24 | 190.1 | 76.3 | 6 |
| Follow-Up Day 24 | 69 | 186 | 1380 | 21 | 50 | 34 | 289.8 | 92.3 | 6 |
| Follow-Up Day 28 | 61 | 154 | 882 | 21 | 28 | 57 | 200.5 | 80.5 | 6 |
| Follow-Up Day 35 | ND | ND | ND | ND | ND | ND | | | |
| Follow-Up Day 49 | 92 | 149 | 6312 | 16 | 42 | 59 | 1111.7 | 123.3 | 6 |

TABLE 9

| | sC5b-9/Cr ug/mmol | | | | | | | | |
|---|---|---|---|---|---|---|---|---|---|
| | A | C | B | D | E | F | MEAN | GEOMEAN | N |
| Screen 1 | ND | 52.2 | ND | ND | ND | ND | | | |
| Screen | 9.2 | 16.9 | 351 | 19.6 | ND | ND | | | |
| Day 1/0 Hour | 9.9 | 11.9 | 866 | 4.8 | 54.0 | 35.2 | 163.6 | 31.3 | 6 |
| Day 4 | 10.5 | 4.63 | 168 | 4.1 | 25.7 | 8.8 | 37.0 | 14.0 | 6 |
| Day 7/0 Hour | 10.7 | 2.38 | 155 | 14.3 | 31.5 | 13.9 | 38.0 | 17.1 | 6 |
| Day 10 | 8.4 | 5.50 | 143 | 8.9 | 16.1 | ND | | | |
| Day 14 | ND | 6.77 | 240 | 3.9 | 5.0 | 6.7 | | | |
| Day 15 Taper | 7.8 | 5.87 | 303 | 4.8 | 7.5 | 5.8 | 55.8 | 11.9 | 6 |
| Day 16 Taper | 8.8 | 4.63 | 380 | ND | 4.2 | 7.0 | | | |
| Day 17 Taper | 8.5 | 4.00 | 238 | 5.3 | 9.4 | 5.9 | 45.3 | 11.6 | 6 |
| Day 21 Taper | 9.9 | 3.67 | 305 | 10.5 | 12.7 | 8.2 | 58.3 | 15.2 | 6 |
| Follow-Up Day 24 | 9.5 | 6.11 | 649 | 5.9 | 15.1 | 9.5 | 115.8 | 17.8 | 6 |
| Follow-Up Day 28 | 8.0 | 7.32 | 335 | 12.0 | 8.5 | 14.4 | 64.2 | 17.6 | 6 |
| Follow-Up Day 35 | ND | ND | ND | ND | ND | ND | | | |
| Follow-Up Day 49 | 13.4 | 3.04 | 883 | 10.3 | 13.7 | 33.2 | 159.5 | 23.5 | 6 |

TABLE 10

| Health | Sample 1 | Sample 2 | Sample 3 |
|---|---|---|---|
| Control | (usC5b-9 (ng/ml)) | (usC5b-9 (ng/ml)) | (usC5b-9 (ng/ml)) |
| 1 | <LLOQ | ,<LLOQ | <LLOQ |
| 2 | <LLOQ | <LLOQ | <LLOQ |
| 3 | <LLOQ | <LLOQ | <LLOQ |

### Example 5. Urinary Complement Proteins: Selected Individual Patients

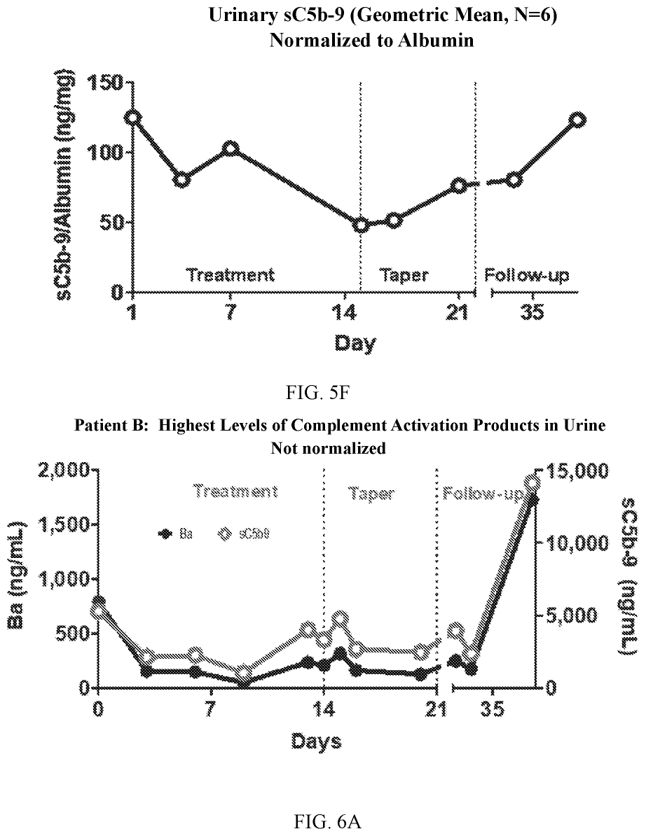

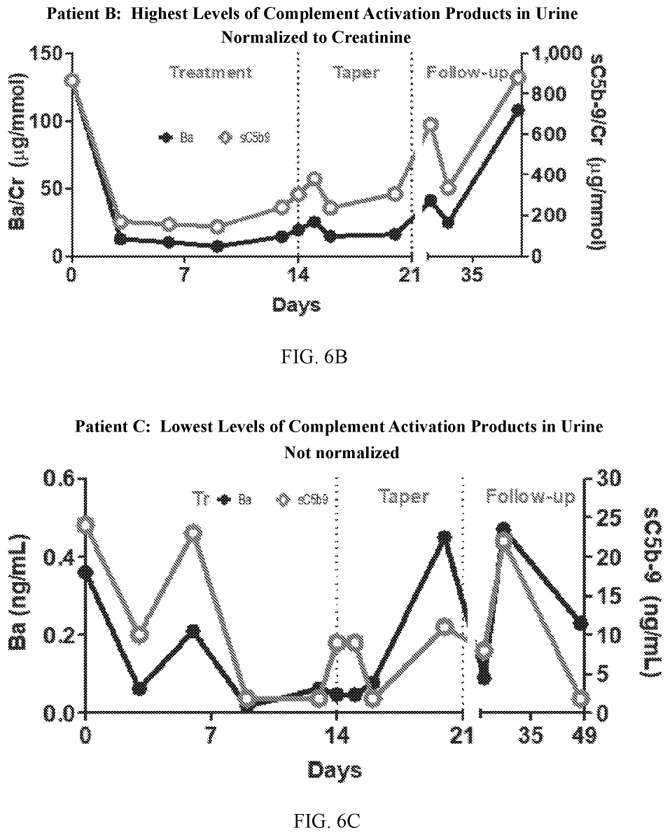

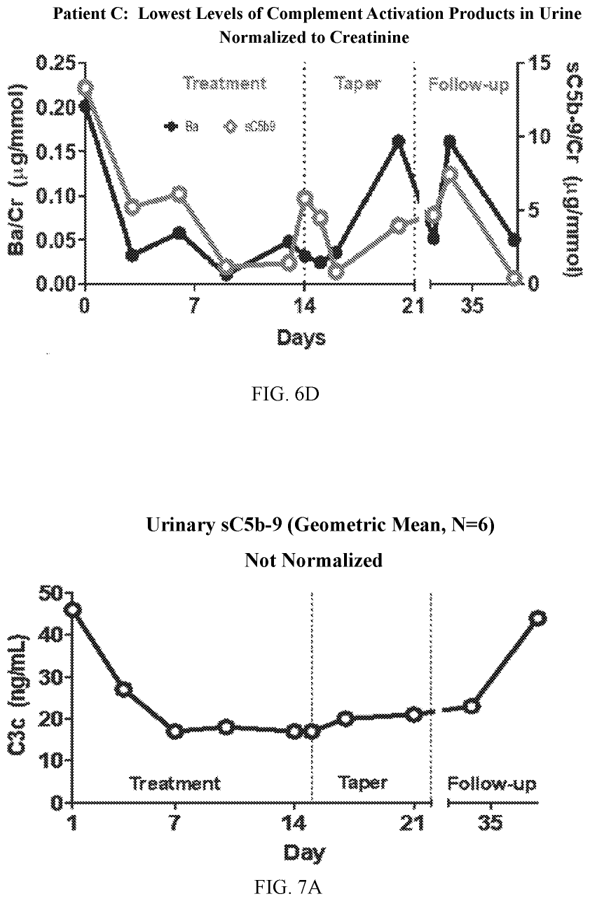

In two patients, patient B and patient C, urinary Ba and sC5b-9 levels were measured from urine samples collected at protocol-specified timepoints prior to, during, and after dosing with Compound 1. Urinary Ba and sC5b-9 levels were determined using the methods described above. Results were shown in patient B representing the highest levels of complement activation products in urine. The non-normalized results are shown in FIG. 6A and the results normalized to creatinine are shown in FIG. 6B. Results were shown in patient C representing the lowest levels of complement activation products in urine. The non-normalized results are shown in FIG. 6C and the results normalized to creatinine are shown in FIG. 6D. Absolute, creatinine-normalized, and albumin-normalized urinary Ba and sC5b-9 levels varied widely at baseline among patients. Patient B had the highest degree of proteinuria and also the highest absolute and normalized urinary Ba and sC5b-9 levels at baseline. Patient C had the lowest degree of proteinuria and also the lowest absolute and normalized urinary Ba and sC5b-9 levels at baseline. Regardless of baseline levels, urinary Ba and sC5b-9 levels were reduced in all patients during a two-week treatment with Compound 1.

### Example 6. Measuring C3c in Urine Samples

To measure urinary C3c production, patient urine samples were tested utilizing the Hycult Biotech Human C3c Elisa Kit (HK368) for the quantitation of C3c in urine. Urine samples were collected from patients A, B, C, D, E, and F at protocol-specified timepoints prior to, during, and after dosing with Compound 1. Urine samples were thawed and centrifuged at 15,000 rpm for one minute. The supernatant was transferred to a fresh tube and diluted with C3c dilution buffer. Samples and standards were added to microtiter wells coated with antibodies recognizing human C3c and incubated at 1 hour at 15-25° C. Following incubation, the wells were washed 4 times with Wash Solution, with an incubation period of 1 minute each time. A biotinylated tracer antibody was added and used to bind to the captured human C3c. Streptavidin-peroxidase conjugate was then added to bind to the biotinylated tracer antibody. Tetramethylbenzidine (TMB) was then added to the Streptavidin-peroxidase conjugate. The enzyme reaction was stopped by the addition of oxalic acid. The absorbance at 450 nm was measured with a spectrophotometer. A standard curve was obtained by plotting the absorbance (linear) versus the corresponding concentrations of the human C3c standards (log). The human C3c concentration of samples, which are run concurrently with the standards, were determined from the standard curve.

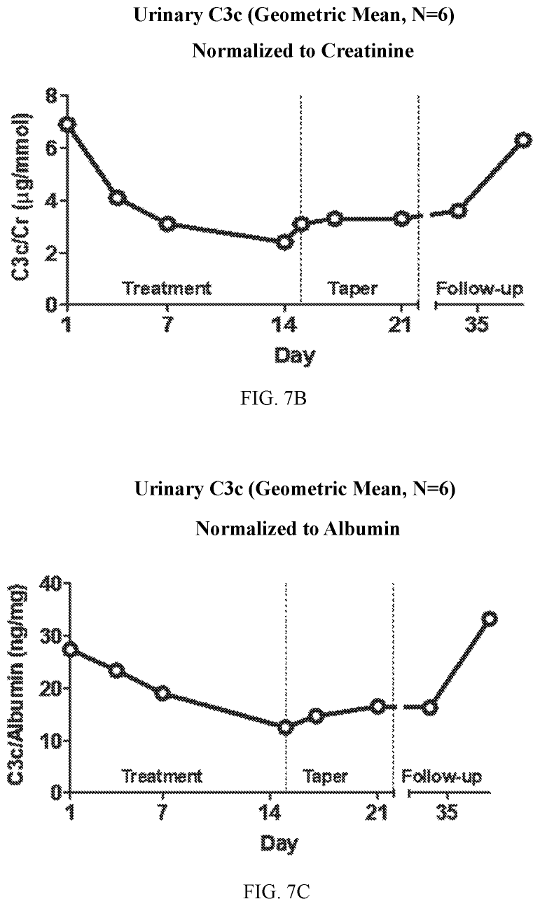

The C3c urine “normal” reference range levels in 7 individuals without any signs of kidney disease (“healthy control”) were determined as described above, and show a C3c ng/ml urine level of <LLOQ (1.6 ng/ml) (Table 11). The results of the six patients were pooled and reported as non-normalized (FIG. 7A, Table 12 with respect to each patient individually), normalized to creatinine (FIG. 7B), and normalized to albumin (FIG. 7C). Urinary C3c concentrations were elevated above normal in patient urine sample collected at baseline. Measurements of urinary biomarkers are typically normalized to urine creatinine or albumin levels to account for variation in biomarker levels due to changes in urinary filtration rate. Urinary C3c levels were reduced significantly during a two-week treatment with Compound 1. Urinary C3c levels normalized to urinary creatinine or albumin levels also were reduced significantly during a two-week treatment with Compound 1.

TABLE 11
Normal Urine

| Healthy Control | 1 | 2 | 3 | 4 | 5 | 6 | 7 |
|---|---|---|---|---|---|---|---|
| uC3c (ng/ml) | <LLOQ | <LLOQ | <LLOQ | <LLOQ | <LLOQ | <LLOQ | <LLOQ |

TABLE 12

| | uC3c ng/mL | | | | | |
|---|---|---|---|---|---|---|
| | A | C | B | D | E | F |
| Screen | ND | ND | ND | ND | ND | ND |
| Day 1 Predose | 25.4 | <1.6 | 771.1 | 8.8 | 159.6 | 216.3 |
| Day 4 | 15.2 | <1.6 | 369.0 | 5.3 | 137.7 | 63.0 |
| Day 7-0 Hour | 7.1 | <1.6 | 289.2 | <1.6 | 168.2 | 25.8 |
| Day 10 | 9.9 | <1.6 | 256.0 | <1.6 | 123.2 | 43.9 |
| Day 14 | 3.6 | <1.6 | 462.7 | 3.7 | 64.4 | 31.8 |
| Day 15 Taper | 6.8 | <1.6 | 682.8 | 3.6 | 39.7 | 24.2 |
| Day 16 Taper | 4.1 | ND | 534.1 | ND | 46.8 | 30.0 |
| Day 17 Taper | 8.5 | <1.6 | 290.8 | 4.8 | 107.0 | 32.4 |
| Day 21 | 7.8 | <1.6 | 513.5 | 5.8 | 41.2 | 64.0 |
| Day 24 | 11.4 | 2.1 | 744.1 | 5.1 | 89.6 | 34.4 |
| Day 28 | 15.6 | <1.6 | 285.3 | 5.1 | 97.8 | 41.5 |
| Day 35 | ND | ND | ND | ND | ND | ND |
| Day 49 | 32.8 | <1.6 | >1000 | 7.0 | 120.3 | 154.5 |

This specification has been described with reference to embodiments of the invention. The invention has been described with reference to assorted embodiments, which are illustrated by the accompanying Examples. The invention can, however, be embodied in different forms and should not be construed as limited to the embodiments set forth herein. Given the teaching herein, one of ordinary skill in the art will be able to modify the invention for a desired purpose and such variations are considered within the scope of the invention.

We claim:

1. A method for the diagnosing a human subject having a component 3 glomerulopathy (C3 G) disorder or a membranoproliferative glomerulonephritis (MPGN) disorder comprising:
i. analyzing the levels in the urine of the subject of a biomarker selected from Ba, sC5b-9, C3c, or a combination thereof;
ii. comparing the subject's urine biomarker levels to a range of the same urine biomarker levels derived from individuals that do not have a C3G disorder or an MPGN disorder (“normal range”); and
iii. if the subject's urine biomarker levels are greater than the normal range, diagnosing the subject with a C3G disorder or an MPGN disorder.

2. A method for the targeted selection and treatment of a human subject suffering from a component 3 glomerulopathy (C3G) disorder or a membranoproliferative glomerulonephritis (MPGN) disorder comprising:
i. analyzing the levels in the urine of the subject of a biomarker selected from Ba, sC5b-9, C3c, or a combination thereof;
ii. comparing the subject's urine biomarker levels to a range of the same urine biomarker levels derived from individuals that do not have a C3G disorder or MPGN disorder (“normal range”); and
iii. if the subject's urine biomarker levels are greater than the normal range, diagnosing the subject with a C3G disorder or an MPGN disorder and administering to the subject a complement alternative pathway (AP) inhibitor.

3. The method of claim 1 or 2, wherein the subject's urine biomarker levels are at least 2×, 3×, 4×, 5×, 6×, 7×, 8×, 9×, or 10× higher than the upper limit of the normal range.

4. The method of claim 1 or 2, wherein the subject's urine biomarker levels are at least 100×, 200×, 300×, 400×, or 500× higher than the upper limit of the normal range.

5. A method for the targeted selection and treatment of a human subject suffering from a component 3 glomerulopathy (C3G) disorder or a membranoproliferative glomerulonephritis (MPGN) disorder comprising:
i. analyzing the levels in the urine of the subject of a biomarker selected from Ba, sC5b-9, C3c, or a combination thereof;
ii. comparing the subject's urine biomarker levels to a range of urine biomarker levels derived from individuals that have a C3G disorder or an MPGN disorder (“abnormal range”); and
iii. if the subject's urine biomarker levels fall within the abnormal range, diagnosing the subject with a C3G disorder or an MPGN disorder.

6. A method for the targeted selection and treatment of a human subject suffering from a component 3 glomerulopathy (C3G) disorder or a membranoproliferative glomerulonephritis (MPGN) disorder comprising:
i. analyzing the levels in the urine of the subject of a biomarker selected from Ba, sC5b-9, C3c, or a combination thereof;
ii. comparing the subject's urine biomarker levels to a range of urine biomarker levels derived from individuals that have a C3G disorder or an MPGN disorder (“abnormal range”); and
iii. if the subject's urine biomarker levels fall within the abnormal range, diagnosing the subject with a C3G disorder or an MPGN disorder and administering to the subject an AP inhibitor.

7. A method of monitoring the efficacy of an alternative pathway (AP) inhibitor treatment regimen in a human subject having a component 3 glomerulopathy (C3G) disorder or a membranoproliferative glomerulonephritis (MPGN) disorder and receiving an AP inhibitor comprising:
i. analyzing the levels in a first urine sample from the subject of a biomarker selected from Ba, sC5b-9, C3c, or a combination thereof;
ii. analyzing levels of the urine biomarkers in a subsequent urine sample from the subject, wherein the first urine sample is collected prior to the collection of the subsequent urine sample;
iii. comparing the biomarker levels in the first urine sample to the biomarker levels in the subsequent urine sample; and
iv. if the biomarker levels in the subsequent urine sample are equal to or greater than the biomarker levels in the first urine sample, then increasing the dose of the AP inhibitor being administered to the subject.

8. A method of monitoring the efficacy of an alternative pathway (AP) inhibitor treatment regimen in a human subject having a component 3 glomerulopathy (C3G) disorder or a membranoproliferative glomerulonephritis (MPGN) disorder and receiving an AP inhibitor comprising:
i. analyzing the levels in a first urine sample from the subject of a biomarker selected from Ba, sC5b-9, C3c, or a combination thereof;
iii. analyzing levels of the urine biomarkers in a subsequent urine sample from the subject, wherein the first urine sample is collected prior to the collection of the subsequent urine sample;
ii. comparing the biomarker levels in the first urine sample to the biomarker levels in the subsequent urine sample; and
iv. if the biomarker levels in the subsequent urine sample have insufficiently decreased compared to the biomarker levels in the first urine sample, then increasing the dose of the AP inhibitor being administered to the subject.

9. The method of claim 8, wherein the biomarker levels in the subsequent urine sample have decreased by less than 10%, 20%, 30%, 40%, 50%, 60%, 70%, 80%, or 90% of the biomarker levels in the first urine sample.

10. A method for the prediction of a long-term benefit of a human subject suffering from component 3 glomerulopathy (C3G) disorder or a membranoproliferative glomerulonephritis (MPGN) disorder comprising:
i. treating the subject suffering from a C3G disorder or a MPGN disorder with an AP inhibitor regimen;
ii. analyzing the levels in the urine of the subject of a biomarker selected from Ba, sC5b-9, and C3c, or a combination thereof; and
iii. maintaining the subject's urine biomarker levels within a range of the urine biomarker levels derived from individuals that do not have C3G disorder or an MPGN disorder (“normal range”).

11. The method of any one of claims 1-10, wherein the biomarker is Ba.

12. The method of any one of claims 1-10, wherein the biomarker is sC5b-9.

13. The method of any one of claims 1-10, wherein the biomarker is C3c.

14. The method of any one of claims 1-10, wherein the biomarkers are Ba and sC5b-9.

15. The method of any one of claims 1-10, wherein the biomarkers are Ba and C3c.

16. The method of any one of claims 1-10, wherein the biomarkers are sC5b-9 and C3c.

17. The method of any one of claims 1-10, wherein the biomarkers are Ba, sC5b-9, and C3c.

18. The method of any one of claims 1-17, wherein the biomarker levels are normalized.

19. The method of claim 16, wherein the biomarker levels are normalized to urine creatinine.

20. The method of claim 19, wherein the biomarker levels are normalized to urine albumin.

21. The method of claim 19, wherein the biomarker levels are normalized to both urine albumin and urine creatinine.

22. The method of any one of claim 2-4, or 6-21, wherein the AP inhibitor is a factor D (fD) inhibitor.

23. The method of claim 22, wherein the fD inhibitor is selected from BioCryst Pharmaceuticals fD inhibitor, a Novartis fD inhibitor, a Bristol-Myers Squibb fD inhibitor, a Japan Tobacco Inc. fD inhibitor, FCFD4515S, nafomostat, SOMAmers for fD (SomaLogic), lampalizumab, aptamers to fD (Vitrisia Therapeutics), a Ra Pharmaceutical fD inhibitor, an Alexion Pharmaceuticals fD inhibitor, and an Achillion Pharmaceuticals fD inhibitor.

24. The method of claim 22, wherein the fD inhibitor is a compound of Formula I: 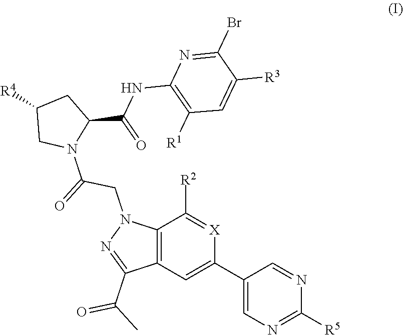
or a pharmaceutically acceptable salt, N-oxide, isotopic derivative, or prodrug thereof;
wherein:
X is selected from N and CH;
R¹ is selected from hydrogen, C₁-C₃ alkyl, and halogen;
R² is selected from hydrogen and C₁-C₃ alkyl;
R³ is selected from hydrogen, C₁-C₃ alkyl, and halogen;
R⁴ is selected from hydrogen, C₁-C₃ alkyl, and halogen; and
R⁵ is selected from hydrogen, C₁-C₃ alkyl, halogen, and cyano.

25. The method of claim 24, wherein the compound is selected from:

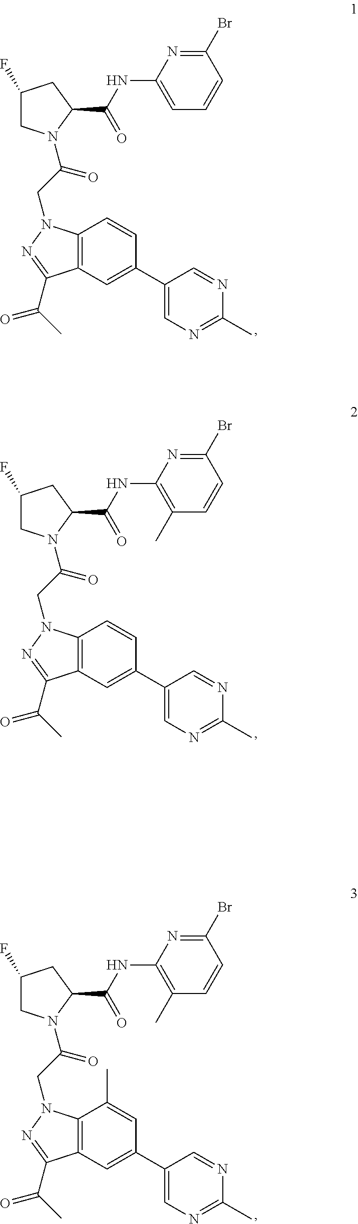 [SMILES: CC(=O)c1nn(CC(=O)N2C[C@H](F)C[C@H]2C(=O)Nc2cccc(Br)n2)c2ccc(-c3cnc(C)nc3)cc12 ; CC(=O)c1nn(CC(=O)N2C[C@H](F)C[C@H]2C(=O)Nc2nc(Br)ccc2C)c2ccc(-c3cnc(C)nc3)cc12 ; CC(=O)c1nn(CC(=O)N2C[C@H](F)C[C@H]2C(=O)Nc2nc(Br)ccc2C)c2c(C)cc(-c3cnc(C)nc3)cc12]

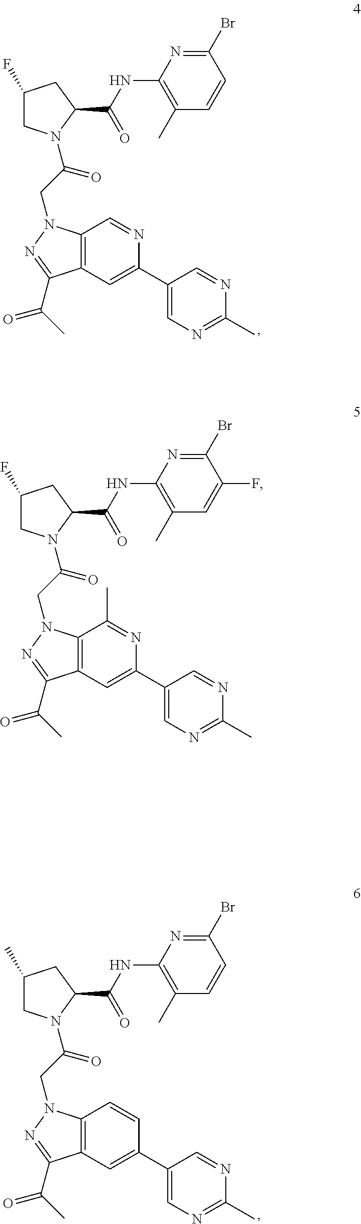

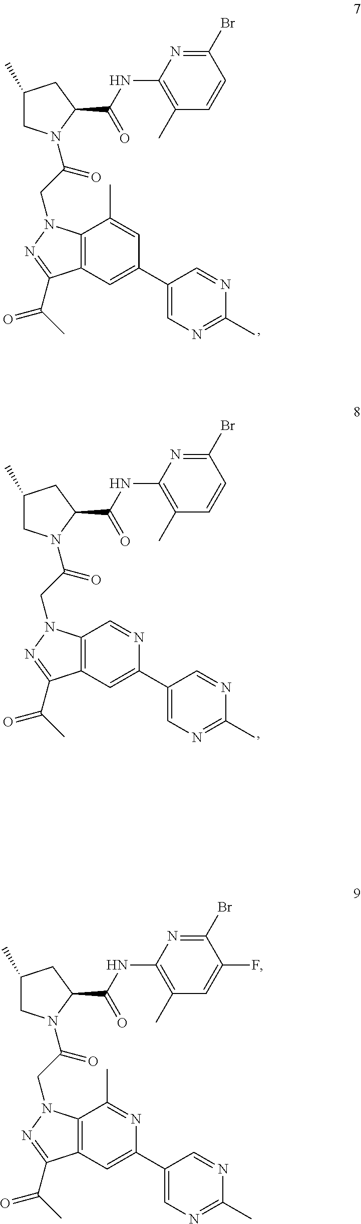

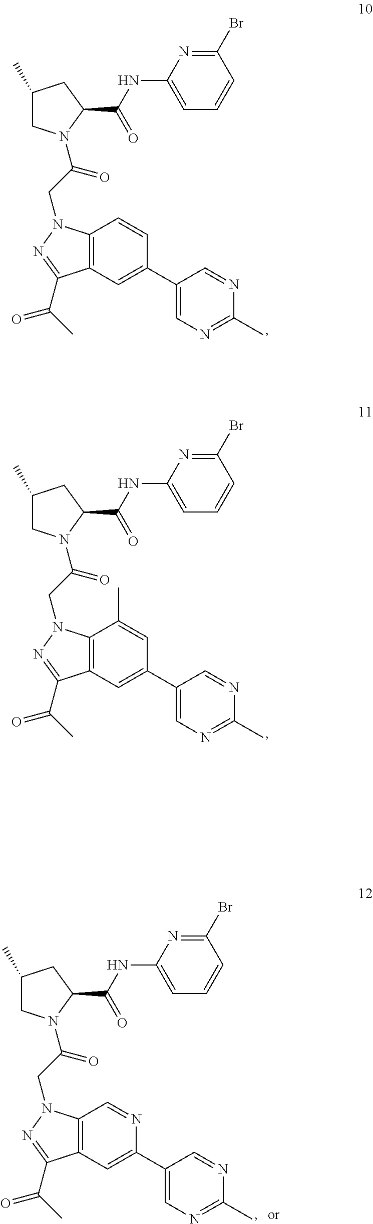

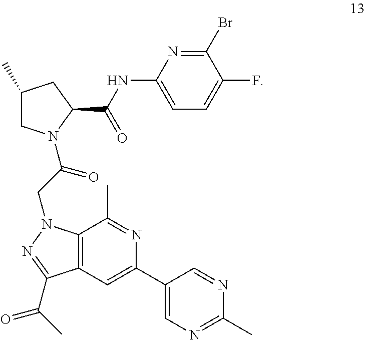 [SMILES: CC(=O)c1nn(CC(=O)N2C[C@H](C)C[C@H]2C(=O)Nc2ccc(F)c(Br)n2)c2c(C)nc(-c3cnc(C)nc3)cc12]

26. The method of claim 22, wherein the fD inhibitor is a compound of Formula II: 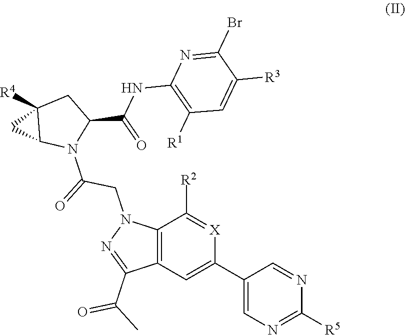
or a pharmaceutically acceptable salt, N-oxide, isotopic derivative, or prodrug thereof;
wherein:
X is selected from N and CH;
R¹ is selected from hydrogen, C₁-C₃ alkyl, and halogen;
R² is selected from hydrogen and C₁-C₃ alkyl;
R³ is selected from hydrogen, C₁-C₃ alkyl, and halogen;
R⁴ is selected from hydrogen, C₁-C₃ alkyl, and halogen; and
R⁵ is selected from hydrogen, C₁-C₃ alkyl, halogen, and cyano.

27. The method of claim 26, wherein the compound is selected from:

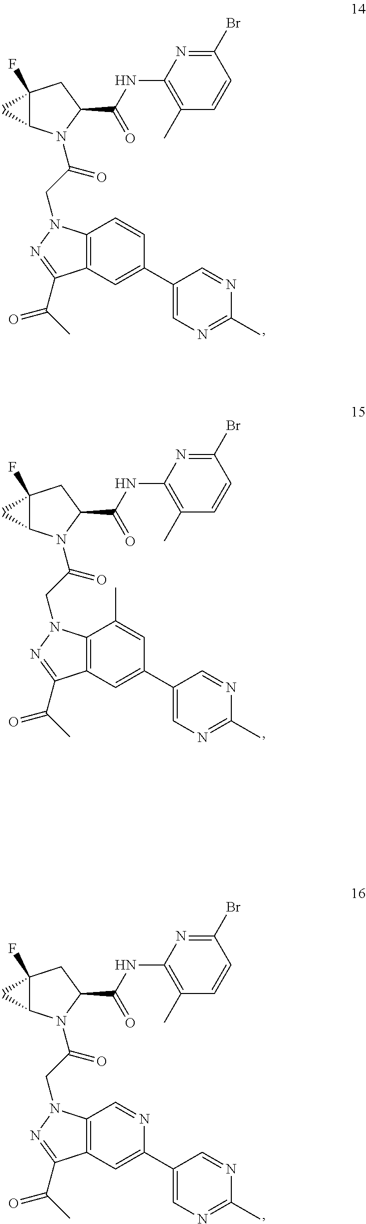 [SMILES: CC(=O)c1nn(CC(=O)N2[C@H](C(=O)Nc3nc(Br)ccc3C)C[C@@]3(F)C[C@@H]23)c2ccc(-c3cnc(C)nc3)cc12 ; CC(=O)c1nn(CC(=O)N2[C@H](C(=O)Nc3nc(Br)ccc3C)C[C@@]3(F)C[C@@H]23)c2c(C)cc(-c3cnc(C)nc3)cc12 ; CC(=O)c1nn(CC(=O)N2[C@H](C(=O)Nc3nc(Br)ccc3C)C[C@@]3(F)C[C@@H]23)c2cnc(-c3cnc(C)nc3)cc12]

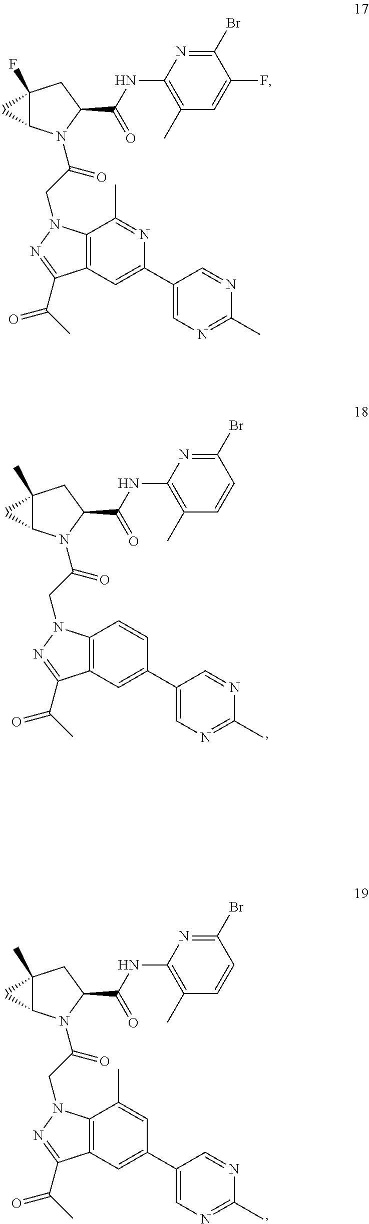 [SMILES: CC(=O)c1nn(CC(=O)N2[C@H](C(=O)Nc3nc(Br)c(F)cc3C)C[C@@]3(F)C[C@@H]23)c2c(C)nc(-c3cnc(C)nc3)cc12 ; CC(=O)c1nn(CC(=O)N2[C@H](C(=O)Nc3nc(Br)ccc3C)C[C@@]3(C)C[C@@H]23)c2ccc(-c3cnc(C)nc3)cc12 ; CC(=O)c1nn(CC(=O)N2[C@H](C(=O)Nc3nc(Br)ccc3C)C[C@@]3(C)C[C@@H]23)c2c(C)cc(-c3cnc(C)nc3)cc12]

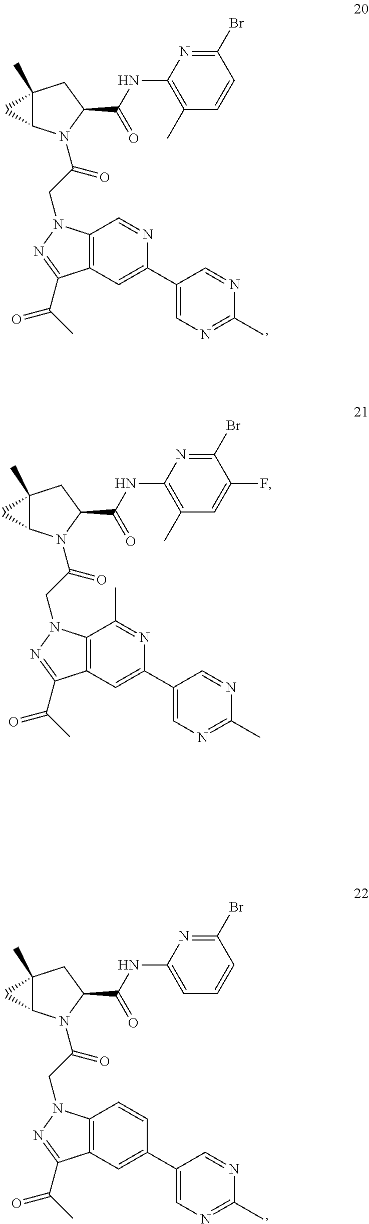 [SMILES: CC(=O)c1nn(CC(=O)N2[C@H](C(=O)Nc3nc(Br)ccc3C)C[C@@]3(C)C[C@@H]23)c2cnc(-c3cnc(C)nc3)cc12 ; CC(=O)c1nn(CC(=O)N2[C@H](C(=O)Nc3nc(Br)c(F)cc3C)C[C@@]3(C)C[C@@H]23)c2c(C)nc(-c3cnc(C)nc3)cc12 ; CC(=O)c1nn(CC(=O)N2[C@H](C(=O)Nc3cccc(Br)n3)C[C@@]3(C)C[C@@H]23)c2ccc(-c3cnc(C)nc3)cc12]

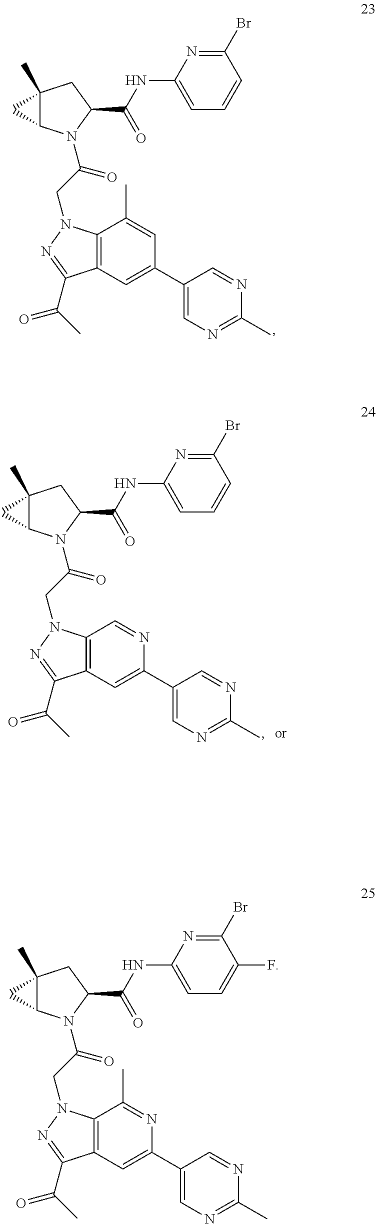 [SMILES: CC(=O)c1nn(CC(=O)N2[C@H](C(=O)Nc3cccc(Br)n3)C[C@@]3(C)C[C@@H]23)c2c(C)cc(-c3cnc(C)nc3)cc12 ; CC(=O)c1nn(CC(=O)N2[C@H](C(=O)Nc3cccc(Br)n3)C[C@@]3(C)C[C@@H]23)c2cnc(-c3cnc(C)nc3)cc12 ; CC(=O)c1nn(CC(=O)N2[C@H](C(=O)Nc3ccc(F)c(Br)n3)C[C@@]3(C)C[C@@H]23)c2c(C)nc(-c3cnc(C)nc3)cc12]

28. The method of any one of claim 2-4 or 6-21, wherein the AP inhibitor is a factor B (fB) inhibitor.

29. The method of claim 28, wherein the fB inhibitor is selected from anti-FB siRNA, TA106, LNP106, LNP023, complin, and Ionis-FB-L_{Rx}.

30. The method of claim 28, wherein the fB inhibitor is a compound of the formula:

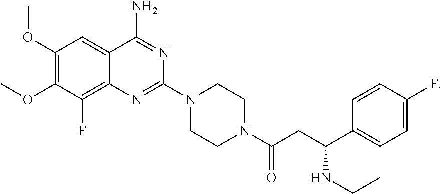 [SMILES: CCN[C@H](CC(=O)N1CCN(c2nc(N)c3cc(OC)c(OC)c(F)c3n2)CC1)c1ccc(F)cc1]

31. The method of any one of claim 2-4 or 6-21, wherein the AP inhibitor is a complement component 3 (C3) inhibitor.

32. The method of claim 31, wherein the C3 inhibitor is selected from compstatin, 4(1MeW)/APL-1, Cp40/AMY-101, PEG-Cp40, 4(1MeW) POT-4, and AMY-201.

33. The method of claim 31, wherein the C3 inhibitor is selected from selected from H17, mirocept, sCR1, TT32, HC-1496, CB-2782, and APL-2.

34. The method of any one of claim 2-4 or 6-21, wherein the AP inhibitor is a C3 convertase inhibitor.

35. The method of claim 34, wherein the C3 convertase inhibitor is selected from CRIg/CFH, Mini-CFH, TT30, and rFH (Optherion).

36. The method of any one of claim 2-4 or 6-21, wherein the AP inhibitor is a complement component 3b (C3b) inhibitor.

37. The method of claim 36, wherein the C3b inhibitor is selected from APL-2, 4(1MeW) POT-4, PEG-Cp40, H17, ALXN1102/ALXN1103, and rFH.

38. The method of any one of claims 1-37, wherein the C3G disorder is C3 glomerulonephritis (C3GN).

39. The method of any one of claims 1-37, wherein the C3G disorder is dense deposit disease (DDD).

40. The method of any one of claims 1-37, wherein the MPGN disorder is immune complex membranoproliferative glomerulonephritis (IC-MPGN).

41. A method for the targeted selection and treatment of a human subject suffering from a suspected alternative pathway (AP)-associated nephropathy selected from C3 glomerulonephritis (C3GN), dense deposit disease (DDD), and immune complex membranoproliferative glomerulonephritis (IC-MPGN) comprising:
i. analyzing the levels in the urine of the subject of a biomarker selected from Ba, sC5b-9, C3c, or a combination thereof;
ii. comparing the subject's urine biomarker levels to a range of the same urine biomarker levels derived from individuals that do not have the AP-associated nephropathy (“normal range”); and
iii. if the subject's urine biomarker levels are greater than the normal range, administering to the subject an effective amount of Compound 1: 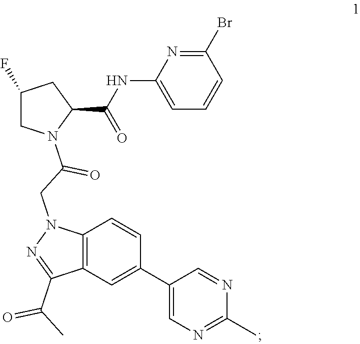 [SMILES: CC(=O)c1nn(CC(=O)N2C[C@H](F)C[C@H]2C(=O)Nc2cccc(Br)n2)c2ccc(-c3cnc(C)nc3)cc12]
or a pharmaceutically acceptable salt thereof.

42. The method of claim 41, wherein the subject's urine biomarker levels are at least 2×, 3×, 4×, 5×, 6×, 7×, 8×, 9×, or 10× higher than the upper limit of the normal range.

43. The method of claim 41, wherein the subject's urine biomarker levels are at least 100×, 200×, 300×, 400×, or 500× higher than the upper limit of the normal range.

44. A method for the targeted selection and treatment of a human subject suffering from a suspected alternative pathway (AP)-associated nephropathy selected from C3 glomerulonephritis (C3GN), dense deposit disease (DDD), and immune complex membranoproliferative glomerulonephritis (IC-MPGN) comprising:
i. analyzing the levels in the urine of the subject of a biomarker selected from Ba, sC5b-9, C3c, or a combination thereof;
ii. comparing the subject's urine biomarker levels to a range of urine biomarker levels derived from individuals that have the AP-associated nephropathy (“abnormal range”); and
iii. if the subject's urine biomarker levels fall within the abnormal range, administering to the subject an effective amount of Compound 1: 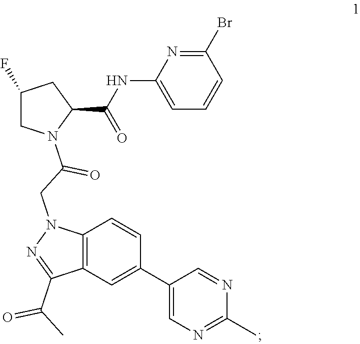 [SMILES: CC(=O)c1nn(CC(=O)N2C[C@H](F)C[C@H]2C(=O)Nc2cccc(Br)n2)c2ccc(-c3cnc(C)nc3)cc12]
or a pharmaceutically acceptable salt thereof.

45. A method of monitoring the efficacy of a treatment regimen of Compound 1: 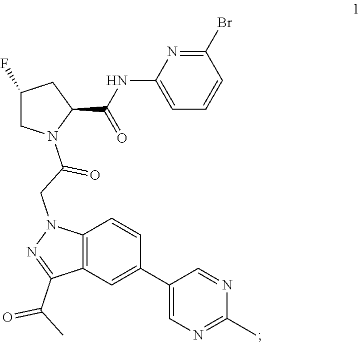 [SMILES: CC(=O)c1nn(CC(=O)N2C[C@H](F)C[C@H]2C(=O)Nc2cccc(Br)n2)c2ccc(-c3cnc(C)nc3)cc12]
or a pharmaceutically acceptable salt thereof in a human subject having an AP-associated nephropathy selected from C3 glomerulonephritis (C3GN), dense deposit disease (DDD), and immune complex membranoproliferative glomerulonephritis (IC-MPGN) and receiving Compound 1 comprising:
i. analyzing the levels in a first urine sample from the subject of a biomarker selected from Ba, sC5b-9, C3c, or a combination thereof;
ii. analyzing levels of the urine biomarkers in a subsequent urine sample from the subject, wherein the first urine sample is collected prior to the collection of the subsequent urine sample;
iii. comparing the biomarker levels in the first urine sample to the biomarker levels in the subsequent urine sample; and
iv. if the biomarker levels in the subsequent urine sample are equal to or greater than the biomarker levels in the first urine sample, then increasing the dose of Compound 1 being administered to the subject.

46. A method of monitoring the efficacy of a treatment regimen of Compound 1: 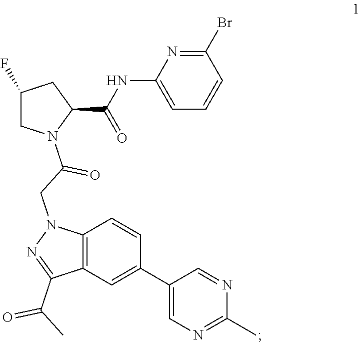 [SMILES: CC(=O)c1nn(CC(=O)N2C[C@H](F)C[C@H]2C(=O)Nc2cccc(Br)n2)c2ccc(-c3cnc(C)nc3)cc12]
or a pharmaceutically acceptable salt thereof in a human subject having an AP-associated nephropathy selected from C3 glomerulonephritis (C3GN), dense deposit disease (DDD), and immune complex membranoproliferative glomerulonephritis (IC-MPGN) and receiving Compound 1 comprising:
i. analyzing the levels in a first urine sample from the subject of a biomarker selected from Ba, sC5b-9, C3c, or a combination thereof;
iii. analyzing levels of the urine biomarkers in a subsequent urine sample from the subject, wherein the first urine sample is collected prior to the collection of the subsequent urine sample;
ii. comparing the biomarker levels in the first urine sample to the biomarker levels in the subsequent urine sample; and
iv. if the biomarker levels in the subsequent urine sample have insufficiently decreased compared to the biomarker levels in the first urine sample, then increasing the dose of Compound 1 being administered to the subject.

47. The method of claim 46, wherein the biomarker levels in the subsequent urine sample have decreased by less than 10%, 20%, 30%, 40%, 50%, 60%, 70%, 80%, or 90% of the biomarker levels in the first urine sample.

48. A method for the prediction of a long-term benefit of a human subject suffering from an alternative pathway (AP)-associated nephropathy selected from C3 glomerulonephritis (C3GN), dense deposit disease (DDD), and immune complex membranoproliferative glomerulonephritis (IC-MPGN) comprising:
i. treating the subject suffering from the AP-associated nephropathy with a regimen of Compound 1: 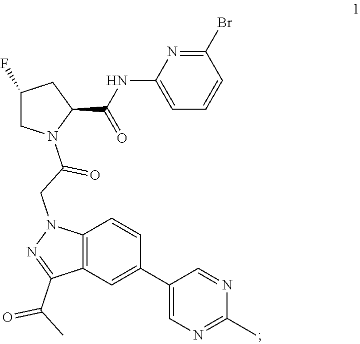 [SMILES: CC(=O)c1nn(CC(=O)N2C[C@H](F)C[C@H]2C(=O)Nc2cccc(Br)n2)c2ccc(-c3cnc(C)nc3)cc12]
or a pharmaceutically acceptable salt thereof;
ii. analyzing the levels in the urine of the subject of a biomarker selected from Ba, sC5b-9, and C3c, or a combination thereof; and
iii. maintaining the subject's urine biomarker levels within a range of the urine biomarker levels derived from individuals that do not have the AP-associated nephropathy (“normal range”).

49. The method of any one of claims 41-48, wherein the biomarker is Ba.

50. The method of any one of claims 41-48, wherein the biomarker is sC5b-9.

51. The method of any one of claims 41-48, wherein the biomarker is C3c.

52. The method of any one of claims 41-48, wherein the biomarkers are Ba and sC5b-9.

53. The method of any one of claims 41-48, wherein the biomarkers are Ba and C3c.

54. The method of any one of claims 41-48, wherein the biomarkers are sC5b-9 and C3c.

55. The method of any one of claims 41-48, wherein the biomarkers are Ba, sC5b-9, and C3c.

56. The method of any one of claims 41-55, wherein the biomarker levels are normalized.

57. The method of claim 56, wherein the biomarker levels are normalized to urine creatinine.

58. The method of claim 56, wherein the biomarker levels are normalized to urine albumin.

59. The method of claim 56, wherein the biomarker levels are normalized to both urine albumin and urine creatinine.

60. A method of treating a subject with an alternative pathway (AP)-associated nephropathy comprising administering to the subject a fD inhibitor; wherein the subject at the time of administration of the fD inhibitor has been, or is currently, receiving a therapeutic regimen comprising the administration of a C5 inhibitor; and wherein the Ba level in the subject's urine is greater than the range of Ba urine levels derived from individuals that do not have an AP-associated nephropathy.

61. The method of claim 60, wherein the Ba level in the subject's urine is at least 2×, 3×, 4×, 5×, 6×, 7×, 8×, 9×, or 10× higher than the upper limit of the range of Ba urine levels derived from individuals that do not have an AP-associated nephropathy.

62. The method of claim 60, wherein the Ba level in the subject's urine is at least 100×, 200×, 300×, 400×, or 500× higher than the upper limit of the range of Ba urine levels derived from individuals that do not have an AP-associated nephropathy.

63. The method of any one of claims 60-62, wherein the C5 inhibitor is eculizumab or ravulizumab-Cwvz.

64. The method of any one of claims 60-62, wherein the C5 inhibitor is selected from a recombinant human minibody for C5, coversin, Tesidolumab/LFG316, ARC-1905, RA101348, RA101495, SOBI002, ARC1005, a SOMAmer for C5, SSL7, MEDI7814, aurin tricarboxylic acid, an aurin tricarboxylic acid derivative, RG6107/SKY59, ALXN1210, ALXN5500, TT30, ABP959, Anti-05 siRNA, Erdigna, avacincaptad pegol/Zimura®, SOBI005, ISU305, and REGN3918.

65. The method of any one of claims 60-64, wherein the fD inhibitor is selected from BioCryst Pharmaceuticals fD inhibitor, a Novartis fD inhibitor, a Bristol-Myers Squibb fD inhibitor, a Japan Tobacco Inc. fD inhibitor, FCFD4515S, nafomostat, SOMAmers for fD (SomaLogic), lampalizumab, aptamers to fD (Vitrisia Therapeutics), a Ra Pharmaceutical fD inhibitor, an Alexion Pharmaceuticals fD inhibitor, and an Achillion Pharmaceuticals fD inhibitor.

66. The method of any one of claims 60-64, wherein the fD inhibitor is a compound of Formula I: 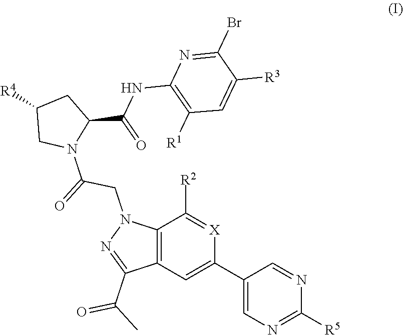
or a pharmaceutically acceptable salt, N-oxide, isotopic derivative, or prodrug thereof;
wherein:
X is selected from N and CH;
R¹ is selected from hydrogen, C1-C3 alkyl, and halogen;
R² is selected from hydrogen and C1-C3 alkyl;
R³ is selected from hydrogen, C1-C3 alkyl, and halogen;
R⁴ is selected from hydrogen, C1-C3 alkyl, and halogen; and
R⁵ is selected from hydrogen, C1-C3 alkyl, halogen, and cyano.

67. The method of claim 66, wherein the compound is selected from:

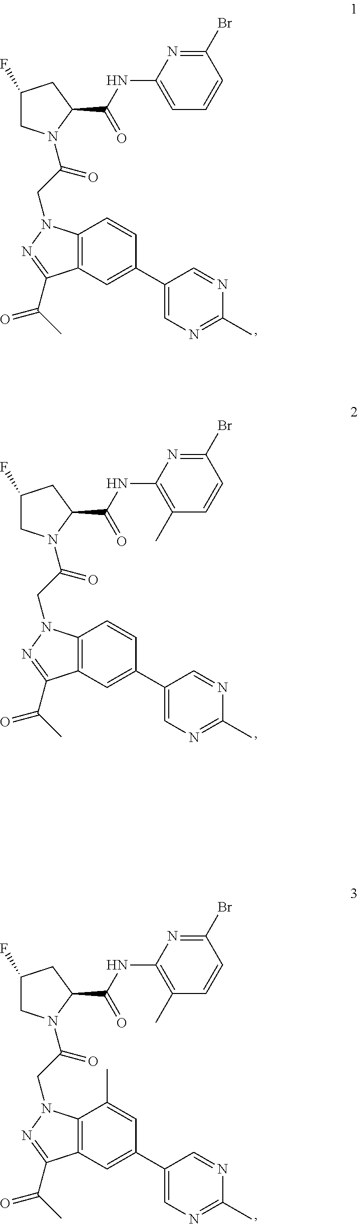 [SMILES: CC(=O)c1nn(CC(=O)N2C[C@H](F)C[C@H]2C(=O)Nc2cccc(Br)n2)c2ccc(-c3cnc(C)nc3)cc12 ; CC(=O)c1nn(CC(=O)N2C[C@H](F)C[C@H]2C(=O)Nc2nc(Br)ccc2C)c2ccc(-c3cnc(C)nc3)cc12 ; CC(=O)c1nn(CC(=O)N2C[C@H](F)C[C@H]2C(=O)Nc2nc(Br)ccc2C)c2c(C)cc(-c3cnc(C)nc3)cc12]

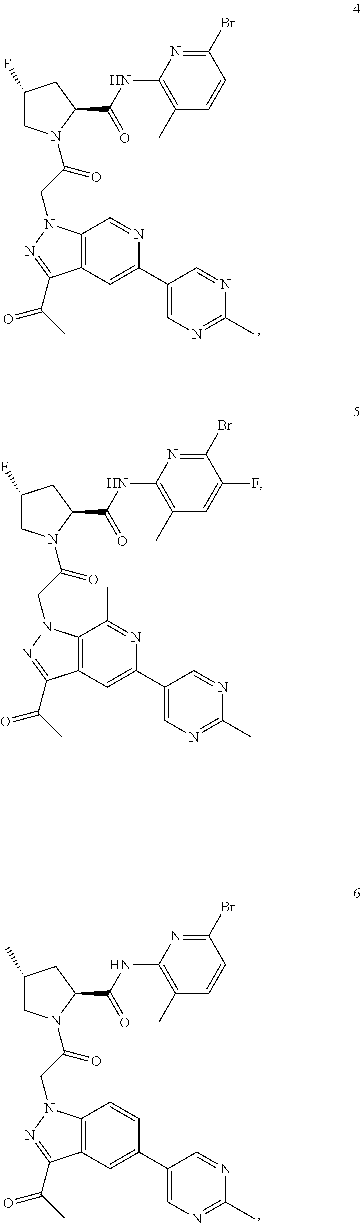

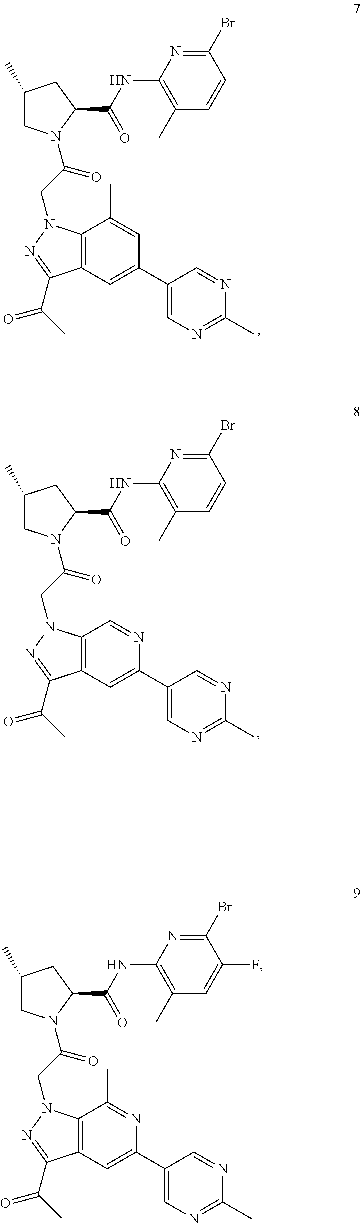

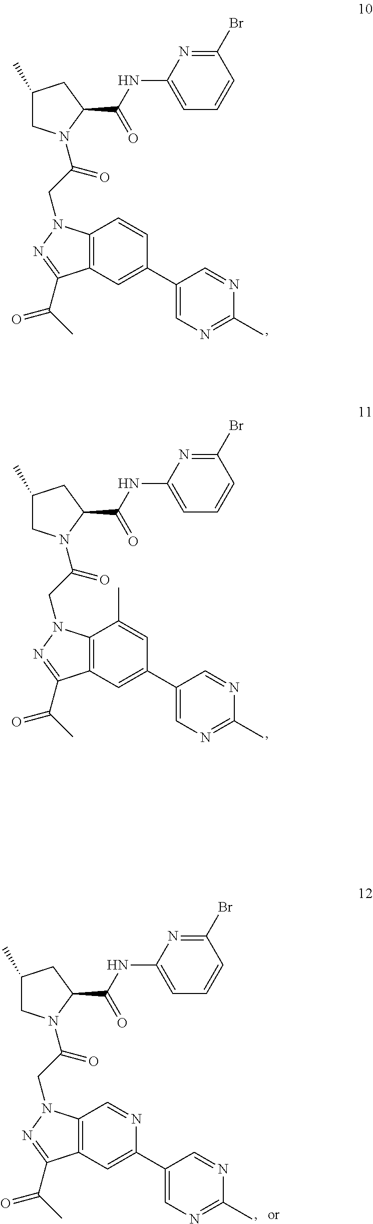

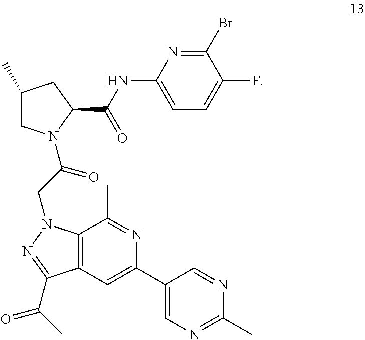 [SMILES: CC(=O)c1nn(CC(=O)N2C[C@H](C)C[C@H]2C(=O)Nc2ccc(F)c(Br)n2)c2c(C)nc(-c3cnc(C)nc3)cc12]

68. The method of any one of claims 60-64, wherein the fD inhibitor is a compound of Formula II: 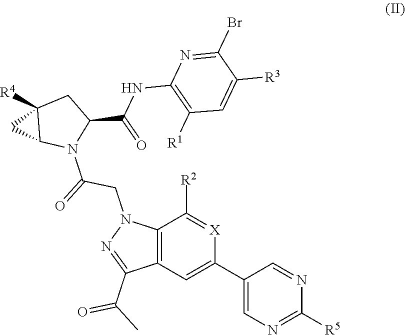
or a pharmaceutically acceptable salt, N-oxide, isotopic derivative, or prodrug thereof;
wherein:
X is selected from N and CH;
R¹ is selected from hydrogen, C₁-C₃ alkyl, and halogen;
R² is selected from hydrogen and C₁-C₃ alkyl;
R³ is selected from hydrogen, C₁-C₃ alkyl, and halogen;
R⁴ is selected from hydrogen, C₁-C₃ alkyl, and halogen; and
R⁵ is selected from hydrogen, C₁-C₃ alkyl, halogen, and cyano.

69. The method of claim 68, wherein the compound is selected from:

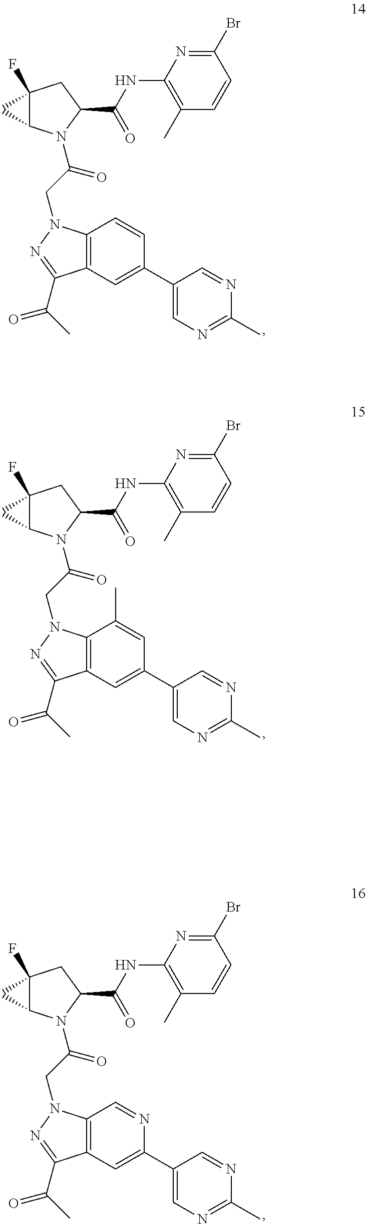 [SMILES: CC(=O)c1nn(CC(=O)N2[C@H](C(=O)Nc3nc(Br)ccc3C)C[C@@]3(F)C[C@@H]23)c2ccc(-c3cnc(C)nc3)cc12 ; CC(=O)c1nn(CC(=O)N2[C@H](C(=O)Nc3nc(Br)ccc3C)C[C@@]3(F)C[C@@H]23)c2c(C)cc(-c3cnc(C)nc3)cc12 ; CC(=O)c1nn(CC(=O)N2[C@H](C(=O)Nc3nc(Br)ccc3C)C[C@@]3(F)C[C@@H]23)c2cnc(-c3cnc(C)nc3)cc12]

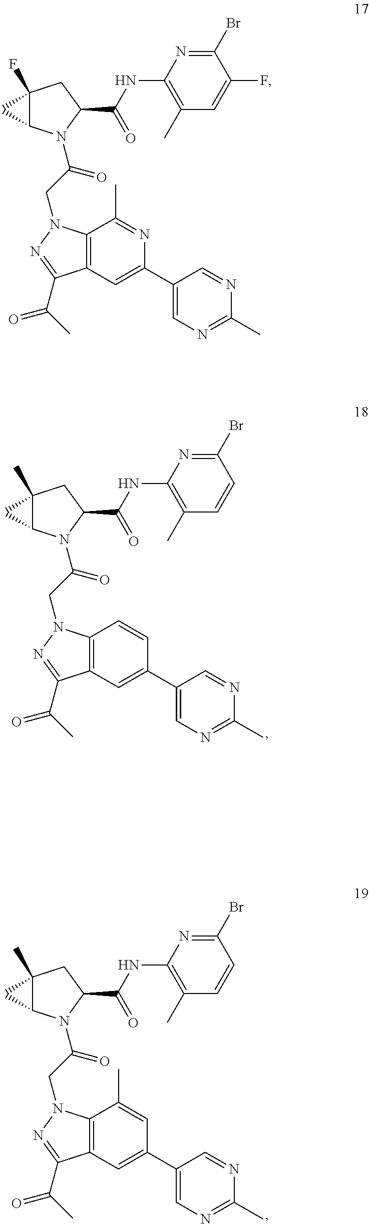 [SMILES: CC(=O)c1nn(CC(=O)N2[C@H](C(=O)Nc3nc(Br)c(F)cc3C)C[C@@]3(F)C[C@@H]23)c2c(C)nc(-c3cnc(C)nc3)cc12 ; CC(=O)c1nn(CC(=O)N2[C@H](C(=O)Nc3nc(Br)ccc3C)C[C@@]3(C)C[C@@H]23)c2ccc(-c3cnc(C)nc3)cc12 ; CC(=O)c1nn(CC(=O)N2[C@H](C(=O)Nc3nc(Br)ccc3C)C[C@@]3(C)C[C@@H]23)c2c(C)cc(-c3cnc(C)nc3)cc12]

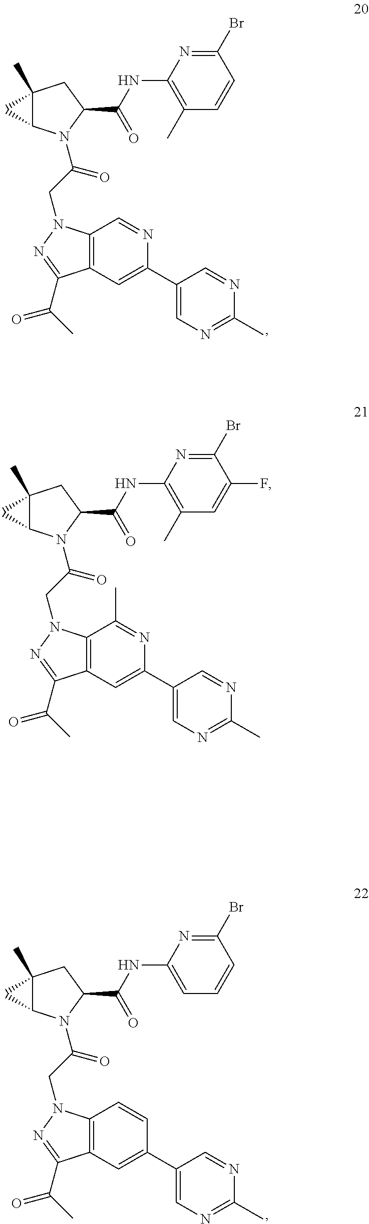 [SMILES: CC(=O)c1nn(CC(=O)N2[C@H](C(=O)Nc3nc(Br)ccc3C)C[C@@]3(C)C[C@@H]23)c2cnc(-c3cnc(C)nc3)cc12 ; CC(=O)c1nn(CC(=O)N2[C@H](C(=O)Nc3nc(Br)c(F)cc3C)C[C@@]3(C)C[C@@H]23)c2c(C)nc(-c3cnc(C)nc3)cc12 ; CC(=O)c1nn(CC(=O)N2[C@H](C(=O)Nc3cccc(Br)n3)C[C@@]3(C)C[C@@H]23)c2ccc(-c3cnc(C)nc3)cc12]

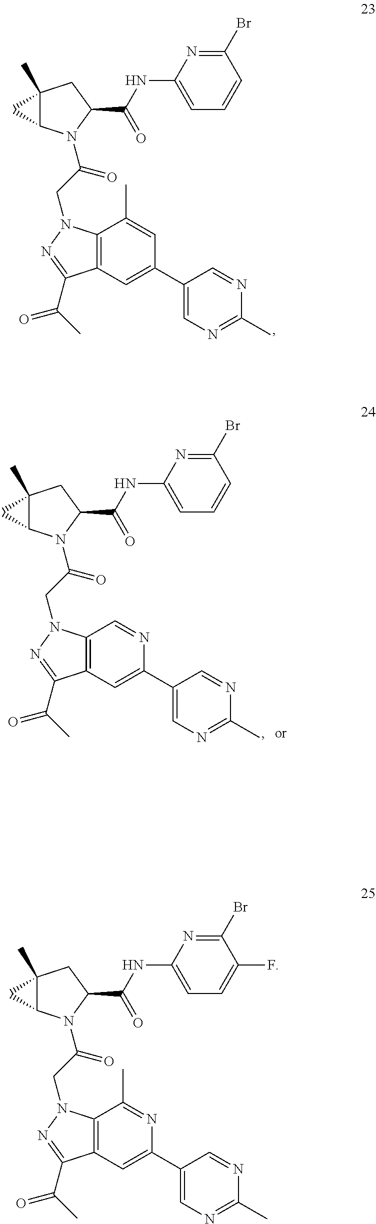 [SMILES: CC(=O)c1nn(CC(=O)N2[C@H](C(=O)Nc3cccc(Br)n3)C[C@@]3(C)C[C@@H]23)c2c(C)cc(-c3cnc(C)nc3)cc12 ; CC(=O)c1nn(CC(=O)N2[C@H](C(=O)Nc3cccc(Br)n3)C[C@@]3(C)C[C@@H]23)c2cnc(-c3cnc(C)nc3)cc12 ; CC(=O)c1nn(CC(=O)N2[C@H](C(=O)Nc3ccc(F)c(Br)n3)C[C@@]3(C)C[C@@H]23)c2c(C)nc(-c3cnc(C)nc3)cc12]

70. The method of any one of claims 60-69, wherein the AP-associated nephropathy is C3 glomerulonephritis (C3GN).

71. The method of any one of claims 60-69, wherein the AP-associated nephropathy is dense deposit disease (DDD).

72. The method of any one of claims 60-69, wherein the AP-associated nephropathy is immune complex membranoproliferative glomerulonephritis (IC-MPGN).

73. The method of any one of claims 60-69, wherein the AP-associated nephropathy is selected from: atypical or typical hemolytic uremic syndrome (HUS); lupus nephritis resulting from systemic lupus erythematous (SLE); IgA nephropathy; anti-neutrophilic cytoplasmic autoantibody (ANCA) glomerulonephritis; scleroderma renal crisis; post-infectious glomerulonephritis; glomerulonephritis and vasculitis resulting from Henoch-Schonlein purpura (HSP); anti-glomerular basement membrane (GBM) or Goodpasture's disease; light chain deposition disease; contrast-induced nephropathy (CIN); membranous glomerulonephritis; cryoglobulinemia; pre-eclampsia and eclampsia; and minimal change disease.

74. The method of any one of claims 60-69, wherein the AP-associated nephropathy is a disorder of kidney transplantation selected from delayed graft function (DGF), antibody-mediated rejection (AMR), or graft-versus-host disease (GvHD).

75. A method for the diagnosis of a human subject suffering from a suspected alternative pathway (AP)-associated nephropathy comprising:
i. analyzing the levels in the urine of the subject of at least two biomarkers selected from Ba, sC5b-9, and C3c;
ii. comparing the subject's at least two biomarker levels to a range of the at least two biomarker levels derived from individuals that do not have an AP-associated nephropathy (“normal range”); and
iii. if the subject's at least two biomarker levels are greater than the normal range, diagnosing the subject with the AP-associated nephropathy.

76. The method of claim 75, wherein the AP-associated nephropathy is C3 glomerulonephritis (C3GN).

77. The method of claim 75, wherein the AP-associated nephropathy is dense deposit disease (DDD).

78. The method of claim 75, wherein the AP-associated nephropathy is immune complex membranoproliferative glomerulonephritis (IC-MPGN).

79. The method of claim 75, wherein the AP-associated nephropathy is selected from: atypical or typical hemolytic uremic syndrome (HUS); lupus nephritis resulting from systemic lupus erythematous (SLE); IgA nephropathy; anti-neutrophilic cytoplasmic autoantibody (ANCA) glomerulonephritis; scleroderma renal crisis; post-infectious glomerulonephritis;
glomerulonephritis and vasculitis resulting from Henoch-Schonlein purpura (HSP); anti-glomerular basement membrane (GBM) or Goodpasture's disease; light chain deposition disease; contrast-induced nephropathy (CIN); membranous glomerulonephritis;
cryoglobulinemia; pre-eclampsia and eclampsia; and minimal change disease.

80. The method of claim 75, wherein the AP-associated nephropathy is a disorder of kidney transplantation selected from delayed graft function (DGF), antibody-mediated rejection (AMR), or graft-versus-host disease (GvHD).

81. A method for the targeted selection and treatment of a human subject suffering from a suspected alternative pathway (AP)-associated nephropathy comprising:
i. analyzing the levels in the urine of the subject of at least two biomarkers selected from Ba, sC5b-9, C3c;
ii. comparing the subject's at least two biomarker levels to a range of the same biomarker levels derived from individuals that do not have an AP-associated nephropathy (“normal range”); and
iii. if the subject's at least two biomarker levels are greater than the normal range, administering to the subject an AP inhibitor.

82. The method of claim 81, wherein the subject's urine biomarker levels are at least 2×, 3×, 4×, 5×, 6×, 7×, 8×, 9×, or 10× higher than the upper limit of the normal range.

83. The method of claim 81, wherein the subject's urine biomarker levels are at least 100×, 200×, 300×, 400×, or 500× higher than the upper limit of the normal range.

84. A method for the targeted selection and treatment of a human subject suffering from a suspected alternative pathway (AP)-associated nephropathy comprising:
i. analyzing the levels in the urine of the subject of at least two biomarkers selected from Ba, sC5b-9, C3c;
ii. comparing the subject's at least two biomarker levels to a range of the at least two biomarker levels derived from individuals that have an AP-associated nephropathy (“abnormal range”); and
iii. if the subject's at least two biomarker levels fall within the abnormal range, administering to the subject an AP inhibitor.

85. A method of monitoring the efficacy of an alternative pathway (AP) inhibitor treatment regimen in a human subject having an AP-associated nephropathy and receiving an AP inhibitor comprising:
i. analyzing the levels in a first urine sample from the subject of at least two biomarkers selected from Ba, sC5b-9, C3c;
ii. analyzing levels of the at least two biomarkers in a subsequent urine sample from the subject, wherein the first urine sample is collected prior to the collection of the subsequent urine sample;
iii. comparing the at least two biomarker levels in the first urine sample to the at least two biomarker levels in the subsequent urine sample; and
iv. if the at least two biomarker levels in the subsequent urine sample are equal to or greater than the at least two biomarker levels in the first urine sample, then increasing the dose of the AP inhibitor being administered to the subject.

86. A method of monitoring the efficacy of an alternative pathway (AP) inhibitor treatment regimen in a human subject having an AP-associated nephropathy and receiving an AP inhibitor comprising:
i. analyzing the levels in a first urine sample from the subject of at least two biomarkers selected from Ba, sC5b-9, C3c;
iii. analyzing levels of the at least two biomarkers in a subsequent urine sample from the subject, wherein the first urine sample is collected prior to the collection of the subsequent urine sample;
ii. comparing the at least two biomarker levels in the first urine sample to the at least two biomarker levels in the subsequent urine sample; and
iv. if the at least two biomarker levels in the subsequent urine sample have insufficiently decreased compared to the at least two biomarker levels in the first urine sample, then increasing the dose of the AP inhibitor being administered to the subject.

87. The method of claim 86, wherein at least one of the biomarker levels in the subsequent urine sample have decreased by less than 10%, 20%, 30%, 40%, 50%, 60%, 70%, 80%, or 90% of the biomarker levels in the first urine sample.

88. A method for the prediction of a long-term benefit of a human subject suffering from an alternative pathway (AP)-associated nephropathy comprising:
i. treating the subject suffering from the AP-associated nephropathy with an AP inhibitor regimen;
ii. analyzing the levels in the urine of the subject of a at least two biomarkers selected from Ba, sC5b-9, and C3c, or a combination thereof; and
iii. maintaining the subject's at least two biomarker levels within a range of the at least two biomarker levels derived from individuals that do not have an AP-associated nephropathy (“normal range”).

89. The method of any one of claims 81-88, wherein the biomarkers are Ba and sC5b-9.

90. The method of any one of claims 81-88, wherein the biomarkers are Ba and C3c.

91. The method of any one of claims 81-88, wherein the biomarkers are sC5b-9 and C3c.

92. The method of any one of claims 81-88, wherein the biomarkers are Ba, sC5b-9, and C3c.

93. The method of any one of claims 81-92, wherein the biomarker levels are normalized.

94. The method of claim 93, wherein the biomarker levels are normalized to urine creatinine.

95. The method of claim 93, wherein the biomarker levels are normalized to urine albumin.

96. The method of claim 93, wherein the biomarker levels are normalized to both urine albumin and urine creatinine.

97. The method of any one of claims 81-96, wherein the AP inhibitor is a factor D (fD) inhibitor.

98. The method of claim 97, wherein the fD inhibitor is selected from BioCryst Pharmaceuticals fD inhibitor, a Novartis fD inhibitor, a Bristol-Myers Squibb fD inhibitor, a Japan Tobacco Inc. fD inhibitor, FCFD4515S, nafomostat, SOMAmers for fD (SomaLogic), lampalizumab, aptamers to fD (Vitrisia Therapeutics), a Ra Pharmaceutical fD inhibitor, an Alexion Pharmaceuticals fD inhibitor, and an Achillion Pharmaceuticals fD inhibitor.

99. The method of claim 97, wherein the fD inhibitor is a compound of Formula I: 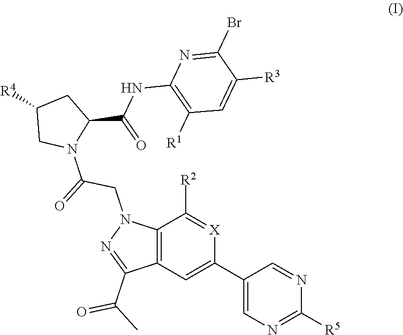
or a pharmaceutically acceptable salt, N-oxide, isotopic derivative, or prodrug thereof;
wherein:
X is selected from N and CH;
R¹ is selected from hydrogen, C₁-C₃ alkyl, and halogen;
R² is selected from hydrogen and C₁-C₃ alkyl;
R³ is selected from hydrogen, C₁-C₃ alkyl, and halogen;
R⁴ is selected from hydrogen, C₁-C₃ alkyl, and halogen; and
R⁵ is selected from hydrogen, C₁-C₃ alkyl, halogen, and cyano.

100. The method of claim 99, wherein the compound is selected from:

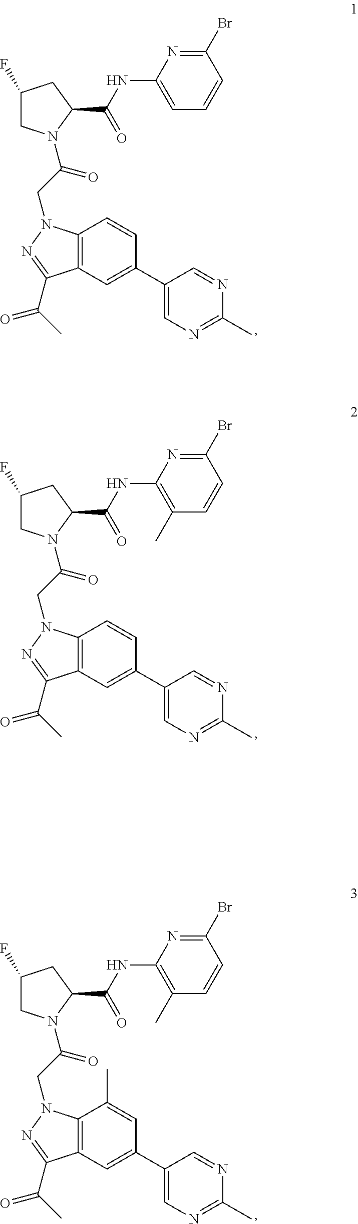 [SMILES: CC(=O)c1nn(CC(=O)N2C[C@H](F)C[C@H]2C(=O)Nc2cccc(Br)n2)c2ccc(-c3cnc(C)nc3)cc12 ; CC(=O)c1nn(CC(=O)N2C[C@H](F)C[C@H]2C(=O)Nc2nc(Br)ccc2C)c2ccc(-c3cnc(C)nc3)cc12 ; CC(=O)c1nn(CC(=O)N2C[C@H](F)C[C@H]2C(=O)Nc2nc(Br)ccc2C)c2c(C)cc(-c3cnc(C)nc3)cc12]

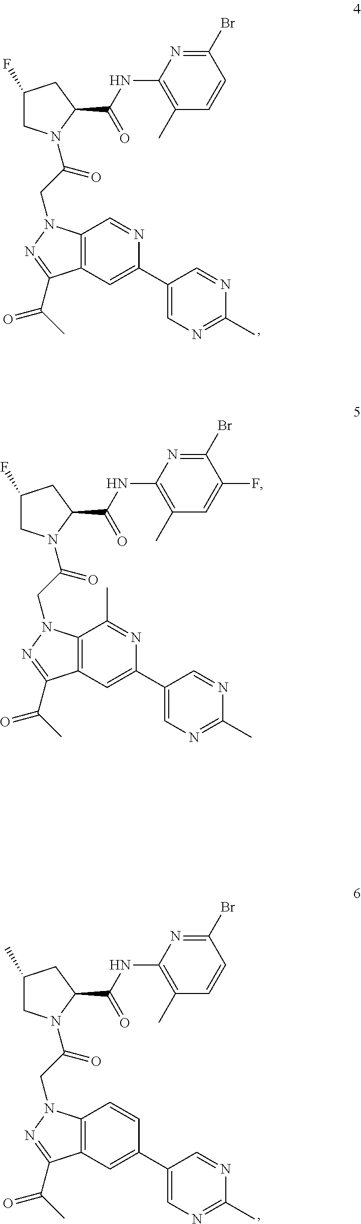

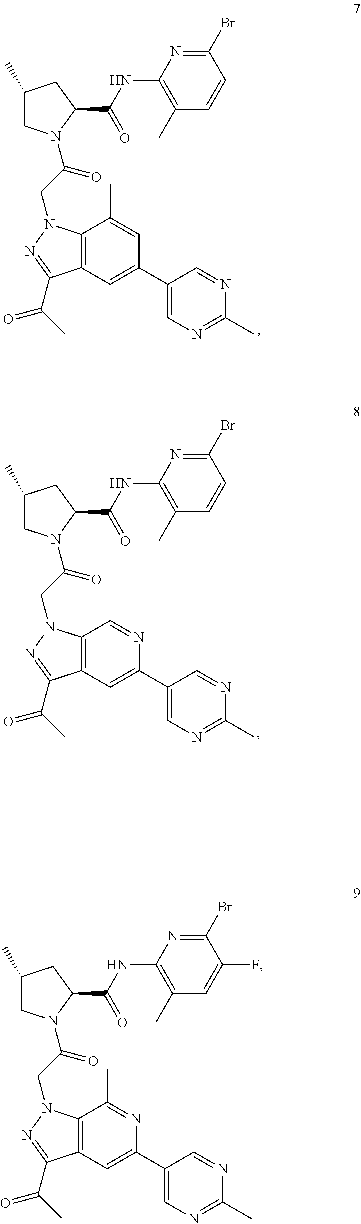

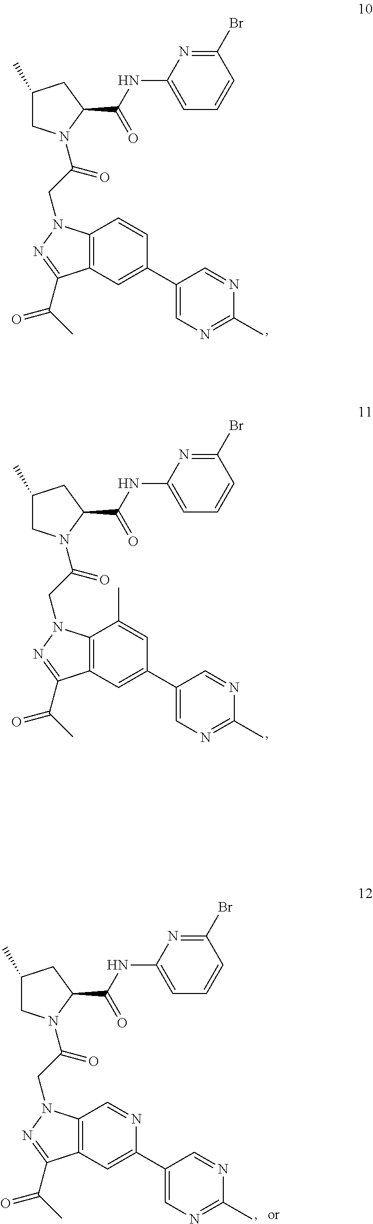

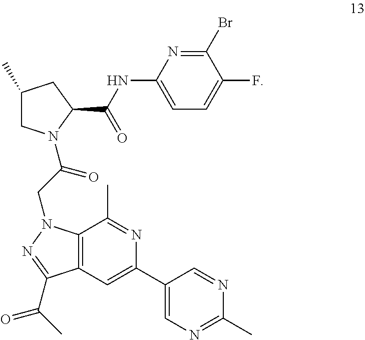 [SMILES: CC(=O)c1nn(CC(=O)N2C[C@H](C)C[C@H]2C(=O)Nc2ccc(F)c(Br)n2)c2c(C)nc(-c3cnc(C)nc3)cc12]

101. The method of claim 99, wherein the fD inhibitor is a compound of Formula II: 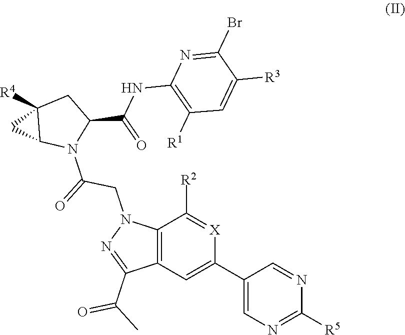
or a pharmaceutically acceptable salt, N-oxide, isotopic derivative, or prodrug thereof;
wherein:
X is selected from N and CH;
R¹ is selected from hydrogen, C₁-C₃ alkyl, and halogen;
R² is selected from hydrogen and C₁-C₃ alkyl;
R³ is selected from hydrogen, C₁-C₃ alkyl, and halogen;
R⁴ is selected from hydrogen, C₁-C₃ alkyl, and halogen; and
R⁵ is selected from hydrogen, C₁-C₃ alkyl, halogen, and cyano.

102. The method of claim 101, wherein the compound is selected from:

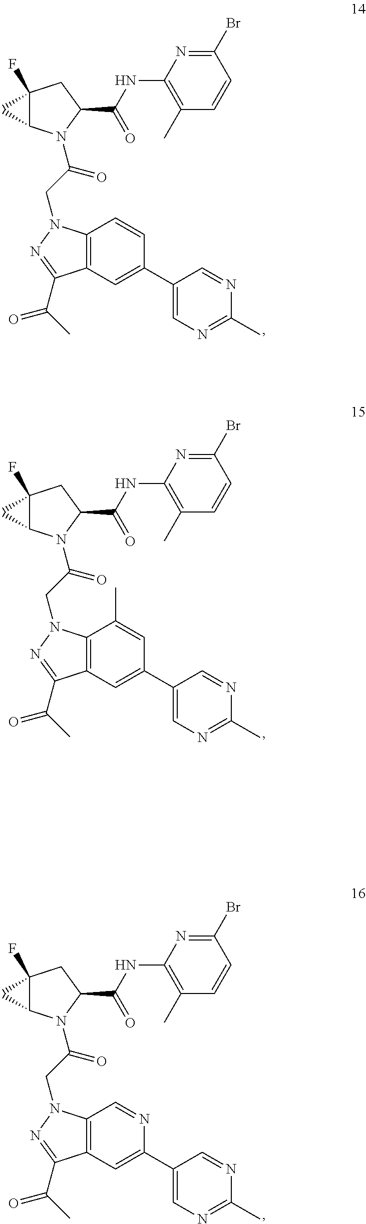 [SMILES: CC(=O)c1nn(CC(=O)N2[C@H](C(=O)Nc3nc(Br)ccc3C)C[C@@]3(F)C[C@@H]23)c2ccc(-c3cnc(C)nc3)cc12 ; CC(=O)c1nn(CC(=O)N2[C@H](C(=O)Nc3nc(Br)ccc3C)C[C@@]3(F)C[C@@H]23)c2c(C)cc(-c3cnc(C)nc3)cc12 ; CC(=O)c1nn(CC(=O)N2[C@H](C(=O)Nc3nc(Br)ccc3C)C[C@@]3(F)C[C@@H]23)c2cnc(-c3cnc(C)nc3)cc12]

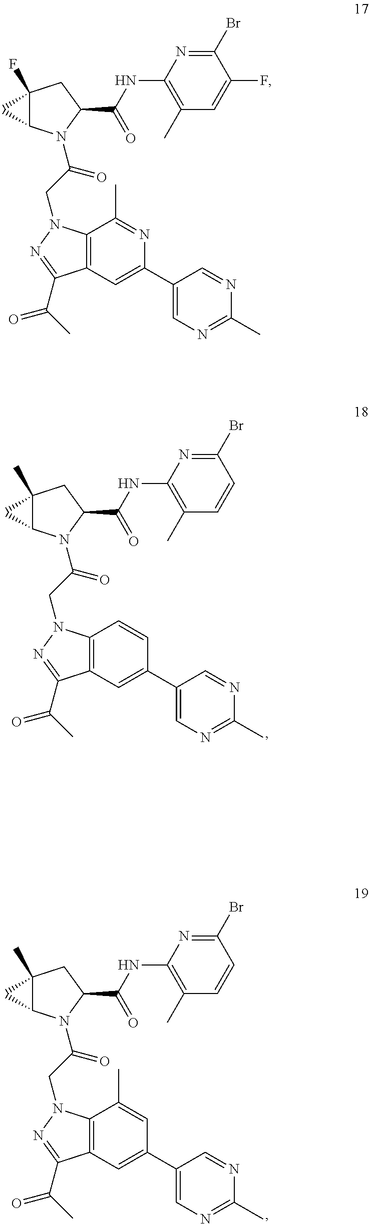 [SMILES: CC(=O)c1nn(CC(=O)N2[C@H](C(=O)Nc3nc(Br)c(F)cc3C)C[C@@]3(F)C[C@@H]23)c2c(C)nc(-c3cnc(C)nc3)cc12 ; CC(=O)c1nn(CC(=O)N2[C@H](C(=O)Nc3nc(Br)ccc3C)C[C@@]3(C)C[C@@H]23)c2ccc(-c3cnc(C)nc3)cc12 ; CC(=O)c1nn(CC(=O)N2[C@H](C(=O)Nc3nc(Br)ccc3C)C[C@@]3(C)C[C@@H]23)c2c(C)cc(-c3cnc(C)nc3)cc12]

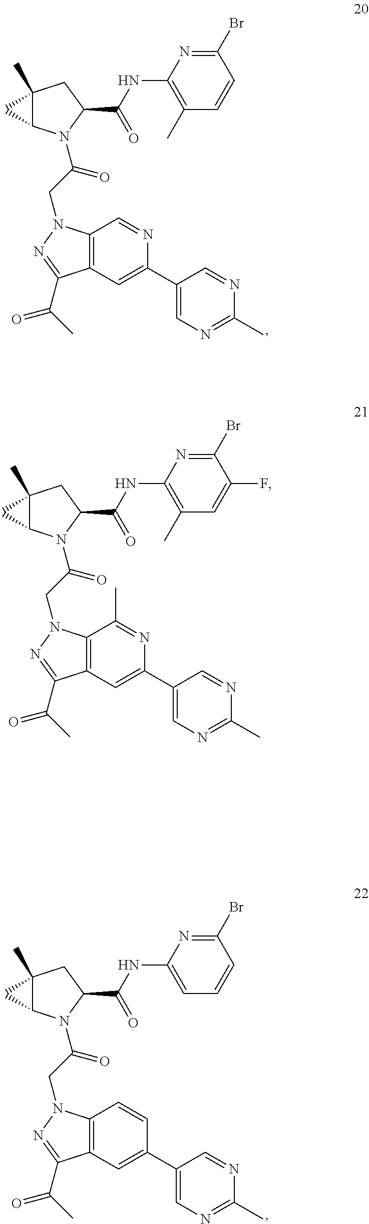 [SMILES: CC(=O)c1nn(CC(=O)N2[C@H](C(=O)Nc3nc(Br)ccc3C)C[C@@]3(C)C[C@@H]23)c2cnc(-c3cnc(C)nc3)cc12 ; CC(=O)c1nn(CC(=O)N2[C@H](C(=O)Nc3nc(Br)c(F)cc3C)C[C@@]3(C)C[C@@H]23)c2c(C)nc(-c3cnc(C)nc3)cc12 ; CC(=O)c1nn(CC(=O)N2[C@H](C(=O)Nc3cccc(Br)n3)C[C@@]3(C)C[C@@H]23)c2ccc(-c3cnc(C)nc3)cc12]

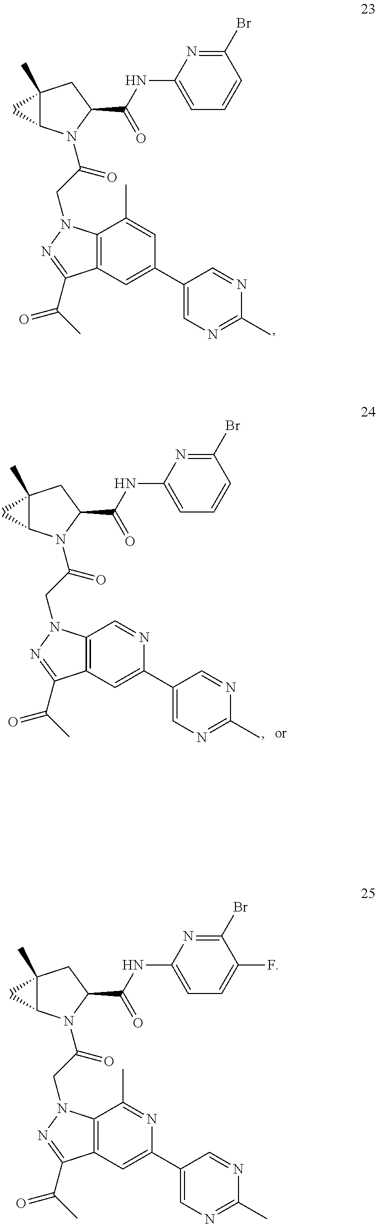 [SMILES: CC(=O)c1nn(CC(=O)N2[C@H](C(=O)Nc3cccc(Br)n3)C[C@@]3(C)C[C@@H]23)c2c(C)cc(-c3cnc(C)nc3)cc12 ; CC(=O)c1nn(CC(=O)N2[C@H](C(=O)Nc3cccc(Br)n3)C[C@@]3(C)C[C@@H]23)c2cnc(-c3cnc(C)nc3)cc12 ; CC(=O)c1nn(CC(=O)N2[C@H](C(=O)Nc3ccc(F)c(Br)n3)C[C@@]3(C)C[C@@H]23)c2c(C)nc(-c3cnc(C)nc3)cc12]

103. The method of any one of claims 81-96, wherein the AP inhibitor is a factor B (fB) inhibitor.

104. The method of claim 103, wherein the fB inhibitor is selected from anti-FB siRNA, TA106, LNP106, LNP023, complin, and Ionis-FB-L_{Rx}.

105. The method of claim 103, wherein the fB inhibitor is a compound of the formula:

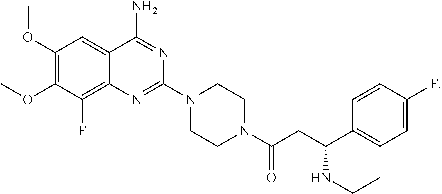 [SMILES: CCN[C@H](CC(=O)N1CCN(c2nc(N)c3cc(OC)c(OC)c(F)c3n2)CC1)c1ccc(F)cc1]

106. The method of any one of claims 81-96, wherein the AP inhibitor is a complement component 3 (C3) inhibitor.

107. The method of claim 106, wherein the C3 inhibitor is selected from compstatin, 4(1MeW)/APL-1, Cp40/AMY-101, PEG-Cp40, 4(1MeW) POT-4, and AMY-201.

108. The method of claim 106, wherein the C3 inhibitor is selected from selected from H17, mirocept, sCR1, TT32, HC-1496, CB-2782, and APL-2.

109. The method of any one of claims 81-96, wherein the AP inhibitor is a C3 convertase inhibitor.

110. The method of claim 109, wherein the C3 convertase inhibitor is selected from CRIg/CFH, Mini-CFH, TT30, and rFH (Optherion).

111. The method of any one of claims 81-96, wherein the AP inhibitor is a complement component 3b (C3b) inhibitor.

112. The method of claim 111, wherein the C3b inhibitor is selected from APL-2, 4(1MeW) POT-4, PEG-Cp40, H17, ALXN1102/ALXN1103, and rFH.

113. The method of any one of claims 81-112, wherein the AP-associated nephropathy is C3 glomerulonephritis (C3GN).

114. The method of any one of claims 81-112, wherein the AP-associated nephropathy is dense deposit disease (DDD).

115. The method of any one of claims 81-112, wherein the AP-associated nephropathy is immune complex membranoproliferative glomerulonephritis (IC-MPGN).

116. The method of any one of claims 81-112, wherein the AP-associated nephropathy is selected from: atypical or typical hemolytic uremic syndrome (HUS); lupus nephritis resulting from systemic lupus erythematous (SLE); IgA nephropathy; anti-neutrophilic cytoplasmic autoantibody (ANCA) glomerulonephritis; scleroderma renal crisis; post-infectious glomerulonephritis; glomerulonephritis and vasculitis resulting from Henoch-Schonlein purpura (HSP); anti-glomerular basement membrane (GBM) or Goodpasture's disease; light chain deposition disease; contrast-induced nephropathy (CIN); membranous glomerulonephritis; cryoglobulinemia; pre-eclampsia and eclampsia; and minimal change disease.

117. The method of any one of claims 81-112, wherein the AP-associated nephropathy is a disorder of kidney transplantation selected from delayed graft function (DGF), antibody-mediated rejection (AMR), or graft-versus-host disease (GvHD).

118. A method for the targeted selection and treatment of a human subject suffering from a suspected alternative pathway (AP)-associated nephropathy comprising:
i. analyzing the levels in the urine of the subject of C3c;
ii. comparing the subject's C3c levels to a range of C3c levels derived from individuals that do not have an AP-associated nephropathy (“normal range”); and
iii. if the subject's C3c levels are greater than the normal range, administering to the subject an AP inhibitor.

119. A method for diagnosing a human subject suspected of having an alternative pathway (AP)-associated nephropathy comprising:
i. analyzing the levels in the urine of the subject of C3c;
ii. comparing the subject's C3c levels to a range of C3c levels derived from individuals that do not have an AP-associated nephropathy (“normal range”); and
iii. if the subject's C3c levels are greater than the normal range, diagnosing the subject with an AP-associated nephropathy.
